(19) United States
(12) Patent Application Publication
(10) Pub. No.: US 2016/0024157 A1
(43) Pub. Date: Jan. 28, 2016

(54) HAEMOPHILUS INFLUENZAE TYPE B

(71) Applicants: GLAXOSMITHKLINE BIOLOGICALS SA, Rixensart (BE); J. CRAIG VENTER INSTITUTE, INC., Rockville, MD (US)

(72) Inventors: Vega MASIGNANI, Siena (IT); Rino RAPPUOLI, Siena (IT); Herve TETTELIN, Gaithersburg, MD (US)

(73) Assignees: GLAXOSMITHKLINE BIOLOGICALS SA, Rixensart (BE); J. CRAIG VENTER INSTITUTE, INC., Rockville, MD (US)

(21) Appl. No.: 14/876,623

(22) Filed: Oct. 6, 2015

Related U.S. Application Data
(63) Continuation of application No. 14/309,829, filed on Jun. 19, 2014, now abandoned.
Application No. 14/309,829 is a division of application No. 13/333,815, filed on Dec. 21, 2011, now abandoned.
Application No. 13/333,815 is a division of application No. 11/887,712, filed on May 19, 2009, now abandoned.
(60) Provisional application No. 60/667,921, filed on Mar. 30, 2005.

(51) Int. Cl. C07K 14/285; A61K 39/102; C07K 16/12

(52) U.S. Cl. CPC C07K 14/285; C07K 16/1242; A61K 39/102

(57) ABSTRACT

Polypeptides comprising various amino acid sequences derived from Haemophilus influenzae type b, including a number of lipoproteins. These can be used in the development of vaccines for preventing and/or treating bacterial meningitis. They may also be useful for diagnostic purposes, and as targets for antibiotics. Antibodies against the polypeptides are also disclosed, as are the coding nucleic acids.

### CROSS REFERENCE TO RELATED APPLICATIONS

This application is a Continuation of U.S. patent application Ser. No. 14/309,829, filed Jun. 19, 2014; which is a Divisional of U.S. patent application Ser. No. 13/333,815, filed Dec. 21, 2011, now abandoned; which is a Divisional of U.S. patent application Ser. No. 11/887,712, claiming an international filing date of Mar. 30, 2006, now abandoned; which is the National Stage of International Patent Application PCT/US2006/012606, filed Mar. 30, 2006; which claims priority to U.S. Provisional Patent Application Ser. No. 60/667,921, filed Mar. 30, 2005; all of which are hereby incorporated by reference in their entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 529552001401SeqList.TXT, date recorded: Oct. 5, 2015, size: 9,133 KB).

### TECHNICAL FIELD

This invention is in the field of Haemophilus influenzae immunology and vaccinology.

### BACKGROUND ART

Haemophilus influenzae is a small, non-motile, Gram-negative coccobacillus. It is a respiratory pathogen that causes a wide spectrum of human infections, including: asymptomatic colonization of the upper respiratory tract (i.e. carriage); infections that extend from colonized mucosal surfaces to cause otitis media (inflammation of the middle ear), bronchitis, conjunctivitis, sinusitis, urinary tract infections and pneumonia; and invasive infections, such as bacteremia, septic arthritis, epiglottitis, pneumonia, empyema, pericarditis, cellulitis, osteomyelitis and meningitis. H. influenzae was the first bacterium for which a complete genome sequence was published [1].

H. influenzae strains are either capsulated (typeable) or non-capsulated (non-typeable), and there are six major serological types of capsulated strains (a to f). 95% of H. influenzae-caused invasive diseases are caused by H. influenzae type B (‘Hib’) strains. The most serious manifestation of Hib disease is meningitis, but the introduction in the 1980s of vaccines based on conjugated Hib capsular saccharides has hugely reduced incidence of this disease. Manufacture of the conjugated vaccine involves separate preparation of saccharide and carrier, followed by conjugation, and a simple protein antigen would be more convenient in manufacturing terms.

The genome sequence of the serotype d strain KW20 [1,2] has been useful for understanding basic H. influenzae biology, but it has not been so useful in countering pathogenic H. influenzae strains, as serotype d strains are generally not pathogens.

It is an object of the invention to provide polypeptides for use in the development of vaccines for preventing and/or treating infections caused by type b H. influenzae strains. In particular, it is an object to provide polypeptides for use in improved vaccines for preventing and/or treating bacterial meningitis caused by Hib. The polypeptides may also be useful for diagnostic purposes, and as targets for antibiotics.

### DISCLOSURE OF THE INVENTION

### Polypeptides

The invention provides polypeptides comprising the H. influenzae amino acid sequences disclosed in the examples. These amino acid sequences are the even SEQ ID NOs between 2 and 3706. There are thus 1853 amino acid sequences, and these are referred to as HlBnnnn, where nnnn is a number between 0001 and 1853.

The invention also provides polypeptides comprising amino acid sequences that have sequence identity to the H. influenzae amino acid sequences disclosed in the examples. Depending on the particular sequence, the degree of sequence identity is preferably greater than 50% (e.g. 60%, 70%, 75%, 80%, 85%, 90%, 91%, 92%, 93%©, 94%, 95%, 96%, 97%, 98%, 99% or more). These polypeptides include homologs, orthologs, allelic variants and functional mutants. Typically, 50% identity or more between two polypeptide sequences is considered to be an indication of functional equivalence. Identity between polypeptides is preferably determined by the Smith-Waterman homology search algorithm as implemented in the MPSRCH program (Oxford Molecular), using an affine gap search with parameters gap open penalty=12 and gap extension penalty=1.

These polypeptide may, compared to the Hib sequences of the examples, include one or more (e.g. 1, 2, 3, 4, 5, 6, 7, 8, 9, 10, etc.) conservative amino acid replacements i.e. replacements of one amino acid with another which has a related side chain. Genetically-encoded amino acids are generally divided into four families: (1) acidic i.e. aspartate, glutamate; (2) basic i.e. lysine, arginine, histidine; (3) non-polar i.e. alanine, valine, leucine, isoleucine, proline, phenylalanine, methionine, tryptophan; and (4) uncharged polar i.e. glycine, asparagine, glutamine, cysteine, serine, threonine, tyrosine. Phenylalanine, tryptophan, and tyrosine are sometimes classified jointly as aromatic amino acids. In general, substitution of single amino acids within these families does not have a major effect on the biological activity. The polypeptides may have one or more (e.g. 1, 2, 3, 4, 5, 6, 7, 8, 9, 10, etc.) single amino acid deletions relative to the Hib sequences of the examples. The polypeptides may also include one or more (e.g. 1, 2, 3, 4, 5, 6, 7, 8, 9, 10, etc.) insertions (e.g. each of 1, 2, 3, 4 or 5 amino acids) relative to the Hib sequences of the examples.

Preferred polypeptides of the invention are listed below, including polypeptides that are lipidated, that are located in the outer membrane, that are located in the inner membrane, or that are located in the periplasm. Particularly preferred polypeptides are those that fall into more than one of these categories e.g. lipidated polypeptides that are located in the outer membrane, such as HIB0374, HIB0382, HIB0426, HIB0733, HIB0734, HIB1564 and HIB1654. Two preferred lipoproteins are HIB1027 and HIB1255. Lipoproteins may have a N-terminal cysteine to which lipid is covalenty attached, following post-translational processing of the signal peptide.

The invention further provides polypeptides comprising fragments of the H. influenzae amino acid sequences disclosed in the examples. The fragments should comprise at least n consecutive amino acids from the sequences and, depending on the particular sequence, n is 7 or more (e.g. 8, 10, 12, 14, 16, 18, 20, 22, 24, 26, 28, 30, 35, 40, 45, 50, 60, 70, 80, 90, 100 or more).

The fragment may comprise at least one T-cell or, preferably, a B-cell epitope of the sequence. T- and B-cell epitopes can be identified empirically (e.g. using PEPSCAN [3,4] or similar methods), or they can be predicted (e.g. using the Jameson-Wolf antigenic index [5], matrix-based approaches [6], TEPITOPE [7], neural networks [8], OptiMer & EpiMer [9,10], ADEPT [11], Tsites [12], hydrophilicity [13], antigenic index [14] or the methods disclosed in reference 15, etc.). Other preferred fragments are (a) the N-terminal signal peptides of the Hib polypeptides of the invention, (b) the Hib polypeptides, but without their N-terminal signal peptides, (c) the Hib polypeptides, but without their N-terminal amino acid residue.

Polypeptides of the invention can be prepared in many ways e.g. by chemical synthesis (in whole or in part), by digesting longer polypeptides using proteases, by translation from RNA, by purification from cell culture (e.g. from recombinant expression), from the organism itself (e.g. after bacterial culture, or direct from patients), etc. A preferred method for production of peptides <40 amino acids long involves in vitro chemical synthesis [16,17]. Solid-phase peptide synthesis is particularly preferred, such as methods based on tBoc or Fmoc [18] chemistry. Enzymatic synthesis [19] may also be used in part or in full. As an alternative to chemical synthesis, biological synthesis may be used e.g. the polypeptides may be produced by translation. This may be carried out in vitro or in vivo. Biological methods are in general restricted to the production of polypeptides based on L-amino acids, but manipulation of translation machinery (e.g. of aminoacyl tRNA molecules) can be used to allow the introduction of D-amino acids (or of other non natural amino acids, such as iodotyrosine or methylphenylalanine, azidohomoalanine, etc.) [20]. Where D-amino acids are included, however, it is preferred to use chemical synthesis. Polypeptides of the invention may have covalent modifications at the C-terminus and/or N-terminus.

Polypeptides of the invention can take various forms (e.g. native, fusions, glycosylated, non-glycosylated, lipidated, non-lipidated, phosphorylated, non-phosphorylated, myristoylated, non-myristoylated, monomeric, multimeric, particulate, denatured, etc.).

Polypeptides of the invention are preferably provided in purified or substantially purified form i.e. substantially free from other polypeptides (e.g. free from naturally-occurring polypeptides), particularly from other Haemophilus or host cell polypeptides, and are generally at least about 50% pure (by weight), and usually at least about 90% pure i.e. less than about 50%, and more preferably less than about 10% (e.g. 5%) of a composition, is made up of other expressed polypeptides. Polypeptides of the invention are preferably H. influenzae polypeptides. Polypeptides of the invention preferably have the function indicated in Table I for the relevant sequence.

Polypeptides of the invention may be attached to a solid support. Polypeptides of the invention may comprise a detectable label (e.g. a radioactive or fluorescent label, or a biotin label).

The term “polypeptide” refers to amino acid polymers of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. Polypeptides can occur as single chains or associated chains. Polypeptides of the invention can be naturally or non-naturally glycosylated (i.e. the polypeptide has a glycosylation pattern that differs from the glycosylation pattern found in the corresponding naturally occurring polypeptide).

The invention provides polypeptides comprising a sequence —X—Y— or —Y—X—, wherein: —X— is an amino acid sequence as defined above and —Y— is not a sequence as defined above i.e. the invention provides fusion proteins. Where the N-terminus codon of a polypeptide-coding sequence is not ATG then that codon will be translated as the standard amino acid for that codon rather than as a Met, which occurs when the codon is translated as a start codon.

The invention provides a process for producing polypeptides of the invention, comprising the step of culturing a host cell of to the invention under conditions which induce polypeptide expression.

The invention provides a process for producing a polypeptide of the invention, wherein the polypeptide is synthesised in part or in whole using chemical means.

The invention provides a composition comprising two or more polypeptides of the invention.

The invention also provides a hybrid polypeptide represented by the formula NH₂-A-[—X-L-]ₙ—B—COOH, wherein X is a polypeptide of the invention as defined above, L is an optional linker amino acid sequence, A is an optional N-terminal amino acid sequence, B is an optional C-terminal amino acid sequence, and n is an integer greater than 1. The value of n is between 2 and x, and the value of x is typically 3, 4, 5, 6, 7, 8, 9 or 10. Preferably n is 2, 3 or 4; it is more preferably 2 or 3; most preferably, n=2. For each 11 instances, —X— may be the same or different. For each n instances of [—X-L-], linker amino acid sequence -L- may be present or absent. For instance, when n=2 the hybrid may be NH₂—X₁-L₁-X₂-L₂-COOH, NH₂—X₁-X₂—COOH, NH₂—X₁-L₁-X₂—COOH, NH₂—X₁-X₂-L₂-COOH, etc. Linker amino acid sequence(s) -L- will typically be short (e.g. 20 or fewer amino acids i.e. 19, 18, 17, 16, 15, 14, 13, 12, 11, 10, 9, 8, 7, 6, 5, 4, 3, 2, 1). Examples include short peptide sequences which facilitate cloning, poly-glycine linkers (i.e. Glyₙ where n=2, 3, 4, 5, 6, 7, 8, 9, 10 or more), and histidine tags (i.e. Hisₙ where n=3, 4, 5, 6, 7, 8, 9, 10 or more). Other suitable linker amino acid sequences will be apparent to those skilled in the art. -A- and —B— are optional sequences which will typically be short (e.g. 40 or fewer amino acids i.e. 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1). Examples include leader sequences to direct polypeptide trafficking, or short peptide sequences which facilitate cloning or purification (e.g. histidine tags i.e. Hisₙ where n=3, 4, 5, 6, 7, 8, 9, 10 or more). Other suitable N-terminal and C-terminal amino acid sequences will be apparent to those skilled in the art.

Various tests can be used to assess the in vivo immunogenicity of polypeptides of the invention. For example, polypeptides can be expressed recombinantly and used to screen patient sera by immunoblot. A positive reaction between the polypeptide and patient serum indicates that the patient has previously mounted an immune response to the protein in question i.e. the protein is an immunogen. This method can also be used to identify immunodominant proteins.

### Antibodies

The invention provides antibodies that bind to polypeptides of the invention. These may be polyclonal or monoclonal and may be produced by any suitable means (e.g. by recombinant expression). To increase compatibility with the human immune system, the antibodies may be chimeric or humanised [e.g. refs. 21 & 22], or fully human antibodies may be used. The antibodies may include a detectable label (e.g. for diagnostic assays). Antibodies of the invention may be attached to a solid support. Antibodies of the invention are preferably neutralising antibodies.

Monoclonal antibodies are particularly useful in identification and purification of the individual polypeptides against which they are directed. Monoclonal antibodies of the invention may also be employed as reagents in immunoassays, radioimmunoassays (RIA) or enzyme-linked immunosorbent assays (ELISA), etc. In these applications, the antibodies can be labelled with an analytically-detectable reagent such as a radioisotope, a fluorescent molecule or an enzyme. The monoclonal antibodies produced by the above method may also be used for the molecular identification and characterization (epitope mapping) of polypeptides of the invention.

Antibodies of the invention are preferably specific to Haemophilus i.e. they bind preferentially to Haemophilus bacteria relative to non-Haemophilus bacteria. More preferably, the antibodies are specific to Hib i.e. they bind preferentially to Hib bacteria relative to non-type-b H. influenzae strains.

Antibodies of the invention are preferably provided in purified or substantially purified form. Typically, the antibody will be present in a composition that is substantially free of other polypeptides e.g. where less than 90% (by weight), usually less than 60% and more usually less than 50% of the composition is made up of other polypeptides.

Antibodies of the invention can be of any isotype (e.g. IgA, IgG, IgM i.e. an α, γ or μ heavy chain), but will generally be IgG. Within the IgG isotype, antibodies may be IgG1, IgG2, IgG3 or IgG4 subclass. Antibodies of the invention may have a κ or a λ light chain.

Antibodies of the invention can take various forms, including whole antibodies, antibody fragments such as F(ab′)₂ and F(ab) fragments, Fv fragments (non-covalent heterodimers), single-chain antibodies such as single chain Fv molecules (scFv), minibodies, oligobodies, etc. The term “antibody” does not imply any particular origin, and includes antibodies obtained through non-conventional processes, such as phage display.

The invention provides a process for detecting polypeptides of the invention, comprising the steps of (a) contacting an antibody of the invention with a biological sample under conditions suitable for the formation of an antibody-antigen complexes; and (b) detecting said complexes.

The invention provides a process for detecting antibodies of the invention, comprising the steps of: (a) contacting a polypeptide of the invention with a biological sample (e.g. a blood or serum sample) under conditions suitable for the formation of an antibody-antigen complexes; and (b) detecting said complexes.

### Nucleic Acids

The invention provides nucleic acid comprising the H. influenzae nucleotide sequences disclosed in the examples. These nucleic acid sequences are the odd SEQ ID NOs between 1 and 3706.

The invention also provides nucleic acid comprising nucleotide sequences having sequence identity to the H. influenzae nucleotide sequences disclosed in the examples. Identity between sequences is preferably determined by the Smith-Waterman homology search algorithm as described above.

The invention also provides nucleic acid which can hybridize to the H. influenzae nucleic acid disclosed in the examples. Hybridization reactions can be performed under conditions of different “stringency”. Conditions that increase stringency of a hybridization reaction of widely known and published in the art [e.g. page 7.52 of reference 23]. Examples of relevant conditions include (in order of increasing stringency): incubation temperatures of 25° C., 37° C., 50° C., 55° C. and 68° C.; buffer concentrations of 10×SSC, 6×SSC, 1×SSC, 0.1×SSC (where SSC is 0.15 M NaCl and 15 mM citrate buffer) and their equivalents using other buffer systems; formamide concentrations of 0%, 25%, 50%, and 75%; incubation times from 5 minutes to 24 hours; 1, 2, or more washing steps; wash incubation times of 1, 2, or 15 minutes; and wash solutions of 6×SSC, 1×SSC, 0.1×SSC, or de-ionized water. Hybridization techniques and their optimization are well known in the art [e.g. see references 23-26, etc.].

In some embodiments, nucleic acid of the invention hybridizes to a target of the invention under low stringency conditions; in other embodiments it hybridizes under intermediate stringency conditions; in preferred embodiments, it hybridizes under high stringency conditions. An exemplary set of low stringency hybridization conditions is 50° C. and 10×SSC. An exemplary set of intermediate stringency hybridization conditions is 55° C. and 1×SSC. An exemplary set of high stringency hybridization conditions is 68° C. and 0.1×SSC.

Nucleic acid comprising fragments of these sequences are also provided. These should comprise at least n consecutive nucleotides from the H. influenzae sequences and, depending on the particular sequence, n is 10 or more (e.g. 12, 14, 15, 18, 20, 25, 30, 35, 40, 50, 60, 70, 80, 90, 100, 150, 200 or more).

The invention provides nucleic acid of formula 5′-X—Y—Z-3′, wherein: —X— is a nucleotide sequence consisting of x nucleotides; —Z— is a nucleotide sequence consisting of z nucleotides; —Y— is a nucleotide sequence consisting of either (a) a fragment of one of the odd-numbered SEQ ID NOS: 1 to 5079, or (b) the complement of (a); and said nucleic acid 5′-X—Y—Z-3′ is neither (i) a fragment of one of the odd-numbered SEQ ID NOS: 1 to 3705 nor (ii) the complement of (i). The —X— and/or —Z-moieties may comprise a promoter sequence (or its complement).

The invention also provides nucleic acid encoding the polypeptides and polypeptide fragments of the invention.

The invention includes nucleic acid comprising sequences complementary to the sequences disclosed in the sequence listing (e.g. for antisense or probing, or for use as primers), as well as the sequences in the orientation actually shown.

Nucleic acids of the invention can be used in hybridisation reactions (e.g. Northern or Southern blots, or in nucleic acid microarrays or ‘gene chips’) and amplification reactions (e.g. PCR, SDA, SSSR, LCR, TMA, NASBA, etc.) and other nucleic acid techniques.

Nucleic acid according to the invention can take various forms (e.g. single-stranded, double-stranded, vectors, primers, probes, labelled etc.). Nucleic acids of the invention may be circular or branched, but will generally be linear. Unless otherwise specified or required, any embodiment of the invention that utilizes a nucleic acid may utilize both the double-stranded form and each of two complementary single-stranded forms which make up the double-stranded form. Primers and probes are generally single-stranded, as are antisense nucleic acids.

Nucleic acids of the invention are preferably provided in purified or substantially purified form i.e. substantially free from other nucleic acids (e.g. free from naturally-occurring nucleic acids), particularly from other Haemophilus or host cell nucleic acids, generally being at least about 50% pure (by weight), and usually at least about 90% pure. Nucleic acids of the invention are preferably H. influenzae nucleic acids.

Nucleic acids of the invention may be prepared in many ways e.g. by chemical synthesis (e.g. phosphoramidite synthesis of DNA) in whole or in part, by digesting longer nucleic acids using nucleases (e.g. restriction enzymes), by joining shorter nucleic acids or nucleotides (e.g. using ligases or polymerases), from genomic or cDNA libraries, etc.

Nucleic acid of the invention may be attached to a solid support (e.g. a bead, plate, filter, film, slide, microarray support, resin, etc.). Nucleic acid of the invention may be labelled e.g. with a radioactive or fluorescent label, or a biotin label. This is particularly useful where the nucleic acid is to be used in detection techniques e.g. where the nucleic acid is a primer or as a probe.

The term “nucleic acid” includes in general means a polymeric form of nucleotides of any length, which contain deoxyribonucleotides, ribonucleotides, and/or their analogs. It includes DNA, RNA, DNA/RNA hybrids. It also includes DNA or RNA analogs, such as those containing modified backbones (e.g. peptide nucleic acids (PNAs) or phosphorothioates) or modified bases. Thus the invention includes mRNA, tRNA, rRNA, ribozymes, DNA, cDNA, recombinant nucleic acids, branched nucleic acids, plasmids, vectors, probes, primers, etc. Where nucleic acid of the invention takes the form of RNA, it may or may not have a 5′ cap.

Nucleic acids of the invention comprise Hib sequences, but they may also comprise non-Hib sequences (e.g. in nucleic acids of formula 5′-X—Y—Z-3′, as defined above). This is particularly useful for primers, which may thus comprise a first sequence complementary to a Hib nucleic acid target and a second sequence which is not complementary to the nucleic acid target. Any such non-complementary sequences in the primer are preferably 5′ to the complementary sequences. Typical non-complementary sequences comprise restriction sites or promoter sequences.

Nucleic acids of the invention can be prepared in many ways e.g. by chemical synthesis (at least in part), by digesting longer nucleic acids using nucleases (e.g. restriction enzymes), by joining shorter nucleic acids (e.g. using ligases or polymerases), from genomic or cDNA libraries, etc.

Nucleic acids of the invention may be part of a vector i.e. part of a nucleic acid construct designed for transduction/transfection of one or more cell types. Vectors may be, for example, “cloning vectors” which are designed for isolation, propagation and replication of inserted nucleotides, “expression vectors” which are designed for expression of a nucleotide sequence in a host cell, “viral vectors” which is designed to result in the production of a recombinant virus or virus-like particle, or “shuttle vectors”, which comprise the attributes of more than one type of vector. Preferred vectors are plasmids. A “host cell” includes an individual cell or cell culture which can be or has been a recipient of exogenous nucleic acid. Host cells include progeny of a single host cell, and the progeny may not necessarily be completely identical (in morphology or in total DNA complement) to the original parent cell due to natural, accidental, or deliberate mutation and/or change. Host cells include cells transfected or infected in vivo or in vitro with nucleic acid of the invention.

Where a nucleic acid is DNA, it will be appreciated that “U” in a RNA sequence will be replaced by “T” in the DNA. Similarly, where a nucleic acid is RNA, it will be appreciated that “T” in a DNA sequence will be replaced by “U” in the RNA.

The term “complement” or “complementary” when used in relation to nucleic acids refers to Watson-Crick base pairing. Thus the complement of C is G, the complement of G is C, the complement of A is T (or U), and the complement of T (or U) is A. It is also possible to use bases such as I (the purine inosine) e.g. to complement pyrimidines (C or T). The terms also imply a direction—the complement of 5′-ACAGT-3′ is 5′-ACTGT-3′ rather than 5′-TGTCA-3′.

Nucleic acids of the invention can be used, for example: to produce polypeptides; as hybridization probes for the detection of nucleic acid in biological samples; to generate additional copies of the nucleic acids; to generate ribozymes or antisense oligonucleotides; as single-stranded DNA primers or probes; or as triple-strand forming oligonucleotides.

The invention provides a process for producing nucleic acid of the invention, wherein the nucleic acid is synthesised in part or in whole using chemical means.

The invention provides vectors comprising nucleotide sequences of the invention (e.g. cloning or expression vectors) and host cells transformed with such vectors.

The invention also provides a kit comprising primers (e.g. PCR primers) for amplifying a template sequence contained within a Haemophilus bacterium (e.g. H. influenzae) nucleic acid sequence, the kit comprising a first primer and a second primer, wherein the first primer is substantially complementary to said template sequence and the second primer is substantially complementary to a complement of said template sequence, wherein the parts of said primers which have substantial complementarity define the termini of the template sequence to be amplified. The first primer and/or the second primer may include a detectable label (e.g. a fluorescent label).

The invention also provides a kit comprising first and second single-stranded oligonucleotides which allow amplification of a Haemophilus template nucleic acid sequence contained in a single- or double-stranded nucleic acid (or mixture thereof), wherein: (a) the first oligonucleotide comprises a primer sequence which is substantially complementary to said template nucleic acid sequence; (b) the second oligonucleotide comprises a primer sequence which is substantially complementary to the complement of said template nucleic acid sequence; (c) the first oligonucleotide and/or the second oligonucleotide comprise(s) sequence which is not complementary to said template nucleic acid; and (d) said primer sequences define the termini of the template sequence to be amplified. The non-complementary sequence(s) of feature (c) are preferably upstream of (i.e. 5′ to) the primer sequences. One or both of these (c) sequences may comprise a restriction site [e.g. ref. 27] or a promoter sequence [e.g. 28]. The first oligonucleotide and/or the second oligonucleotide may include a detectable label (e.g. a fluorescent label).

The template sequence may be any part of a genome sequence e.g. of SEQ ID NO:3707.

The invention provides a process for detecting nucleic acid of the invention, comprising the steps of (a) contacting a nucleic probe according to the invention with a biological sample under hybridising conditions to form duplexes; and (b) detecting said duplexes.

The invention provides a process for detecting H. influenzae in a biological sample (e.g. blood), comprising the step of contacting nucleic acid according to the invention with the biological sample under hybridising conditions. The process may involve nucleic acid amplification (e.g. PCR, SDA, SSSR, LCR, TMA, NASBA, etc.) or hybridisation (e.g. microarrays, blots, hybridisation with a probe in solution etc.). PCR detection of H. influenzae in clinical samples has been reported [e.g. see refs. 29 & 30]. Clinical assays based on nucleic acid are described in general in ref. 31.

The invention provides a process for preparing a fragment of a target sequence, wherein the fragment is prepared by extension of a nucleic acid primer. The target sequence and/or the primer are nucleic acids of the invention. The primer extension reaction may involve nucleic acid amplification (e.g. PCR, SDA, SSSR, LCR, TMA, NASBA, etc.).

Nucleic acid amplification according to the invention may be quantitative and/or real-time.

For certain embodiments of the invention, nucleic acids are preferably at least 7 nucleotides in length (e.g. 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 90, 100, 110, 120, 130, 140, 150, 160, 170, 180, 190, 200, 225, 250, 275, 300 nucleotides or longer).

For certain embodiments of the invention, nucleic acids are preferably at most 500 nucleotides in length (e.g. 450, 400, 350, 300, 250, 200, 150, 140, 130, 120, 110, 100, 90, 80, 75, 70, 65, 60, 55, 50, 45, 40, 39, 38, 37, 36, 35, 34, 33, 32, 31, 30, 29, 28, 27, 26, 25, 24, 23, 22, 21, 20, 19, 18, 17, 16, 15 nucleotides or shorter).

Primers and probes of the invention, and other nucleic acids used for hybridization, are preferably between 10 and 30 nucleotides in length (e.g. 10, 11, 12, 13, 14, 15, 16, 17, 18, 19, 20, 21, 22, 23, 24, 25, 26, 27, 28, 29, or 30 nucleotides).

### Pharmaceutical Compositions

The invention provides compositions comprising: (a) polypeptide, antibody, and/or nucleic acid of the invention; and (b) a pharmaceutically acceptable carrier. These compositions may be suitable as immunogenic compositions, for instance, or as diagnostic reagents, or as vaccines. Vaccines according to the invention may either be prophylactic (i.e. to prevent infection) or therapeutic (i.e. to treat infection), but will typically be prophylactic.

A ‘pharmaceutically acceptable carriers’ includes any carrier that does not itself induce the production of antibodies harmful to the individual receiving the composition. Suitable carriers are typically large, slowly metabolised macromolecules such as proteins, polysaccharides, polylactic acids, polyglycolic acids, polymeric amino acids, amino acid copolymers, sucrose, trehalose, lactose, and lipid aggregates (such as oil droplets or liposomes). Such carriers are well known to those of ordinary skill in the art. The vaccines may also contain diluents, such as water, saline, glycerol, etc. Additionally, auxiliary substances, such as wetting or emulsifying agents, pH buffering substances, and the like, may be present. Sterile pyrogen-free, phosphate-buffered physiologic saline is a typical carrier. A thorough discussion of pharmaceutically acceptable excipients is available in ref. 142.

Compositions of the invention may include an antimicrobial, particularly if packaged in a multiple dose format.

Compositions of the invention may comprise detergent e.g. a Tween (polysorbate), such as Tween 80. Detergents are generally present at low levels e.g. <0.01%.

Compositions of the invention may include sodium salts (e.g. sodium chloride) to give tonicity. A concentration of 10±2 mg/ml NaCl is typical.

Compositions of the invention will generally include a buffer. A phosphate buffer is typical.

Compositions of the invention may comprise a sugar alcohol (e.g. mannitol) or a disaccharide (e.g. sucrose or trehalose) e.g. at around 15-30 mg/ml (e.g. 25 mg/ml), particularly if they are to be lyophilised or if they include material which has been reconstituted from lyophilised material. The pH of a composition for lyophilisation may be adjusted to around 6.1 prior to lyophilisation.

Polypeptides of the invention may be administered in conjunction with other immunoregulatory agents. In particular, compositions will usually include a vaccine adjuvant. Adjuvants which may be used in compositions of the invention include, but are not limited to:

### A. Mineral-Containing Compositions

Mineral containing compositions suitable for use as adjuvants in the invention include mineral salts, such as aluminium salts and calcium salts. The invention includes mineral salts such as hydroxides (e.g. oxyhydroxides), phosphates (e.g. hydroxyphosphates, orthophosphates), sulphates, etc. [e.g. see chapters 8 & 9 of ref. 32], or mixtures of different mineral compounds, with the compounds taking any suitable form (e.g. gel, crystalline, amorphous, etc.), and with adsorption being preferred. The mineral containing compositions may also be formulated as a particle of metal salt [33].

Aluminium phosphates are particularly preferred, particularly in compositions which include a H. influenzae saccharide antigen, and a typical adjuvant is amorphous aluminium hydroxyphosphate with PO₄/Al molar ratio between 0.84 and 0.92, included at 0.6 mg Al³⁺/ml. Adsorption with a low dose of aluminium phosphate may be used e.g. between 50 and 100 μg Al³⁺ per conjugate per dose. Where there is more than one conjugate in a composition, not all conjugates need to be adsorbed.

### B. Oil Emulsions

Oil emulsion compositions suitable for use as adjuvants in the invention include squalene-water emulsions, such as MF59 [Chapter 10 of ref. 32; see also ref. 34] (5% Squalene, 0.5% Tween 80, and 0.5% Span 85, formulated into submicron particles using a microfluidizer). Complete Freund's adjuvant (CFA) and incomplete Freund's adjuvant (IFA) may also be used.

### C. Saponin Formulations [Chapter 22 of Ref 32]

Saponin formulations may also be used as adjuvants in the invention. Saponins are a heterologous group of sterol glycosides and triterpenoid glycosides that are found in the bark, leaves, stems, roots and even flowers of a wide range of plant species. Saponin from the bark of the Quillaia saponaria Molina tree have been widely studied as adjuvants. Saponin can also be commercially obtained from Smilax ornata (sarsaprilla), Gypsophilla paniculata (brides veil), and Saponaria officianalis (soap root). Saponin adjuvant formulations include purified formulations, such as QS21, as well as lipid formulations, such as ISCOMs. QS21 is marketed as Stimulon™.

Saponin compositions have been purified using HPLC and RP-HPLC. Specific purified fractions using these techniques have been identified, including QS7, QS17, QS18, QS21, QH-A, QH-B and QH-C. Preferably, the saponin is QS21. A method of production of QS21 is disclosed in ref. 35. Saponin formulations may also comprise a sterol, such as cholesterol[36].

Combinations of saponins and cholesterols can be used to form unique particles called immunostimulating complexs (ISCOMs) [chapter 23 of ref. 32]. ISCOMs typically also include a phospholipid such as phosphatidylethanolamine or phosphatidylcholine. Any known saponin can be used in ISCOMs. Preferably, the ISCOM includes one or more of QuilA, QHA & QHC. ISCOMs are further described in refs. 36-38. Optionally, the ISCOMS may be devoid of additional detergent [39].

A review of the development of saponin based adjuvants can be found in refs. 40 & 41.

### D. Virosomes and Virus-Like Particles

Virosomes and virus-like particles (VLPs) can also be used as adjuvants in the invention. These structures generally contain one or more proteins from a virus optionally combined or formulated with a phospholipid. They are generally non-pathogenic, non-replicating and generally do not contain any of the native viral genome. The viral proteins may be recombinantly produced or isolated from whole viruses. These viral proteins suitable for use in virosomes or VLPs include proteins derived from influenza virus (such as HA or NA), Hepatitis B virus (such as core or capsid proteins), Hepatitis E virus, measles virus, Sindbis virus, Rotavirus, Foot-and-Mouth Disease virus, Retrovirus, Norwalk virus, human Papilloma virus, HIV, RNA-phages, Qβ-phage (such as coat proteins), GA-phage, fr-phage, AP205 phage, and Ty (such as retrotransposon Ty protein pl). VLPs are discussed further in refs. 42-47. Virosomes are discussed further in, for example, ref. 48

### E. Bacterial or Microbial Derivatives

Adjuvants suitable for use in the invention include bacterial or microbial derivatives such as non-toxic derivatives of enterobacterial lipopolysaccharide (LPS), Lipid A derivatives, immunostimulatory oligonucleotides and ADP-ribosylating toxins and detoxified derivatives thereof.

Non-toxic derivatives of LPS include monophosphoryl lipid A (MPL) and 3-O-deacylated MPL (3dMPL). 3dMPL is a mixture of 3 de-O-acylated monophosphoryl lipid A with 4, 5 or 6 acylated chains. A preferred “small particle” form of 3 De-O-acylated monophosphoryl lipid A is disclosed in ref. 49. Such “small particles” of 3dMPL are small enough to be sterile filtered through a 0.22 μm membrane[49]. Other non-toxic LPS derivatives include monophosphoryl lipid A mimics, such as aminoalkyl glucosaminide phosphate derivatives e.g. RC-529 [50,51].

Lipid A derivatives include derivatives of lipid A from Escherichia coli such as OM-174. OM-174 is described for example in refs. 52 & 53.

Immunostimulatory oligonucleotides suitable for use as adjuvants in the invention include nucleotide sequences containing a CpG motif (a dinucleotide sequence containing an unmethylated cytosine linked by a phosphate bond to a guanosine). Double-stranded RNAs and oligonucleotides containing palindromic or poly(dG) sequences have also been shown to be immunostimulatory.

The CpG's can include nucleotide modifications/analogs such as phosphorothioate modifications and can be double-stranded or single-stranded. References 54, 55 and 56 disclose possible analog substitutions e.g. replacement of guanosine with 2′-deoxy-7-deazaguanosine. The adjuvant effect of CpG oligonucleotides is further discussed in refs. 57-62.

The CpG sequence may be directed to TLR9, such as the motif GTCGTT or TTCGTT [63]. The CpG sequence may be specific for inducing a Th1 immune response, such as a CpG-A ODN, or it may be more specific for inducing a B cell response, such a CpG-B ODN. CpG-A and CpG-B ODNs are discussed in refs. 64-66. Preferably, the CpG is a CpG-A ODN.

Preferably, the CpG oligonucleotide is constructed so that the 5′ end is accessible for receptor recognition. Optionally, two CpG oligonucleotide sequences may be attached at their 3′ ends to form “immunomers”. See, for example, refs. 63 & 67-69.

Bacterial ADP-ribosylating toxins and detoxified derivatives thereof may be used as adjuvants in the invention. Preferably, the protein is derived from E. coli (E. coli heat labile enterotoxin “LT”), cholera (“CT”), or pertussis (“PT”). The use of detoxified ADP-ribosylating toxins as mucosal adjuvants is described in ref. 70 and as parenteral adjuvants in ref. 71. The toxin or toxoid is preferably in the form of a holotoxin, comprising both A and B subunits. Preferably, the A subunit contains a detoxifying mutation; preferably the B subunit is not mutated. Preferably, the adjuvant is a detoxified LT mutant such as LT-K63, LT-R72, and LT-G192. The use of ADP-ribosylating toxins and detoxified derivaties thereof, particularly LT-K63 and LT-R72, as adjuvants can be found in refs. 72-79. Numerical reference for amino acid substitutions is preferably based on the alignments of the A and B subunits of ADP-ribosylating toxins set forth in ref. 80, specifically incorporated herein by reference in its entirety.

### F. Human Immunomodulators

Human immunomodulators suitable for use as adjuvants in the invention include cytokines, such as interleukins (e.g. IL-1, IL-2, IL-4, IL-5, IL-6, IL-7, IL-12 [81], etc.) [82], interferons (e.g. interferon-′y), macrophage colony stimulating factor, and tumor necrosis factor.

### G. Bioadhesives and Mucoadhesives

Bioadhesives and mucoadhesives may also be used as adjuvants in the invention. Suitable bioadhesives include esterified hyaluronic acid microspheres [83] or mucoadhesives such as cross-linked derivatives of poly(acrylic acid), polyvinyl alcohol, polyvinyl pyrollidone, polysaccharides and carboxymethylcellulose. Chitosan and derivatives thereof may also be used as adjuvants in the invention [84].

### H. Microparticles

Microparticles may also be used as adjuvants in the invention. Microparticles (i.e. a particle of ˜100 nm to ˜150 μm in diameter, more preferably ˜200 nm to ˜30 μm in diameter, and most preferably ˜500 nm to ˜10 μm in diameter) formed from materials that are biodegradable and non-toxic (e.g. a poly(α-hydroxy acid), a polyhydroxybutyric acid, a polyorthoester, a polyanhydride, a polycaprolactone, etc.), with poly(lactide-co-glycolide) are preferred, optionally treated to have a negatively-charged surface (e.g. with SDS) or a positively-charged surface (e.g. with a cationic detergent, such as CTAB).

### I. Liposomes (Chapters 13 & 14 of ref 32)

Examples of liposome formulations suitable for use as adjuvants are described in refs. 85-87.

### J. Polyoxyethylene Ether and Polyoxyethylene Ester Formulations

Adjuvants suitable for use in the invention include polyoxyethylene ethers and polyoxyethylene esters [88]. Such formulations further include polyoxyethylene sorbitan ester surfactants in combination with an octoxynol [89] as well as polyoxyethylene alkyl ethers or ester surfactants in combination with at least one additional non-ionic surfactant such as an octoxynol [90]. Preferred polyoxyethylene ethers are selected from the following group: polyoxyethylene-9-lauryl ether (laureth 9), polyoxyethylene-9-steoryl ether, polyoxytheylene-8-steoryl ether, polyoxyethylene-4-lauryl ether, polyoxyethylene-35-lauryl ether, and polyoxyethylene-23-lauryl ether.

### K. Polyphosphazene (PCPP)

PCPP formulations are described, for example, in refs. 91 and 92.

### L. Muramyl Peptides

Examples of muramyl peptides suitable for use as adjuvants in the invention include N-acetyl-muramyl-L-threonyl-D-isoglutamine (thr-MDP), N-acetyl-normuramyl-L-alanyl-D-isoglutamine (nor-MDP), and N-acetylmuramyl-L-alanyl-D-isoglutaminyl-L-alanine-2-(1′-2′-dipalmitoyl-sn-glycero-3-hydroxyphosphoryloxy)-ethylamine MTP-PE).

### M. Imidazoquinolone Compounds.

Examples of imidazoquinolone compounds suitable for use adjuvants in the invention include Imiquamod and its homologues (e.g. “Resiquimod 3M”), described further in refs. 93 and 94.

The invention may also comprise combinations of aspects of one or more of the adjuvants identified above. For example, the following adjuvant compositions may be used in the invention: (1) a saponin and an oil-in-water emulsion [95]; (2) a saponin (e.g. QS21)+a non-toxic LPS derivative (e.g. 3dMPL) [96]; (3) a saponin (e.g. QS21)+a non-toxic LPS derivative (e.g. 3dMPL)+a cholesterol; (4) a saponin (e.g. QS21)+3dMPL+IL-12 (optionally+a sterol) [97]; (5) combinations of 3dMPL with, for example, QS21 and/or oil-in-water emulsions [98]; (6) SAF, containing 10% squalane, 0.4% Tween 80™, 5% pluronic-block polymer L121, and thr-MDP, either microfluidized into a submicron emulsion or vortexed to generate a larger particle size emulsion. (7) Ribi™ adjuvant system (RAS), (Ribi Immunochem) containing 2% squalene, 0.2% Tween 80, and one or more bacterial cell wall components from the group consisting of monophosphorylipid A (MPL), trehalose dimycolate (TDM), and cell wall skeleton (CWS), preferably MPL+CWS (Detox™); and (8) one or more mineral salts (such as an aluminum salt)+a non-toxic derivative of LPS (such as 3dMPL).

Other substances that act as immunostimulating agents are disclosed in chapter 7 of ref. 32.

The use of an aluminium hydroxide or aluminium phosphate adjuvant is particularly preferred, and antigens are generally adsorbed to these salts. Calcium phosphate is another preferred adjuvant.

The pH of compositions of the invention is preferably between 6 and 8, preferably about 7. Stable pH may be maintained by the use of a buffer. Where a composition comprises an aluminium hydroxide salt, it is preferred to use a histidine buffer [99]. The composition may be sterile and/or pyrogen-free. Compositions of the invention may be isotonic with respect to humans.

Compositions may be presented in vials, or they may be presented in ready-filled syringes. The syringes may be supplied with or without needles. A syringe will include a single dose of the composition, whereas a vial may include a single dose or multiple doses. Injectable compositions will usually be liquid solutions or suspensions. Alternatively, they may be presented in solid form (e.g. freeze-dried) for solution or suspension in liquid vehicles prior to injection.

Compositions of the invention may be packaged in unit dose form or in multiple dose form. For multiple dose forms, vials are preferred to pre-filled syringes. Effective dosage volumes can be routinely established, but a typical human dose of the composition for injection has a volume of 0.5 ml.

Where a composition of the invention is to be prepared extemporaneously prior to use (e.g. where a component is presented in lyophilised form) and is presented as a kit, the kit may comprise two vials, or it may comprise one ready-filled syringe and one vial, with the contents of the syringe being used to reactivate the contents of the vial prior to injection.

Immunogenic compositions used as vaccines comprise an immunologically effective amount of antigen(s), as well as any other components, as needed. By ‘immunologically effective amount’, it is meant that the administration of that amount to an individual, either in a single dose or as part of a series, is effective for treatment or prevention. This amount varies depending upon the health and physical condition of the individual to be treated, age, the taxonomic group of individual to be treated (e.g. non-human primate, primate, etc.), the capacity of the individual's immune system to synthesise antibodies, the degree of protection desired, the formulation of the vaccine, the treating doctor's assessment of the medical situation, and other relevant factors. It is expected that the amount will fall in a relatively broad range that can be determined through routine trials, and a typical quantity of each meningococcal saccharide antigen per dose is between 1 μg and 10 mg per antigen.

### Pharmaceutical Uses

The invention also provides a method of treating a patient, comprising administering to the patient a therapeutically effective amount of a composition of the invention. The patient may either be at risk from the disease themselves or may be a pregnant woman (‘maternal immunisation’).

The invention provides nucleic acid, polypeptide, or antibody of the invention for use as medicaments (e.g. as immunogenic compositions or as vaccines) or as diagnostic reagents. It also provides the use of nucleic acid, polypeptide, or antibody of the invention in the manufacture of (i) a medicament for treating or preventing disease and/or infection caused by H. influenzae; (ii) a diagnostic reagent for detecting the presence of H. influenzae or of antibodies raised against H. influenzae; and/or (iii) a reagent which can raise antibodies against H. influenzae. Said H. influenzae serotype or strain, but is preferably type b H. influenzae. Said disease may be, for instance, otitis media, bronchitis, conjunctivitis, sinusitis, a urinary tract infection, pneumonia, bacteremia, septic arthritis, epiglottitis, pneumonia, empyema, pericarditis, cellulitis, osteomyelitis or meningitis. The invention is particularly useful for preventing bacterial meningitis caused by Hib.

The patient is preferably a human. Where the vaccine is for prophylactic use, the human is preferably a child (e.g. a toddler or infant); where the vaccine is for therapeutic use, the human is preferably an adult. A vaccine intended for children may also be administered to adults e.g. to assess safety, dosage, immunogenicity, etc.

One way of checking efficacy of therapeutic treatment involves monitoring Hib infection after administration of the composition of the invention. One way of checking efficacy of prophylactic treatment involves monitoring immune responses against an administered polypeptide after administration. Immunogenicity of compositions of the invention can be determined by administering them to test subjects (e.g. children 12-16 months age, or animal models [e.g. a chinchilla model [Error! Bookmark not defined.]) and then determining standard parameters including ELISA titres (GMT) of IgG. These immune responses will generally be determined around 4 weeks after administration of the composition, and compared to values determined before administration of the composition. Where more than one dose of the composition is administered, more than one post-administration determination may be made.

Administration of polypeptide antigens is a preferred method of treatment for inducing immunity. Administration of antibodies of the invention is another preferred method of treatment. This method of passive immunisation is particularly useful for newborn children or for pregnant women. This method will typically use monoclonal antibodies, which will be humanised or fully human.

Compositions of the invention will generally be administered directly to a patient. Direct delivery may be accomplished by parenteral injection (e.g. subcutaneously, intraperitoneally, intravenously, intramuscularly, or to the interstitial space of a tissue), or by rectal, oral, vaginal, topical, transdermal, intranasal, sublingual, ocular, aural, pulmonary or other mucosal administration. Intramuscular administration to the thigh or the upper arm is preferred. Injection may be via a needle (e.g. a hypodermic needle), but needle-free injection may alternatively be used. A typical intramuscular dose is 0.5 ml.

The invention may be used to elicit systemic and/or mucosal immunity.

Dosage treatment can be a single dose schedule or a multiple dose schedule. Multiple doses may be used in a primary immunisation schedule and/or in a booster immunisation schedule. A primary dose schedule may be followed by a booster dose schedule. Suitable timing between priming doses (e.g. between 4-16 weeks), and between priming and boosting, can be routinely determined.

Bacterial infections affect various areas of the body and so compositions may be prepared in various forms. For example, the compositions may be prepared as injectables, either as liquid solutions or suspensions. Solid forms suitable for solution in, or suspension in, liquid vehicles prior to injection can also be prepared (e.g. a lyophilised composition). The composition may be prepared for topical administration e.g. as an ointment, cream or powder. The composition be prepared for oral administration e.g. as a tablet or capsule, or as a syrup (optionally flavoured). The composition may be prepared for pulmonary administration e.g. as an inhaler, using a fine powder or a spray. The composition may be prepared as a suppository or pessary. The composition may be prepared for nasal, aural or ocular administration e.g. as spray, drops, gel or powder [e.g. refs 100 & 101].

### Further Antigenic Components of Compositions of the Invention

The invention also provides a composition comprising a polypeptide or the invention and one or more of the following further antigens:
a saccharide antigen from N. meningitidis serogroup A, C, W135 and/or Y (preferably all four), such as the oligosaccharide disclosed in ref. 102 from serogroup C [see also ref. 103] or the oligosaccharides of ref. 104.
a saccharide antigen from Streptococcus pneumoniae [e.g. 105, 106, 107].
an antigen from hepatitis A virus, such as inactivated virus [e.g. 108, 109].
an antigen from hepatitis B virus, such as the surface and/or core antigens [e.g. 109, 110].
a diphtheria antigen, such as a diphtheria toxoid [e.g. chapter 3 of ref. 111] e.g. the CRM₁₉₇ mutant [e.g. 112].
a tetanus antigen, such as a tetanus toxoid [e.g. chapter 4 of ref. 111].
an antigen from Bordetella pertussis, such as pertussis holotoxin (PT) and filamentous haemagglutinin (FHA) from B. pertussis, optionally also in combination with pertactin and/or agglutinogens 2 and 3 [e.g. refs. 113 & 114].
a saccharide antigen from Haemophilus influenzae B [e.g. 103].
polio antigen(s) [e.g. 115, 116] such as IPV.
measles, mumps and/or rubella antigens [e.g. chapters 9, 10 & 11 of ref. 111].
influenza antigen(s) [e.g. chapter 19 of ref. 111], such as the haemagglutinin and/or neuraminidase surface proteins.
an antigen from Moraxella catarrhalis [e.g. 117].
an protein antigen from Streptococcus agalactiae (group B streptococcus) [e.g. 118, 119].
a saccharide antigen from Streptococcus agalactiae (group B streptococcus).
an antigen from Streptococcus pyogenes (group A streptococcus) [e.g. 119, 120, 121].
an antigen from Staphylococcus aureus [e.g. 122].

The composition may comprise one or more of these further antigens.

Toxic protein antigens may be detoxified where necessary (e.g. detoxification of pertussis toxin by chemical and/or genetic means [114]).

Where a diphtheria antigen is included in the composition it is preferred also to include tetanus antigen and pertussis antigens. Similarly, where a tetanus antigen is included it is preferred also to include diphtheria and pertussis antigens. Similarly, where a pertussis antigen is included it is preferred also to include diphtheria and tetanus antigens. DTP combinations are thus preferred.

Saccharide antigens are preferably in the form of conjugates. Carrier proteins for the conjugates include bacterial toxins (such as diphtheria toxoid or tetanus toxoid), the N. Meningitidis outer membrane protein [123], synthetic peptides [124,125], heat shock proteins [126,127], pertussis proteins [128,129], protein D from H. influenzae [130,131], cytokines [132], lymphokines [132], H. influenzae proteins, hormones [132], growth factors [132], toxin A or B from C. difficile [133], iron-uptake proteins [134], artificial proteins comprising multiple human CD4+ T cell epitopes from various pathogen-derived antigens [135] such as the N19 protein [136], pneumococcal surface protein PspA [137], pneumolysin [138], etc. A preferred carrier protein is the CRM197 protein [139].

Antigens in the composition will typically be present at a concentration of at least 1 μg/ml each. In general, the concentration of any given antigen will be sufficient to elicit an immune response against that antigen.

As an alternative to using proteins antigens in the immunogenic compositions of the invention, nucleic acid (preferably DNA e.g. in the form of a plasmid) encoding the antigen may be used.

Antigens are preferably adsorbed to an aluminium salt.

### Screening Methods

The invention provides a process for determining whether a test compound binds to a polypeptide of the invention. If a test compound binds to a polypeptide of the invention and this binding inhibits the life cycle of the H. influenzae bacterium, then the test compound can be used as an antibiotic or as a lead compound for the design of antibiotics. The process will typically comprise the steps of contacting a test compound with a polypeptide of the invention, and determining whether the test compound binds to said polypeptide. Preferred polypeptides of the invention for use in these processes are enzymes (e.g. tRNA synthetases), membrane transporters and ribosomal polypeptides. Suitable test compounds include polypeptides, polypeptides, carbohydrates, lipids, nucleic acids (e.g. DNA, RNA, and modified forms thereof), as well as small organic compounds (e.g. MW between 200 and 2000 Da). The test compounds may be provided individually, but will typically be part of a library (e.g. a combinatorial library). Methods for detecting a binding interaction include NMR, filter-binding assays, gel-retardation assays, displacement assays, surface plasmon resonance, reverse two-hybrid etc. A compound which binds to a polypeptide of the invention can be tested for antibiotic activity by contacting the compound with Hib bacteria and then monitoring for inhibition of growth. The invention also provides a compound identified using these methods.

Preferably, the process comprises the steps of: (a) contacting a polypeptide of the invention with one or more candidate compounds to give a mixture; (b) incubating the mixture to allow polypeptide and the candidate compound(s) to interact; and (c) assessing whether the candidate compound binds to the polypeptide or modulates its activity.

Once a candidate compound has been identified in vitro as a compound that binds to a polypeptide of the invention then it may be desirable to perform further experiments to confirm the in vivo function of the compound in inhibiting bacterial growth and/or survival. Thus the method comprise the further step of contacting the compound with a Hib bacterium and assessing its effect.

The polypeptide used in the screening process may be free in solution, affixed to a solid support, located on a cell surface or located intracellularly. Preferably, the binding of a candidate compound to the polypeptide is detected by means of a label directly or indirectly associated with the candidate compound. The label may be a fluorophore, radioisotope, or other detectable label.

### General

The invention provides a computer-readable medium (e.g. a floppy disk, a hard disk, a CD-ROM, a DVD etc.) and/or a computer memory and/or a computer database containing one or more of the sequences in the sequence listing.

The term “comprising” encompasses “including” as well as “consisting” e.g. a composition “comprising” X may consist exclusively of X or may include something additional e.g. X+Y.

The term “about” in relation to a numerical value x means, for example, x±10%.

The word “substantially” does not exclude “completely” e.g. a composition which is “substantially free” from Y may be completely free from Y. Where necessary, the word “substantially” may be omitted from the definition of the invention.

The N-terminus residues in the amino acid sequences in the sequence listing are given as the amino acid encoded by the first codon in the corresponding nucleotide sequence. Where the first codon is not ATG, it will be understood that it will be translated as methionine when the codon is a start codon, but will be translated as the indicated non-Met amino acid when the sequence is at the C-terminus of a fusion partner. The invention specifically discloses and encompasses each of the amino acid sequences of the sequence listing having a N-terminus methionine residue (e.g. a formyl-methionine residue) in place of any indicated non-Met residue.

As indicated in the above text, nucleic acids and polypeptides of the invention may include sequences that:
(a) are identical (i.e. 100% identical) to the sequences disclosed in the sequence listing;
(b) share sequence identity with the sequences disclosed in the sequence listing;
(c) have 1, 2, 3, 4, 5, 6, 7, 8, 9 or 10 single nucleotide or amino acid alterations (deletions, insertions, substitutions), which may be at separate locations or may be contiguous, as compared to the sequences of (a) or (b); and
(d) when aligned with a particular sequence from the sequence listing using a pairwise alignment algorithm, a moving window of x monomers (amino acids or nucleotides) moving from start (N-terminus or 5′) to end (C-terminus of 3′), such that for an alignment that extends to p monomers (where p>x) there are p−x+1 such windows, each window has at least xy identical aligned monomers, where: x is selected from 20, 25, 30, 35, 40, 45, 50, 60, 70, 80, 90, 100, 150, 200; y is selected from 0.50, 0.60, 0.70, 0.75, 0.80, 0.85, 0.90, 0.91, 0.92, 0.93, 0.94, 0.95, 0.96, 0.97, 0.98, 0.99; and if xy is is not an integer then it is rounded up to the nearest integer. The preferred pairwise alignment algorithm is the Needleman-Wunsch global alignment algorithm [140], using default parameters (e.g. with Gap opening penalty=10.0, and with Gap extension penalty=0.5, using the EBLOSUM62 scoring matrix). This algorithm is conveniently implemented in the needle tool in the EMBOSS package [141].

The nucleic acids and polypeptides of the invention may additionally have further sequences to the N-terminus/5′ and/or C-terminus/3′ of these sequences (a) to (d).

The practice of the present invention will employ, unless otherwise indicated, conventional methods of chemistry, biochemistry, molecular biology, immunology and pharmacology, within the skill of the art. Such techniques are explained fully in the literature. See, e.g., references 142-149, etc.

### BRIEF DESCRIPTION OF DRAWINGS

There are no drawings.

### MODES FOR CARRYING OUT THE INVENTION

Genome sequencing has been carried out on a Hib isolate (strain HK707). A genome sequence is given as SEQ ID NO: 3707. A total of 1853 coding sequences were identified in this genome, and these are given in the sequence listing together with their inferred translation products. Annotation of these polypeptide sequences is given in Table I. From the sequenced material, polypeptide-coding sequences of particular interest were selected for further work, with particular attention to immunogenic proteins for vaccine development.

### Lipoproteins

Of the 1853 encoded sequences, the following 32 were identified as lipoproteins: HIB0150; HIB0158; HIB0164; HIB0233; HIB0374; HIB0382; HIB0426; HIB0469; HIB0723; HIB0733; HIB0734; HIB0740; HIB0750; HIB0761; HIB0838; HIB0971; HIB0984; HIB1015; HIB1027; HIB1038; HIB1160; HIB1253; HIB1255; HIB1349; HIB1384; HIB1407; HIB1557; HIB1564; HIB1654; HIB1655; HIB1679; and HIB1722. Lipoproteins are surface-exposed and, as such, they represent accessible immunological targets e.g. for diagnostic and for immunisation purposes. Moreover, it has been found in B. burgdorferi [150] that OspA protein is immunogenic in a lipidated form but is non-immunogenic in a non-lipidated form, and the authors concluded that post-translational lipid attachment is a critical determinant of OspA immunogenicity.

HIB1027 and HIB1255 show similarity to proteins ‘287’ and ‘741’ from Neisseria meningitidis, which are both candidate proteins for use in vaccines. HIB1027 and HIB1255 align as follows (T-COFFEE version 2.08):

```
HIB1027 MKLNLSKFSLTILTTVMLASCGSGGGDNTQLVSPPKPAEQSKPAEQSKPA
HIB1255 MKITFTRSLLATAVMVGLTACGSGGGNG----------------------
                **:.::: *: . * *::******:.
HIB1027 DQSKSVEQSILGMPERLPTNTGLAFSIKTEDEGNINTIKNEQELIATNNF
HIB1255 -MNNNTTSQVTG-------KTGAMYTVSLTNDNKIGTVTKTP--LNNSDI
                    .:.. ..: * :** :::. ::.:*.*:.: : ..::
HIB1027 ASINVDGKNIPIDFKLEPSQGWTKEGAFIEELNLAPHICCGKYT-----D
HIB1255 NSLNLD-------------------SASTQRINEAMNKISEEFKSKTGLD
                  *:*:* .* :.:* * : . ::. *
HIB1027 VRFGAIASHSFGQDDILFYNGNPSNSVPESGEVTYKGESIMADKGNSVFG
HIB1255 VVTGA-AIVSNGEKFHIIYNGNPTETMPVQGSIHYKGSAVLGGWSADAPL
                * ** * * *:. ::*****::::* .*.: ***.:::.. . ..
HIB1027 GYRKGTSEFKVNFGDKKLSGSLNVDSPKYDVESGESKFNKVKVDINADIS
HIB1255 SIEKGTSQFDVNFADSTLTGTLNV--PNFSL-----------VSISASVS
                . .****:*.***.*..*:*:*** *::.: *.*.*.:*
HIB1027 GNKFYGSAKSSSFVSEAVSEGKFYGDGAKELGGMVKAKDNSWVGAYGAKA
HIB1255 GNSFSGRATSPDAPDGAVVEGKFYGKDALGLSGMLKT--NTFTDNFGGAG
                **.* * *.*.. . ** ******..* *.**:*: *::.. :*. .
HIB1027 Q-----------
HIB1255 IFSAIDETKITQ
```

Lipoproteins generally have a N-terminal cysteine residue, to which the lipid is covalently attached. To prepare the lipoprotein via bacterial expression generally requires a suitable N-terminal signal peptide to direct lipidation by diacylglyceryl transferase, followed by cleavage by lipoprotein-specific (type II) SPase. Lipoproteins of the invention will thus typically have a N-terminal cysteine, but will be products of post-translational modification of a nascent protein which has the usual N-terminal methionine. Such lipoproteins may be associated with a lipid bilayer and may be solubilised with detergent.

Processing and lipidation of the HIB1027 sequence will give the following mature sequence (SEQ ID NO: 3708):

CGSGGGDNTQLVSPPKPAEQSKPAEQSKPADQSKSVEQSILGMPERL
PTNTGLAFSIKTEDEGNINTIKNEQELIATNNFASINVDGKNIPIDF
KLEPSQGWTKEGAFIEELNLAPHICCGKYTDVREGATASHSFGQDDI
LFYNGNPSNSVPESGEVTYKGESIMADKGNSVEGGYRKGTSEFKVNF
GDKKLSGSLNVDSPKYDVESGESKFNKVKVDINADISGNKFYGSAKS
SSFVSEAVSEGKEYGDGAKELGGMVKAKDNSWVGAYGAKAQ

Processing and lipidation of the HIB1255 sequence will give the following mature sequence (SEQ ID NO: 3709):

CGSGGGNGMNNNTTSQVTGKTGAMYTVSLTNDNKIGTVTKTPLNNSD
INSLNLDSASTORINEAMNKISEEFKSKTGLDVVTGAAIVSNGEKFH
IIYNGNPTETMPVQGSIHYKGSAVLGGWSADAPLSIEKGTSQFDVNF
ADSTLTGTLNVPNFSLVSISASVSGNSFSGRATSPDAPDGAVVEGKF
YGKDALGLSGMLKTNTFTDNFGGAGIFSATDETKITQ

Compared to the genomes of H. influenzae Rd and of a non-typeable H. influenzae, HIB1255 is part of an insert, between homologous sequences hil192 and hil193. This 2.3 kb insert contains three coding sequences and has a GC content of 32.4%.

Their similarity to N. meningitidis vaccine antigens, and their absence in non-pathogenic strains, suggests that HIB1027 and HIB1255 are useful Hib immunogens.

### Inner and Outer Membranes

As H. influenzae is a Grain-negative bacterium, its cell wall includes an outer membrane. Of the 1853 coding sequences, the following 17 were identified as being located in this outer membrane: HIB0124; HIB0374; HIB0382; HIB0394; HIB0426; HIB0733; HIB0734; HIB0965; HIB0966; HIB1224; HIB1561; HIB1564; HIB1566; HIB1654; HIB1665; HIB1679; and HIB1835. Outer membrane proteins (OMPs) are surface-exposed and, as such, they represent accessible immunological targets e.g. for diagnostic and for immunisation purposes. OMPs are often invasins, adhesins, etc. which, if blocked, offers a means of preventing bacterial infection.

As H. influenzae is a Gram-negative bacterium, it also has an inner membrane. Of the 1853 coding sequences, the following pair were identified as being located in the inner membrane: HIB1055; HIB1086. Inner membrane proteins represent useful immunological targets e.g. for diagnostic and for immunisation purposes.

### Periplasm

As H. influenzae is a Gram-negative bacterium, it has a periplasm between its cell cytoplasmic membrane and its outer membrane. Of the 1853 coding sequences, the following 16 were identified as being located in the periplasm: HIB0089; HIB0288; HIB0338; HIB0341; HIB0525; HIB0999; HIB1088; HIB1141; HIB1172; HIB1185; HIB1238; HIB1334; HIB1576; HIB1583; HIB1709; and HIB1761. Periplasmic proteins represent useful immunological targets e.g. for diagnostic and for immunisation purposes.

It will be understood that the invention has been described by way of example only and modifications may be made whilst remaining within the scope and spirit of the invention.

TABLE I
Annotations

| HIB | Annotation |
|---|---|
| 0001 | Glyceraldehyde 3-phosphate dehydrogenase, C-terminal domain |
| 0002 | FadD (LACS) [6.2.1.3] |
| 0003 | Protein |
| 0004 | predicted metal-dependent hydrolase |
| 0005 | formate dehydrogenase family accessory protein FdhD (fdhD) |
| 0006 | 1.2.1.2 [1.2.1.2] |
| 0007 | formate dehydrogenase, alpha subunit [1.2.1.2] |
| 0008 | formate dehydrogenase, beta subunit (FdxH) [1.2.1.2] |
| 0009 | formate dehydrogenase, gamma subunit [1.2.1.2] |
| 0010 | formate dehydrogenase accessory protein FdhE (fdhE) |
| 0011 | ribosomal-protein-alanine acetyltransferase (rimI) [2.3.1.128] |
| 0012 | DNA polymerase III, psi subunit (holD) [2.7.7.7] |
| 0013 | Ribosomal RNA small subunit methyltransferase C (rRNA(guanine-N(2)-)-methyltransferase) (16S rRNA |
| | m2G1207methyltransferase) (AE005668) [2.1.1.52] |
| 0014 | GTP-binding protein Era (era) |
| 0015 | ribonuclease III (rnc) [3.1.26.3] |
| 0016 | Signal peptidase I (SPase I) (Leader peptidase I) (lepB) [3.4.21.89] |
| 0017 | GTP-binding protein LepA (lepA) |
| 0018 | Protein (pfl) [2.3.1.54] |
| 0019 | uracil-DNA glycosylase (ung) [3.2.2.—] |
| 0020 | tRNA-i(6)A37 thiotransferase enzyme MiaB (miaB) |
| 0021 | 2-oxoglutarate/malate translocator (SODiT1) |
| 0022 | 2.7.7.61 (citG) [2.7.7.61] |
| 0023 | citrate lyase, alpha subunit (citF) [2.8.3.10] |
| 0024 | citrate lyase, beta subunit (citE) [4.1.3.6] |
| 0025 | citrate lyase acyl carrier protein (citD) |
| 0026 | citrate lyase ligase (citC) [6.2.1.22] |
| 0027 | lipoic acid synthetase (lipA) |
| 0028 | lipoate-protein ligase B (lipB) |
| 0029 | UPF0250 protein |
| 0030 | Penicillin-binding protein 5 precursor (D-alanyl-D-alaninecarboxypeptidase fraction A) (DD-peptidase)(DD- |
| | carboxypeptidase) (PBP-5) (dacA) [3.4.16.4] |
| 0031 | RlpA-like protein precursor (rlpA) |
| 0032 | rod shape-determining protein RodA (rodA) |
| 0033 | Penicillin-binding protein 2 (PBP-2) (pbp2) |
| 0034 | conserved hypothetical protein TIGR00246 |
| 0035 | iojap-related protein |
| 0036 | Hypothetical membrane protein |
| 0037 | ABC transporter, ATP-binding protein |
| 0038 | rod shape-determining protein (mreB) |
| 0039 | rod shape-determining protein MreC (mreC) |
| 0040 | rod shape-determining protein MreD (mreD) |
| 0041 | conserved hypothetical protein TIGR01619 |
| 0042 | exodeoxyribonuclease III (xth) [3.1.11.2] |
| 0043 | pseudouridine synthase Rlu family protein, TIGR01621 |
| 0044 | conserved hypothetical protein TIGR01620 |
| 0045 | Integral membrane protein |
| 0046 | conserved hypothetical protein YtfJ-family, TIGR01626 |
| 0047 | PhnA protein homolog (phnA) |
| 0048 | IS103 orf (orfB) |
| 0049 | glutamate--cysteine ligase, putative/amino acid ligase, putative |
| 0050 | membrane protein, TerC family |
| 0051 | excinuclease ABC, C subunit (uvrC) |
| 0052 | 3-deoxy-D-manno-octulosonate cytidylyltransferase (kdsB) [2.7.7.38] |
| 0053 | tetraacyldisaccharide 4′-kinase (lpxK) [2.7.1.130] |
| 0054 | lipid A export ATP-binding/permease protein MsbA (msbA) |
| 0055 | DNA internalization-related competence protein ComEC/Rec2 |
| 0056 | DnaK suppressor protein homolog (dksA) |
| 0057 | PcnB (pcnB) [2.7.7.19] |
| 0058 | 2-amino-4-hydroxy-6-hydroxymethyldihydropteridine pyrophosphokinase (folK) [2.7.6.3] |
| 0059 | conserved hypothetical protein TIGR00150 |
| 0060 | N-acetylmuramoyl-L-alanine amidase [3.5.1.28] |
| 0061 | DNA mismatch repair protein mutL (mutL) |
| 0062 | tRNA delta(2)-isopentenylpyrophosphate transferase (miaA) [2.5.1.8] |
| 0063 | Glutamate-ammonia-ligase adenylyltransferase (Glutamine-synthetase adenylyltransferase) (ATASE) (glnE) |
| | [2.7.7.42] |
| 0064 | DNA repair protein RecN (recN) |
| 0065 | Predicted kinase [2.7.1.23] |
| 0066 | heat shock protein B25.3 homolog (grpE) |
| 0067 | nucleotidyltransferase domain protein |
| 0068 | nucleotidyltransferase substrate binding protein, HI0074 family subfamily |
| 0069 | Anaerobic ribonucleoside-triphosphate reductase (nrdD) [1.17.4.2] |
| 0070 | acyl-CoA thioesterase II (tesB) [3.1.2.—] |
| 0071 | cysteinyl-tRNA synthetase (cysS) [6.1.1.16] |
| 0072 | Peptidyl-prolyl cis-trans isomerase B (PPIase B)(Rotamase B) (ppiB) [5.2.1.8] |
| 0073 | Mg-dependent DNase [3.1.21.—] |
| 0074 | 5-METHYLTETRAHYDROPTEROYLTRIGLUTAMATE--HOMOCYSTEINE METHYLTRANSFERASE |
| | (METHIONINE SYNTHASE, VITAMIN-B12 INDEPENDENT ISOZYME)(COBALAMIN-INDEPENDENT |
| | METHIONINE SYNTHASE) |
| 0075 | thioredoxin (trx) |
| 0076 | 2-hydroxyacid dehydrogenase homolog (ldhA) [1.1.1.—] |
| 0077 | cystathionine gamma-lyase homolog (metB) [2.5.1.48] |
| 0078 | threonine synthase (thrC) [4.2.3.1] |
| 0079 | homoserine kinase (thrB) [2.7.1.39] |
| 0080 | Bifunctional aspartokinase/homoserine dehydrogenase (AK-HD)[Includes: Aspartokinase; Homoserine |
| | dehydrogenase(EC 1.1.1.3)] [2.7.2.4] |
| 0081 | conserved hypothetical protein TIGR00044 |
| 0082 | Glycerate kinase [2.7.1.31] |
| 0083 | H+/gluconate symporter (gntP) |
| 0084 | sugar diacid utilization regulator |
| 0085 | 4-hydroxybutyrate dehydrogenase (gntP) [1.1.1.61] |
| 0086 | Putative HTH-type transcriptional regulator (glpR) |
| 0087 | methyltransferase |
| 0088 | conserved hypothetical protein |
| 0089 | Iron-utilization periplasmic protein precursor (Major ferric ironbinding protein) (Iron-regulated 40 kDa protein) |
| | (MIRP) (Fe(3+)-binding protein) (hitA) |
| 0090 | hypothetical protein |
| 0091 | iron ABC transporter, permease protein hitB [validated] (III) |
| 0092 | iron utilization protein C (hitC) [3.6.3.30] |
| 0093 | D-alanyl-D-alaninecarboxypeptidase |
| 0094 | succinyl-diaminopimelate desuccinylase (dapE) [3.5.1.18] |
| 0095 | Protein [1.—.—.—] |
| 0096 | heat shock protein htpG (htpG) |
| 0097 | conserved hypothetical protein TIGR00486 |
| 0098 | signal recognition particle protein (ffh) |
| 0099 | similar to [SwissProt Accession Number P37908] |
| 0100 | Protein of unknown function (DUF1212) family |
| 0101 | Protein yjjB |
| 0102 | conserved hypothetical protein |
| 0103 | seryl-tRNA synthetase (serS) [6.1.1.11] |
| 0104 | Glutathione S-transferase (bphH) [2.5.1.18] |
| 0105 | Heme/hemopexin utilization protein C precursor (hemR) |
| 0106 | Heme/hemopexin utilization protein C precursor (hemR) |
| 0107 | predicted N6-adenine-specific DNA methylase |
| 0108 | lytic murein transglycosylase A (AF226403) [3.2.1.—] |
| 0109 | HesA/MoeB/ThiF family protein |
| 0110 | High-affinity zinc uptake system protein znuA precursor (AE005408) |
| 0111 | conserved hypothetical protein |
| 0112 | UDP-N-acetylmuramate: L-alanyl-gamma-D-glutamyl-meso-diaminopimelate ligase (mpl) |
| 0113 | cystathionine beta-lyase (metC) [4.4.1.8] |
| 0114 | TsaA (tsaA) [1.6.4.—] |
| 0115 | CDP-diacylglycerol-glycerol-3-phosphate 3-phosphatidyltransferase (pgsA) [2.7.8.5] |
| 0116 | inorganic pyrophosphatase (ppa) [3.6.1.1] |
| 0117 | xanthine/uracil permease family protein |
| 0118 | hypothetical protein |
| 0119 | uridine kinase (udk) [2.7.1.48] |
| 0120 | Deoxycytidine triphosphate deaminase (dCTP deaminase) (dcd) [3.5.4.13] |
| 0121 | conserved hypothetical protein |
| 0122 | Sugar efflux transporter |
| 0123 | GTP-binding protein engA |
| 0124 | Outer membrane protein P2 precursor (OMP P2) (ompP2) |
| 0125 | N-acetylglucosamine-6-phosphate deacetylase (nagA) [3.5.1.25] |
| 0126 | glucosamine-6-phosphate isomerase (nagB) [3.5.99.6] |
| 0127 | N-acetylneuraminate lyase (nanA) [4.1.3.3] |
| 0128 | Transcriptional regulator |
| 0129 | 2.7.1.60 [2.7.1.60] |
| 0130 | possible N-acetylmannosamine-6-P epimerase [5.1.3.9] |
| 0131 | Protein HI0146 precursor |
| 0132 | N-acetylneuraminate transporter small subunit |
| 0133 | Protein HI0148 precursor |
| 0134 | conserved hypothetical protein |
| 0135 | HflC protein (hflC) |
| 0136 | HflK protein (hflK) [3.4.—.—] |
| 0137 | Putative 4′-phosphopantetheinyl transferase [2.7.8.—] |
| 0138 | hypothetical protein |
| 0139 | anaerobic C4-dicarboxylate transporter (dcuB) |
| 0140 | Acyl carrier protein (ACP)-related protein |
| 0141 | 3-oxoacyl-[acyl-carrier protein] reductase (3-ketoacyl-acyl carrier protein reductase) (fabG) [1.1.1.100] |
| 0142 | malonyl CoA-acyl carrier protein transacylase (fabD) [2.3.1.39] |
| 0143 | hypothetical protein |
| 0144 | 3-oxoacyl-[acyl-carrier-protein] synthase III (Beta-ketoacyl-ACP synthase III) (KAS III) (fabH) [2.3.1.41] |
| 0145 | hypothetical protein |
| 0146 | ribosomal protein L32 (rpmF) |
| 0147 | Uncharacterized ACR, COG1399 |
| 0148 | phosphatidylserine decarboxylase (psd) [4.1.1.65] |
| 0149 | glutathione-disulfide reductase (gor) [1.8.1.7] |
| 0150 | Hypothetical lipoprotein HI0162 precursor |
| 0151 | BolA protein homolog (bolA) |
| 0152 | NADH: ubiquinone oxidoreductase, Na(+)-translocating, A subunit (nqrA) [1.6.5.—] |
| 0153 | NADH: ubiquinone oxidoreductase, Na(+)-translocating, B subunit (nqrB) [1.6.5.—] |
| 0154 | NADH: ubiquinone oxidoreductase, Na(+)-translocating, C subunit (nqrC) [1.6.5.—] |
| 0155 | NADH: ubiquinone oxidoreductase, Na(+)-translocating, D subunit (nqrD) [1.6.5.—] |
| 0156 | NADH: ubiquinone oxidoreductase, Na(+)-translocating, E subunit (nqrE) [1.6.5.—] |
| 0157 | NADH: ubiquinone oxidoreductase, Na(+)-translocating, F subunit (nqrF) [1.6.5.—] |
| 0158 | Thiamine biosynthesis lipoprotein apbE precursor |
| 0159 | ApbE family |
| 0160 | tRNA (5-methylaminomethyl-2-thiouridylate)-methyltransferase (trmU) [2.1.1.61] |
| 0161 | conserved hypothetical protein TIGR00726 |
| 0162 | Ribosomal large subunit pseudouridine synthase D(Pseudouridylate synthase) (Uracil hydrolyase) (sfhB) [4.2.1.70] |
| 0163 | unkown |
| 0164 | lipoprotein, putative |
| 0165 | formate acetyltransferase activating enzyme, lyase 1-specific (act) [1.97.1.4] |
| 0166 | formate acetyltransferase (pflB) [2.3.1.54] |
| 0167 | Formate transporter 1 FocA (Formate channel 1) (formate) |
| 0168 | hypothetical protein |
| 0169 | ROK family protein VC1532 |
| 0170 | sodium/alanine symporter |
| 0171 | Protein [3.1.1.1] |
| 0172 | alcohol dehydrogenase (adhC) [1.1.1.1] |
| 0173 | transcription regulator, MerR family NMB1303 (probale) |
| 0174 | o261 (YIGT) |
| 0175 | Sec-independent protein translocase protein tatB homolog |
| 0176 | Sec-independent protein translocase TatC (tatC) |
| 0177 | NADP-specific glutamate dehydrogenase (NADP-GDH) (gdhA) [1.4.1.4] |
| 0178 | iron repressor protein (fur) |
| 0179 | flavodoxin |
| 0180 | hydrolase, alpha/beta fold family (acoC) [3.1.—.—] |
| 0181 | SeqA protein |
| 0182 | O-succinylbenzoate-CoA ligase (menE) [6.2.1.26] |
| 0183 | UPF0003 protein HI0195.1 precursor (aefA) |
| 0184 | chorismate synthase (aroC) [4.2.3.5] |
| 0185 | Penicillin-insensitive murein endopeptidase precursor (mepA) [3.4.99.—] |
| 0186 | predicted permease (orf9) |
| 0187 | lipid A biosynthesis (KDO)2-(lauroyl)-lipid IVA acyltransferase (msbB) [2.3.1.—] |
| 0188 | selenide, water dikinase (selD) [2.7.9.3] |
| 0189 | ribosomal protein L19 (rplS) |
| 0190 | tRNA (guanine-N1)-methyltransferase (trmD) [2.1.1.31] |
| 0191 | 16S rRNA processing protein rimM |
| 0192 | ribosomal protein S16 (rpsP) |
| 0193 | Protein HI0205 precursor |
| 0194 | NAD pyrophosphatase/5′-nucleotidase NadN (nadN) |
| 0195 | shikimate kinase (aroK) [2.7.1.71] |
| 0196 | 3-dehydroquinate synthase (aroB) [4.2.3.4] |
| 0197 | DNA adenine methylase (Deoxyadenosyl-methyltransferase)(DNA adenine methyltransferase) (M.HindIV) (dam) |
| | [2.1.1.72] |
| 0198 | Phosphatidylglycerophosphatase B (pgpB) [3.1.3.27] |
| 0199 | GTP cyclohydrolase II (ribA) [3.5.4.25] |
| 0200 | Putative binding protein HI0213 precursor (AA1) |
| 0201 | Protein of unknown function (DUF454) family |
| 0202 | Oligopeptidase A (prlC) [3.4.24.70] |
| 0203 | type I restriction-modification system, M subunit (hsdM) [2.1.1.72] |
| 0204 | HP790-like protein (hsdS) [3.1.21.3] |
| 0205 | anticodon nuclease NMB0832 |
| 0206 | DNA-binding protein (partial) |
| 0207 | Type I site-specific deoxyribonuclease HsdR (hsdR) [3.1.21.3] |
| 0208 | membrane protein ykgB |
| 0209 | hypothetical protein |
| 0210 | aerobic respiration control sensor protein [2.7.3.—] |
| 0211 | uracil-DNA glycosylase |
| 0212 | BirA bifunctional protein [Includes: Biotin operon repressor; Biotin-[acetyl-CoA-carboxylase] synthetase(Biotin- |
| | protein ligase)] (birA) [6.3.4.15] |
| 0213 | inosine-5′-monophosphate dehydrogenase (guaB) [1.1.1.205] |
| 0214 | hypothetical protein |
| 0215 | GMP synthase [glutamine-hydrolyzing] (Glutamineamidotransferase) (GMP synthetase) (guaA) [6.3.5.2] |
| 0216 | rarD protein (rarD) |
| 0217 | AsnC-family transcriptional regulator |
| 0218 | Na+/H+ antiporter NhaA (nhaA) |
| 0219 | branched-chain amino acid transport system II carrier protein (brnQ) |
| 0220 | Glutathionylspermidine synthase (orfa) [6.3.1.8] |
| 0221 | putative cytoplasmic protein |
| 0222 | conserved hypothetical protein |
| 0223 | Protein HI0246 precursor |
| 0224 | S-adenosylmethionine: tRNA ribosyltransferase-isomerase (queA) [5.—.—.—] |
| 0225 | conserved hypothetical protein |
| 0226 | SSB (ssb) |
| 0227 | excinuclease ABC, A subunit (uvrA) |
| 0228 | 3.4.21.— (iga1) [3.4.21.—] |
| 0229 | hypothetical protein |
| 0230 | Protein |
| 0231 | conserved hypothetical protein |
| 0232 | Polyribonucleotide nucleotidyltransferase (Polynucleotidephosphorylase) (PNPase) (pnp) [2.7.7.8] |
| 0233 | Lipoprotein nlpI homolog precursor |
| 0234 | Cold-shock DEAD-box protein A homolog (ATP-dependent RNA helicase deaDhomolog) (deaD) |
| 0235 | conserved hypothetical protein |
| 0236 | Uncharacterized protein conserved in bacteria |
| 0237 | arsenate reductase (arsC) [1.20.4.1] |
| 0238 | PerM (perM) |
| 0239 | Protein-export membrane protein secF (secF) |
| 0240 | Protein-export membrane protein secD (secD) |
| 0241 | preprotein translocase, YajC subunit (yajC) |
| 0242 | Uncharacterized protein family UPF0033 superfamily |
| 0243 | Domain of Unknown function domain protein |
| 0244 | queuine tRNA-ribosyltransferase (tgt) [2.4.2.29] |
| 0245 | conserved hypothetical protein |
| 0246 | conserved hypothetical protein |
| 0247 | TonB protein (tonB) |
| 0248 | Biopolymer transport exbD protein (exbD) |
| 0249 | Biopolymer transport exbB protein (exbB) |
| 0250 | Bacterioferritin comigratory protein homolog (bcp) |
| 0251 | dihydrodipicolinate synthase (dapA) [4.2.1.52] |
| 0252 | Protein |
| 0253 | ribosomal subunit interface protein (yfiA) |
| 0254 | glycosyl transferase (glucosyl) [2.4.1.44] |
| 0255 | non-canonical purine NTP pyrophosphatase, rdgB/HAM1 family (rdgB) |
| 0256 | KDO kinase [2.7.1.—] |
| 0257 | OpsX [2.—.—.—] |
| 0258 | Heme/hemopexin utilization protein C precursor (hemR) |
| 0259 | heme-hemopexin utilization protein B (hxuB) |
| 0260 | Heme/hemopexin-binding protein precursor (Heme: hemopexin utilizationprotein A) (hxuA) |
| 0261 | heme-hemopexin utilization protein A (hxuA) |
| 0262 | dihydroneopterin aldolase (folB) [4.1.2.25] |
| 0263 | conserved hypothetical protein TIGR00023 |
| 0264 | Sensor protein narQ homolog (narQ) [2.7.3.—] |
| 0265 | UDP-N-acetylenolpyruvoylglucosamine reductase (murB) [1.1.1.158] |
| 0266 | RNA polymerase sigma-32 factor (rpoH) [2.7.7.6] |
| 0267 | tRNA-dihydrouridine synthase C [1.—.—.—] |
| 0268 | DnaJ-like protein djlA (orf81) |
| 0269 | orotate phosphoribosyltransferase (pyrE) [2.4.2.10] |
| 0270 | ribonuclease PH (rph) [2.7.7.56] |
| 0271 | glutamyl-tRNA synthetase (gltX) [6.1.1.17] |
| 0272 | conserved hypothetical protein |
| 0273 | ribonuclease BN, putative [3.1.—.—] |
| 0274 | SEC-C motif domain protein |
| 0275 | MOSC domain protein |
| 0276 | hypothetical protein |
| 0277 | uridine phosphorylase (udp) [2.4.2.3] |
| 0278 | transmembrane transport protein |
| 0279 | Predicted hydrolase or acyltransferase [3.1.—.—] |
| 0280 | 2-succinyl-6-hydroxy-2,4-cyclohexadiene-1-carboxylic acid synthase/2-oxoglutarate decarboxylase (menD) |
| | [4.1.1.71] |
| 0281 | Menaquinone-specific isochorismate synthase(Isochorismate mutase) (menF) [5.4.4.2] |
| 0282 | conserved hypothetical protein |
| 0283 | aspartate transaminase (ASPAT) [2.6.1.—] |
| 0284 | Tryptophan-specific transport protein (Tryptophan permease) (mtr) |
| 0285 | L-serine ammonia-lyase (sdaA) [4.3.1.17] |
| 0286 | Serine transporter (sdaC) |
| 0287 | copper-translocating P-type ATPase [3.6.3.4] |
| 0288 | periplasmic mercuric ion binding protein (merP) |
| 0289 | heavy-metal transporting P-type ATPase CAC3655 (merP) |
| 0290 | heavy-metal transporting P-type ATPase CAC3655 (merP) |
| 0291 | heavy-metal transporting CPx-type ATPase (merP) |
| 0292 | Cu(I)-responsive transcriptional regulator (cueR) |
| 0293 | MetApo-repressor, MetJ |
| 0294 | transcription termination factor Rho (rho) |
| 0295 | PilD (hopD) [3.4.23.43] |
| 0296 | Protein transport protein hofC homolog (pilC) |
| 0297 | Protein transport protein hofB homolog (pilB) |
| 0298 | Prepilin peptidase dependent protein D homolog precursor (PilE) |
| 0299 | AmpD protein homolog (ampD) |
| 0300 | Magnesium and cobalt efflux protein corC (tlyC) |
| 0301 | Apolipoprotein N-acyltransferase (ALP N-acyltransferase)(Copper homeostasis protein cutE homolog) (cutE) |
| | [2.3.1.—] |
| 0302 | conserved hypothetical protein TIGR00046 |
| 0303 | Uncharacterized ACR, COG1678 |
| 0304 | conserved hypothetical protein TIGR00250 |
| 0305 | Recombination associated protein rdgC |
| 0306 | pyrroline-5-carboxylate reductase (proC) [1.5.1.2] |
| 0307 | MFS transporter |
| 0308 | tyrosine recombinase XerD (xerD) |
| 0309 | conserved hypothetical protein |
| 0310 | Holliday junction DNA helicase RuvB (ruvB) |
| 0311 | Holliday junction DNA helicase RuvA (ruvA) |
| 0312 | crossover junction endodeoxyribonuclease RuvC (ruvC) [3.1.22.4] |
| 0313 | conserved hypothetical protein TIGR01033 |
| 0314 | dATP pyrophosphohydrolase (ntpA) [3.6.1.—] |
| 0315 | aspartyl-tRNA synthetase (aspS) [6.1.1.12] |
| 0316 | HI0318 homolog |
| 0317 | methyltransferase, putative |
| 0318 | lactoylglutathione lyase (gloA) [4.4.1.5] |
| 0319 | ribonuclease T (rnt) [3.1.13.—] |
| 0320 | predicted permease |
| 0321 | conserved hypothetical protein |
| 0322 | translation elongation factor P (efp) |
| 0323 | lysine 2; 3-aminomutase |
| 0324 | Opacity associated proteins oapA (oapA) |
| 0325 | OapB (oapB) |
| 0326 | DNA repair protein RecO (recO) |
| 0327 | 23S rRNA (uracil-5-)-methyltransferase RumA (rumA) [2.1.1.—] |
| 0328 | GTP pyrophosphokinase (ATP: GTP 3′-pyrophosphotransferase)(ppGpp synthetase I) ((P)ppGpp synthetase) (relA) |
| | [2.7.6.5] |
| 0329 | Diacylglycerol kinase (DAGK) (Diglyceride kinase)(DGK) (dgkA) [2.7.1.107] |
| 0330 | Molybdopterin biosynthesis mog protein (mog) |
| 0331 | Nitrogen regulatory protein P-II |
| 0332 | Domain of unknown function, putative |
| 0333 | Primosomal protein N′ (Replication factor Y) (priA) |
| 0334 | tRNA (guanine-N(7)-)-methyltransferase (tRNA(m7G46)-methyltransferase) [2.1.1.33] |
| 0335 | Protein |
| 0336 | ferredoxin-type protein NapF (napF) |
| 0337 | NapD protein (napD) |
| 0338 | periplasmic nitrate reductase, large subunit (napA) [1.7.99.4] |
| 0339 | Ferredoxin-type protein napG homolog (napG) |
| 0340 | Ferredoxin-type protein napH homolog (napH) |
| 0341 | periplasmic nitrate reductase, diheme cytochrome c subunit (napB) |
| 0342 | Cytochrome c-type protein napC (napC) |
| 0343 | adenylate kinase (adk) [2.7.4.3] |
| 0344 | unnamed protein product; ORF3 (ampG1) |
| 0345 | UDP-glucose 4-epimerase (galE) [5.1.3.2] |
| 0346 | unnamed protein product; ORF1 |
| 0347 | ABC transporter, ATP-binding protein |
| 0348 | ABC transporter permease protein (permease) |
| 0349 | thiamin biosynthesis associated protein (nmt1) |
| 0350 | transcription activator tenA (paralogs) |
| 0351 | YfeD (chelated) |
| 0352 | YfeC (chelated) |
| 0353 | YfeB (chelated) |
| 0354 | YfeA (chelated) |
| 0355 | hypothetical protein |
| 0356 | Penicillin-binding protein 7 homolog precursor (PBP-7) (D-alanyl-D-alanine-endopeptidase) (DD-endopeptidase) |
| | [3.4.99.—] |
| 0357 | hypothetical protein |
| 0358 | radical SAM enzyme, Cfr family |
| 0359 | possible fimbrial biogenesis and twitching motility protein PilF homolog (Tfp) |
| 0360 | conserved hypothetical protein |
| 0361 | 4-hydroxy-3-methylbut-2-en-1-yl diphosphate synthase (ispG) [1.17.4.3] |
| 0362 | histidyl-tRNA synthetase (hisS) [6.1.1.21] |
| 0363 | Protein |
| 0364 | Protein of unknown function (DUF528) superfamily |
| 0365 | ferredoxin, 2Fe—2S type, ISC system (fdx) |
| 0366 | Fe—S protein assembly chaperone HscA (hscA) |
| 0367 | conserved hypothetical protein |
| 0368 | co-chaperone Hsc20 (hscB) |
| 0369 | iron-sulfur cluster assembly protein IscA (iscA) |
| 0370 | FeS cluster assembly scaffold IscU (iscU) |
| 0371 | cysteine desulfurase IscS (iscS) [4.4.1.—] |
| 0372 | iron-sulfur cluster assembly transcription factor IscR (iscR) |
| 0373 | RNA methyltransferase, TrmH family, group 1 |
| 0374 | Outer membrane protein P6 precursor (OMP P6) (15 kDa peptidoglycan-associated lipoprotein) (PC protein) (pal) |
| 0375 | TolB protein precursor (tolB) |
| 0376 | TolA protein (tolA) |
| 0377 | TolR protein (tolR) |
| 0378 | TolQ protein (tolQ) |
| 0379 | YbgC protein |
| 0380 | Dnt (dinG) |
| 0381 | Inactive homolog of metal-dependent proteases (M22) [3.4.—.—] |
| 0382 | starvation-inducible outer membrane lipoprotein |
| 0383 | Long-chain-fatty-acid-CoA ligase (Long-chain acyl-CoAsynthetase) (fadD) [6.2.1.3] |
| 0384 | ribonuclease D (rnd) [3.1.26.3] |
| 0385 | O-antigen acetylase XF0778 (LPS) |
| 0386 | GTP-binding protein YchF (ychF) |
| 0387 | peptidyl-tRNA hydrolase (pth) [3.1.1.29] |
| 0388 | Protein |
| 0389 | Protein |
| 0390 | exodeoxyribonuclease VII, large subunit (xseA) [3.1.11.6] |
| 0391 | conserved hypothetical protein TIGR00052 |
| 0392 | Icc protein homolog (icc) [3.1.4.17] |
| 0393 | C4-dicarboxylate transport protein homolog b2343 |
| 0394 | Outer membrane protein P1 precursor (OMP P1) (fadL) |
| 0395 | Methylated-DNA-protein-cysteine methyltransferase (6-O-methylguanine-DNA methyltransferase) (O-6- |
| | methylguanine-DNA-alkyltransferase) (dat1) [2.1.1.63] |
| 0396 | DNA mismatch repair protein mutH (mutH) |
| 0397 | MesJ (mesJ) |
| 0398 | pyridoxal kinase [2.7.1.35] |
| 0399 | acetyl-CoA carboxylase, carboxyl transferase, alpha subunit (accA) [6.4.1.2] |
| 0400 | High-affinity zinc uptake system membrane protein znuB (PA5501) |
| 0401 | High-affinity zinc uptake system ATP-binding protein znuC (afuC) |
| 0402 | hypothetical protein |
| 0403 | membrane protein ECs2566 [similarity] [3.4.24.—] |
| 0404 | Transcriptional regulatory protein tyrR homolog (tyrR) |
| 0405 | host factor I (hfq) |
| 0406 | Ribosomal large subunit pseudouridine synthase C(Pseudouridylate synthase) (Uracil hydrolyase) (orfx) [4.2.1.70] |
| 0407 | ribonuclease E (rne) [3.1.4.—] |
| 0408 | Opa protein |
| 0409 | hydroxyethylthiazole kinase (thiM) [2.7.1.50] |
| 0410 | phosphomethylpyrimidine kinase (thiD) [2.7.4.7] |
| 0411 | thiamine-phosphate pyrophosphorylase (thiE) [2.5.1.3] |
| 0412 | major facilitator family transporter (AE005578) |
| 0413 | hypothetical protein |
| 0414 | collagenase (prtC) [3.4.—.—] |
| 0415 | ATP-dependent RNA helicase srmB homolog (srmB) |
| 0416 | Predicted O-methyltransferase (putative) |
| 0417 | YfiF protein (Fragment) [2.1.1.—] |
| 0418 | CDP-diacylglycerol-serine O-phosphatidyltransferase(Phosphatidylserine synthase) (pssA) [2.7.8.8] |
| 0419 | Fatty acid metabolism regulator protein (fadR) |
| 0420 | Na+/H+ antiporter NhaB (nhaB) |
| 0421 | Disulfide bond formation protein B (Disulfide oxidoreductase) (dsbB) [1.8.4.—] |
| 0422 | glucosamine--fructose-6-phosphate aminotransferase, isomerizing (glmS) [2.6.1.16] |
| 0423 | DNA-binding protein HU-2 (hupA) |
| 0424 | histidine-tRNA ligase (hisS) [6.1.1.21] |
| 0425 | Protein |
| 0426 | starvation-inducible outer membrane lipoprotein |
| 0427 | Long-chain-fatty-acid--CoA ligase (Long-chain acyl-CoAsynthetase) (fadD) [6.2.1.3] |
| 0428 | Protein |
| 0429 | NADH pyrophosphatase (MutT) [3.6.1.—] |
| 0430 | Protein HI0433 (ORFG) |
| 0431 | Competence protein F (DNA transformation protein comF) (ProteinCOM101A) (comF) |
| 0432 | Competence protein E precursor (DNA transformation protein comE) (comE) |
| 0433 | hypothetical protein |
| 0434 | Competence protein D (DNA transformation protein comD) (comD) |
| 0435 | Competence protein C (DNA transformation protein comC) (comC) |
| 0436 | Competence protein B (DNA transformation protein comB) (comB) |
| 0437 | Competence protein A (DNA transformation protein comA) (comA) |
| 0438 | penicillin-binding protein 1A (ponA) [2.4.2.—] |
| 0439 | Protein HI0441 (ORFJ) |
| 0440 | hypothetical protein |
| 0441 | conserved hypothetical protein TIGR00103 |
| 0442 | recombination protein RecR (recR) |
| 0443 | DNA topoisomerase III (topB) [5.99.1.2] |
| 0444 | Protein-export membrane protein secG (secG) |
| 0445 | CP4-57 integrase-like protein |
| 0446 | phage phi-R73 primase-like protein [2.7.7.—] |
| 0447 | conserved hypothetical protein |
| 0448 | conserved hypothetical protein |
| 0449 | hypothetical protein |
| 0450 | hypothetical protein |
| 0451 | conserved hypothetical protein |
| 0452 | conserved hypothetical protein |
| 0453 | conserved hypothetical protein |
| 0454 | phage-related protein |
| 0455 | conserved hypothetical protein |
| 0456 | hypothetical protein |
| 0457 | conserved hypothetical protein |
| 0458 | phage terminase, large subunit, putative |
| 0459 | phage terminase, small subunit, putative, P27 family |
| 0460 | phage phi-105 holin-like protein |
| 0461 | Phage QLRG family, putative DNA packaging |
| 0462 | phage head-tail adaptor, putative |
| 0463 | phage portal protein, HK97 family |
| 0464 | Caudovirus prohead protease |
| 0465 | phage major capsid protein, HK97 family |
| 0466 | PTS system, fructose-specific IIBC component (EIIBC-Fru) (Fructose-permease IIBC component) |
| | (Phosphotransferase enzyme II, BC component)(EG 2.7.1.69) (EII-Fru) (fruA) [2.7.1.69] |
| 0467 | 1-phosphofructokinase (Fructose 1-phosphate kinase) (fruK) [2.7.1.56] |
| 0468 | PTS system, fructose-specific IIA/FPr component (EIIA-Fru) (Fructose-permease IIA/FPr component) |
| | (Phosphotransferase enzyme II, A/FPrcomponent) (Phosphotransferase FPr protein) (Pseudo-HPr) (EIII-Fru) |
| | (Fructose PTS diphosphoryl transfer protein) (P17127) [2.7.1.69] |
| 0469 | lipoprotein, putative |
| 0470 | Virulence-associated protein D (vapD) |
| 0471 | Virulence-associated protein D (vapD) |
| 0472 | conserved hypothetical protein |
| 0473 | CBS domain protein (AF212041) |
| 0474 | Protein of unknown function (DUF1523) superfamily |
| 0475 | HI0454 [3.1.21.—] |
| 0476 | DNA polymerase III, delta′ subunit (holB) [2.7.7.7] |
| 0477 | thymidylate kinase (tmk) [2.7.4.9] |
| 0478 | conserved hypothetical protein TIGR00247 |
| 0479 | conserved hypothetical protein TIGR00247 |
| 0480 | Survival protein surA homolog precursor (PPIase) [5.2.1.8] |
| 0481 | PyrR bifunctional protein [Includes: Pyrimidine operon regulatoryprotein; Uracil phosphoribosyltransferase |
| | (UPRTase)] (pyrR) [2.4.2.9] |
| 0482 | MazG protein homolog (mazG) |
| 0483 | Protein (lapB) |
| 0484 | ATP-dependent protease La (lon) [3.4.21.53] |
| 0485 | oxygen-independent coproporphyrinogen III oxidase, putative |
| 0486 | ribose 5-phosphate isomerase A (rpiA) [5.3.1.6] |
| 0487 | D-3-phosphoglycerate dehydrogenase (PGDH) (serA) [1.1.1.95] |
| 0488 | Predicted aminomethyltransferase |
| 0489 | conserved hypothetical protein TIGR00255 |
| 0490 | ATP phosphoribosyltransferase (hisG) [2.4.2.17] |
| 0491 | hypothetical protein |
| 0492 | ATP phosphoribosyltransferase (hisG) [2.4.2.17] |
| 0493 | histidinol dehydrogenase (hisD) [1.1.1.23] |
| 0494 | histidinol-phosphate aminotransferase (hisC) [2.6.1.9] |
| 0495 | Histidine biosynthesis bifunctional protein hisB [Includes: Histidinol-phosphatase; Imidazoleglycerol- |
| | phosphatedehydratase (EC 4.2.1.19) (IGPD)] [3.1.3.15] |
| 0496 | imidazole glycerol phosphate synthase, glutamine amidotransferase subunit (hisH) [2.4.2.—] |
| 0497 | phosphoribosylformimino-5-aminoimidazole carboxamide ribotide isomerase (hisA) [5.3.1.16] |
| 0498 | imidazoleglycerol phosphate synthase, cyclase subunit (hisF) |
| 0499 | Histidine biosynthesis bifunctional protein hislE [Includes: Phosphoribosyl-AMP cyclohydrolase (PRA- |
| | CH); Phosphoribosyl-ATP pyrophosphatase (EC 3.6.1.31) (PRA-PH)] (PRA-CH) [3.5.4.19] |
| 0500 | Tyrosine-specific transport protein 1 (Tyrosine permease 1) (tyrP) |
| 0501 | ATP synthase F1, epsilon subunit (atpC) [3.6.3.14] |
| 0502 | ATP synthase F1, beta subunit (atpD) [3.6.3.14] |
| 0503 | ATP synthase F1, gamma subunit (atpG) [3.6.3.14] |
| 0504 | ATP synthase F1, alpha subunit (atpA) [3.6.3.14] |
| 0505 | ATP synthase F1, delta subunit (atpH) [3.6.3.14] |
| 0506 | ATP synthase F0, B subunit (atpF) [3.6.3.14] |
| 0507 | ATP synthase C chain (Lipid-binding protein)(Dicyclohexylcarbodiimide-binding protein) (atpE) [3.6.3.14] |
| 0508 | ATP synthase F0, A subunit (atpB) [3.6.3.14] |
| 0509 | conserved hypothetical protein |
| 0510 | methyltransferase GidB (gidB) |
| 0511 | conserved hypothetical protein |
| 0512 | CbbY family protein VCA0662 |
| 0513 | predicted membrane protein |
| 0514 | autoinducer-2 production protein LuxS (luxS) |
| 0515 | transposase |
| 0516 | HAD superfamily (subfamily IIIB) phosphatase, TIGR01672 (AphA) [3.1.3.—] |
| 0517 | heat shock protein (hslV) [3.4.25.—] |
| 0518 | heat shock protein HslVU, ATPase subunit HslU (hslU) |
| 0519 | spermidine/putrescine-binding protein 2 precursor (potD) |
| 0520 | OrdL [1.—.—.—] |
| 0521 | DNA recombination protein rmuC homolog (YIGN) |
| 0522 | High affinity ribose transport protein rbsD (rbsD) |
| 0523 | Ribose transport ATP-binding protein rbsA (rbsA) |
| 0524 | Ribose transport system permease protein rbsC (rbsC) |
| 0525 | D-ribose-binding periplasmic protein precursor (rbsB) |
| 0526 | ribokinase (rbsK) [2.7.1.15] |
| 0527 | rbs repressor homolog (rbsR) |
| 0528 | conserved hypothetical protein TIGR00645 |
| 0529 | protein of unknown function, TIGR01935 |
| 0530 | 1,4-dihydroxy-2-naphthoate octaprenyltransferase (menA) [2.5.—.—] |
| 0531 | Protein rcsF (orf3) |
| 0532 | Tellurite resistance protein tehA homolog (tehA) |
| 0533 | DNA-directed RNA polymerase beta′ chain (RNAP beta′subunit) (Transcriptase beta′ chain) (RNA polymerase beta′ |
| | subunit) (rpoC) [2.7.7.6] |
| 0534 | DNA-directed RNA polymerase, beta subunit (rpoB) [2.7.7.6] |
| 0535 | ribosomal protein L1 (rplA) |
| 0536 | ribosomal protein L11 (rplK) |
| 0537 | purine nucleoside phosphorylase (deoD) [2.4.2.1] |
| 0538 | NupC family protein VC2352 |
| 0539 | NupC family protein VC2352 (nupC) |
| 0540 | Pyruvate-formate lyase-activating enzyme |
| 0541 | Protein |
| 0542 | ADP-heptose--lipooligosaccharide heptosyltransferase III (RFAF) |
| 0543 | Uncharacterized BCR, YitT family COG1284 subfamily, putative |
| 0544 | fructose-bisphosphate aldolase, class II (fbaA) [4.1.2.13] |
| 0545 | phosphoglycerate kinase (pgk) [2.7.2.3] |
| 0546 | unnamed protein product; Some similarities with ribonuclease |
| 0547 | ferredoxin (fdx) |
| 0548 | Tyrosine-specific transport protein 2 (Tyrosine permease 2) (tyrP) |
| 0549 | thymidine kinase |
| 0550 | Gcp (gcp) [3.4.24.57] |
| 0551 | ribosomal protein S21 (rpsU) |
| 0552 | DNA primase (dnaG) [2.7.7.—] |
| 0553 | RNA polymerase sigma factor rpoD (Sigma-70) (rpoD) |
| 0554 | aspartate ammonia-lyase (aspA) [4.3.1.1] |
| 0555 | Urease accessory protein ureH (ureH) |
| 0556 | urease accessory protein UreG (ureG) |
| 0557 | Urease accessory protein ureF (ureF) |
| 0558 | Urease accessory protein ureE (ureE) |
| 0559 | urease, alpha subunit (ureC) [3.5.1.5] |
| 0560 | urease, beta subunit (ureB) [3.5.1.5] |
| 0561 | urease, gamma subunit (ureA) [3.5.1.5] |
| 0562 | chaperonin, 10 kDa (groES) |
| 0563 | 60 kDa chaperonin (Protein Cpn60) (groEL protein) (groEL) |
| 0564 | ribosomal protein L9 (rplI) |
| 0565 | ribosomal protein S18 (rpsR) |
| 0566 | Single-strand binding protein family |
| 0567 | 30S ribosomal protein (rpS6) |
| 0568 | translation initiation factor IF-1 (infA) |
| 0569 | Lipooligosaccharide biosynthesis protein lic2B [2.—.—.—] |
| 0570 | LgtG |
| 0571 | dimethyladenosine transferase (ksgA) [2.1.1.—] |
| 0572 | lipopolysaccharide core [2.—.—.—] |
| 0573 | bis(5′-nucleosyl)-tetraphosphatase (symmetrical) [3.6.1.41] |
| 0574 | conserved hypothetical protein |
| 0575 | 6-phosphogluconate dehydrogenase, decarboxylating (gnd) [1.1.1.44] |
| 0576 | conserved hypothetical protein |
| 0577 | hypothetical protein |
| 0578 | integral membrane protein |
| 0579 | 6-phosphogluconolactonase (pgl) [3.1.1.31] |
| 0580 | glucose-6-phosphate 1-dehydrogenase (zwf) [1.1.1.49] |
| 0581 | 3′(2′),5′-bisphosphate nucleotidase (cysQ) [3.1.3.7] |
| 0582 | conserved hypothetical protein |
| 0583 | oligopeptide transporter, OPT family |
| 0584 | Heat shock protein 15 homolog (HSP15) (HSP15) |
| 0585 | Regulatory protein asnC (asnC) |
| 0586 | aspartate-ammonia ligase (asnA) [6.3.1.1] |
| 0587 | phosphoglycolate phosphatase, bacterial (gph) [3.13.18] |
| 0588 | ribulose-phosphate 3-epimerase (rpe) [5.1.3.1] |
| 0589 | DNA gyrase, B subunit (gyrB) [5.99.1.3] |
| 0590 | hypothetical protein |
| 0591 | transcription accessory protein tex homolog (tex) |
| 0592 | transcription elongation factor GreB (greB) |
| 0593 | possible tetR family transcriptional regulator |
| 0594 | Hydrogen peroxide-inducible genes activator (oxyR) |
| 0595 | Protein [1.11.1.—] |
| 0596 | Protein slyX homolog-related protein |
| 0597 | SlyD (fkpA) [5.2.1.8] |
| 0598 | Uncharacterized protein conserved in bacteria |
| 0599 | Intracellular sulfur oxidation protein dsrE |
| 0600 | DsrF family protein |
| 0601 | conserved hypothetical protein |
| 0602 | translation elongation factor Tu (tuf) |
| 0603 | translation elongation factor G (fusA) |
| 0604 | ribosomal protein S7 (rpsG) |
| 0605 | ribosomal protein S12 (rpsL) |
| 0606 | glucose-inHIBited division protein A (gidA) |
| 0607 | 2′,3′-cyclic-nucleotide 2′-phosphodiesterase (cpdB) [3.1.4.16] |
| 0608 | Aminobenzoyl-glutamate utilization protein A homolog [3.5.—.—] |
| 0609 | c4-dicarboxylate anaerobic carrier family protein subfamily |
| 0610 | peptidase E homolog (pepE) [3.4.13.21] |
| 0611 | Positive regulator of sigma(E), RseC/MucC superfamily |
| 0612 | Putrescine-ornithine antiporter (Putrescine transport protein) (potE) |
| 0613 | Ornithine decarboxylase (speF) [4.1.1.17] |
| 0614 | transcription regulator azlB (lrp) |
| 0615 | Transporter |
| 0616 | carbamate kinase (arcC) [2.7.2.2] |
| 0617 | ornithine carbamoyltransferase (argF) [2.1.3.3] |
| 0618 | predicted hydrolase (YIGL) |
| 0619 | crcB protein (crcB) |
| 0620 | regulatory protein RecX (recX) |
| 0621 | recA protein (recA) |
| 0622 | TfoX (tfoX) |
| 0623 | translation elongation factor Tu (tuf) |
| 0624 | PsiE protein homolog |
| 0625 | hemY protein (hemY) |
| 0626 | Protein (hemX) [2.1.1.107] |
| 0627 | adenylate cyclase, class-I (cyaA) [4.6.1.1] |
| 0628 | Glycerol-3-phosphate dehydrogenase [NAD(P)+] (NAD(P)H-dependent glycerol-3-phosphate dehydrogenase) |
| | (gpsA) [1.1.1.94] |
| 0629 | Serine acetyltransferase (SAT) (cysE) [2.3.1.30] |
| 0630 | shikimate 5-dehydrogenase/quinate 5-dehydrogenase family protein (aroe) [1.1.1.25] |
| 0631 | unnamed protein product; Similar to sodium: sulfate symporter-family protein (huNaDC) |
| 0632 | methylenetetrahydrofolate dehydrogenase (NADP)/methenyltetrahydrofolate cyclohydrolase (EC |
| | 3.5.4.9) (folD) [1.5.1.5] |
| 0633 | L-fucose permease (fucP) |
| 0634 | L-fuculose phosphate aldolase (fucA) [4.1.2.17] |
| 0635 | Fucose operon fucU protein (fucU) |
| 0636 | L-fuculokinase (L-fuculose kinase) (fucK) [2.7.1.51] |
| 0637 | L-fucose isomerase (fucI) [5.3.1.25] |
| 0638 | L-fucose operon activator (fucR) |
| 0639 | RNA polymerase associated protein rapA (ATP-dependenthelicase hepA) (hepA) [3.6.1.—] |
| 0640 | Ribosomal large subunit pseudouridine synthase A(Pseudouridylate synthase) (Uracil hydrolyase) [4.2.1.70] |
| 0641 | Protein glpG homolog [3.4.21.—] |
| 0642 | Glycerol-3-phosphate regulon repressor (glpR) |
| 0643 | 28 3 kDa membrane protein (hlpA) |
| 0644 | D-methionine transport system permease protein MetI (membrane) |
| 0645 | D-methionine transport ATP-binding protein MetN (atp_bind) |
| 0646 | histidinol phosphatase |
| 0647 | peptide deformylase (def) [3.5.1.88] |
| 0648 | methionyl-tRNA formyltransferase (fmt) [2.1.2.9] |
| 0649 | sun protein (sun) [2.1.1.—] |
| 0650 | Trk system potassium uptake protein trkA (K(+)-uptake protein trkA) (trkA) |
| 0651 | large conductance mechanosensitive channel protein (mscL) |
| 0652 | Uncharacterized conserved protein |
| 0653 | RNA polymerase sigma-E factor (Sigma-24) (rpoE) |
| 0654 | Sigma-E factor negative regulatory protein homolog (mclA) |
| 0655 | Sigma-E factor regulatory protein rseB homolog precursor (rseB) |
| 0656 | pantothenate kinase (coaA) [2.7.1.33] |
| 0657 | translation elongation factor EF-Tu (tufB) |
| 0658 | translation elongation factor EF-Tu (tufB) |
| 0659 | translation elongation factor EF-Tu (tufB) |
| 0660 | conserved hypothetical protein |
| 0661 | hypothetical protein |
| 0662 | conserved hypothetical protein |
| 0663 | tRNA-dihydrouridine synthase A [1.—.—.—] |
| 0664 | C4-dicarboxylate transport protein homolog b2343 |
| 0665 | tryptophanyl-tRNA synthetase (trpS) [6.1.1.2] |
| 0666 | uncharacterized protein conserved in bacteria |
| 0667 | adenylosuccinate lyase (purB) [4.3.2.2] |
| 0668 | ribosomal protein (rpL10) |
| 0669 | ribosomal protein L7/L12 (rplL) |
| 0670 | UDP-N-acetylglucosamine pyrophosphorylase (glmU) [2.7.7.23] |
| 0671 | conserved hypothetical protein |
| 0672 | PldB (pldB) [3.1.1.5] |
| 0673 | aspartate-semialdehyde dehydrogenase (asd) [1.2.1.11] |
| 0674 | Membrane protein, MgtC/SapB family |
| 0675 | Fe—S oxidoreductase [1.8.—.—] |
| 0676 | drug activity modulator B (mdaB) [1.6.99.—] |
| 0677 | ATP-dependent DNA helicase Rep (rep) [3.6.1.—] |
| 0678 | Protein of unknown function (DUF1375) superfamily |
| 0679 | pantetheine-phosphate adenylyltransferase (coaD) [2.7.7.3] |
| 0680 | 3-deoxy-D-manno-octulosonic-acid transferase (KDOtransferase) (kdtA) [2.—.—.—] |
| 0681 | UDP-glucose-Lipooligosaccharide beta 1-4 glucosyltransferase [2.—.—.—] |
| 0682 | DNA-3-methyladenine glycosylase (3-methyladenine-DNAglycosidase) (TAG) (tagI) [3.2.2.20] |
| 0683 | hypothetical protein |
| 0684 | shikimate 5-dehydrogenase (aroE) [1.1.1.25] |
| 0685 | Protein (SUA5) |
| 0686 | DNA topoisomerase (topA) [5.99.1.2] |
| 0687 | ATPase components of ABC transporters with duplicated ATPase domains |
| 0688 | Predicted transcriptional regulators |
| 0689 | conserved hypothetical protein |
| 0690 | Hemoglobin and hemoglobin-haptoglobin binding protein B precursor |
| 0691 | CydD (cydD) |
| 0692 | ABC transporter, ATP-binding/permease protein |
| 0693 | conserved hypothetical protein |
| 0694 | conserved hypothetical protein |
| 0695 | conserved hypothetical protein |
| 0696 | Putative HTH-type transcriptional regulator HI0666.1 |
| 0697 | fructose-1,6-bisphosphatase, class II (glpX) [3.1.3.11] |
| 0698 | uncharacterized protein conserved in bacteria |
| 0699 | Protein mioC homolog (mioC) [1.8.1.2] |
| 0700 | D-tyrosyl-tRNA(Tyr) deacylase (dtd) [3.1.—.—] |
| 0701 | 2C-methyl-D-erythritol 2,4-cyclodiphosphate synthase (ispF) [4.6.1.12] |
| 0702 | 2-C-methyl-D-erythritol 4-phosphate cytidylyltransferase (ispD) [2.7.7.60] |
| 0703 | Cell division protein ftsB homolog |
| 0704 | Xanthine-guanine phosphoribosyltransferase (XGPRT) (gptB) [2.4.2.22] |
| 0705 | X-His dipeptidase (pepD) [3.4.13.3] |
| 0706 | tyrosine recombinase XerC (xerC) |
| 0707 | acetyltransferase, GNAT family |
| 0708 | triosephosphate isomerase (tpiA) [5.3.1.1] |
| 0709 | Thiosulfate sulfurtransferase glpE (glpE) [2.8.1.1] |
| 0710 | rarD protein (rarD) |
| 0711 | HTH-type transcriptional activator ilvY (ilvY) |
| 0712 | hypothetical protein |
| 0713 | ketol-acid reductoisomerase (ilvC) [1.1.1.86] |
| 0714 | Anaerobic glycerol-3-phosphate dehydrogenase subunit C (G-3-Pdehydrogenase) (glpC) [1.1.99.5] |
| 0715 | Anaerobic glycerol-3-phosphate dehydrogenase subunit B(Anaerobic G-3-P dehydrogenase subunit B) (Anaerobic |
| | G3Pdhase B) (glpB) [1.1.99.5] |
| 0716 | Anaerobic glycerol-3-phosphate dehydrogenase subunit A(G-3-P dehydrogenase) (glpA) [1.1.99.5] |
| 0717 | glycerol-3-phosphate transporter (glpT) |
| 0718 | glycerophosphodiester phosphodiesterase precursor (glpQ) [3.1.4.46] |
| 0719 | Glycerol uptake facilitator protein (glpF) |
| 0720 | glycerol kinase (glpK) [2.7.1.30] |
| 0721 | Xanthine-guanine phosphoribosyltransferase (XGPRT) (gptB) [2.4.2.22] |
| 0722 | glycerophosphodiester phosphodiesterase (glpQ) [3.1.4.46] |
| 0723 | 5′-nucleotidase, lipoprotein e(P4) family |
| 0724 | Ribosomal large subunit pseudouridine synthase E(Pseudouridylate synthase) (Uracil hydrolyase) [4.2.1.70] |
| 0725 | exopolyphosphatase [3.6.1.11] |
| 0726 | Protein |
| 0727 | Protein HI0698 precursor |
| 0728 | FKBP-type peptidyl-prolyl cis-trans isomerase slyD(PPiase) (Rotamase) (slyD) [5.2.1.8] |
| 0729 | Protein |
| 0730 | conserved hypothetical protein TIGR00094 |
| 0731 | acid phosphatase SurE (surE) [3.1.3.2] |
| 0732 | LppB (lppB) |
| 0733 | Outer membrane antigenic lipoprotein B precursor |
| 0734 | Outer membrane antigenic lipoprotein B precursor |
| 0735 | Tryptophanase (L-tryptophan indole-lyase) (TNase) (TNase) [4.1.99.1] |
| 0736 | TnaB (mtr) |
| 0737 | DNA mismatch repair protein MutS (mutS) |
| 0738 | L-seryl-tRNA selenium transferase (selA) [2.9.1.1] |
| 0739 | selenocysteine-specific translation elongation factor (selB) |
| 0740 | lipoprotein, putative |
| 0741 | negative regulator of translation |
| 0742 | conserved hypothetical protein TIGR00053 |
| 0743 | Hemoglobin and hemoglobin-haptoglobin binding protein C precursor |
| 0744 | PT repeat family |
| 0745 | trigger factor (tig) [5.2.1.8] |
| 0746 | ATP-dependent Clp protease, proteolytic subunit ClpP (clpP) [3.4.21.92] |
| 0747 | ATP-dependent Clp protease, ATP-binding subunit ClpX (clpX) |
| 0748 | Preprotein translocase secE subunit (secE) |
| 0749 | transcription termination/antitermination factor NusG (nusG) |
| 0750 | VacJ lipoprotein homolog precursor (vacJ) |
| 0751 | endoribonuclease L-PSP, putative |
| 0752 | hypothetical protein |
| 0753 | protease HtpX (heat shock protein) (htpX) [3.4.24.—] |
| 0754 | Predicted redox protein, regulator of disulfide bond formation |
| 0755 | conserved hypothetical protein TIGR00257 |
| 0756 | Trk system potassium uptake protein trkH (trkH) |
| 0757 | Hypothetical UPF0241 protein |
| 0758 | conserved hypothetical protein |
| 0759 | Nitrate/nitrite response regulator protein homolog (narP) |
| 0760 | diaminopimelate decarboxylase (lysA) [4.1.1.20] |
| 0761 | lipoprotein, putative |
| 0762 | CyaY protein (cyaY) |
| 0763 | ATP-dependent DNA helicase RecQ (recQ) [3.6.1.—] |
| 0764 | prolyl-tRNA synthetase (proS) [6.1.1.15] |
| 0765 | Organic solvent tolerance protein precursor |
| 0766 | Protein sufI homolog precursor (sufI) |
| 0767 | 1-acyl-sn-glycerol-3-phosphate acyltransferase (1-AGPacyltransferase) (1-AGPAT) (Lysophosphatidic acid |
| | acyltransferase)(LPAAT) (plsC) [2.3.1.51] |
| 0768 | UDP-2,3-diacylglucosamine hydrolase (lpxH) [3.6.1.—] |
| 0769 | sodium- and chloride-dependent transporter NMB1975 (SNF) |
| 0770 | IlvG (ALS-II) [4.1.3.18] |
| 0771 | Na+-dependent transporters of the SNF family (SNF) |
| 0772 | IlvG (ILVG) [4.1.3.18] |
| 0773 | dihydroxy-acid dehydratase (ilvD) [4.2.1.9] |
| 0774 | threonine ammonia-lyase, biosynthetic (ilvA) [4.3.1.19] |
| 0775 | DNA polymerase III alpha subunit (dnaE) [2.7.7.7] |
| 0776 | hypothetical protein |
| 0777 | YhxB (cpsG) [5.4.2.8] |
| 0778 | protein-export protein SecB (secB) |
| 0779 | rhodanese domain protein |
| 0780 | L-asparaginase II (ansB) [3.5.1.1] |
| 0781 | L-asparaginase II (ansB) [3.5.1.1] |
| 0782 | Anaerobic C4-dicarboxylate transporter dcuB (dcuB) |
| 0783 | NADH dehydrogenase (ndh) [1.6.99.3] |
| 0784 | Glycerol-3-phosphate acyltransferase (GPAT) (plsB) [2.3.1.15] |
| 0785 | LexA repressor (lexA) [3.4.21.88] |
| 0786 | diaminopimelate epimerase (dapF) [5.1.1.7] |
| 0787 | thiol peroxidase (tpx) [1.11.1.—] |
| 0788 | phosphoribosylformylglycinamidine synthase (purL) [6.3.5.3] |
| 0789 | Lex2A |
| 0790 | Lex2B (lex2B) [2.—.—.—] |
| 0791 | yibQ gene product |
| 0792 | M23/M37 peptidase domain protein protein |
| 0793 | 2,3-bisphosphoglycerate-dependent phosphoglycerate mutase(Phosphoglyceromutase) (PGAM) (BPG-dependent |
| | PGAM) (dPGM) (gpmA) [5.4.2.1] |
| 0794 | ribosomal protein L31 (rpmE) |
| 0795 | hypothetical protein |
| 0796 | A/G-specific adenine glycosylase (mutY) [3.2.2.—] |
| 0797 | UPF0269 protein |
| 0798 | Membrane-bound lytic murein transglycosylase C precursor(Murein hydrolase C) (mltC) [3.2.1.—] |
| 0799 | Ser/Thr protein phosphatase superfamily |
| 0800 | nicotinamide-nucleotide adenylyltransferase [2.7.7.1] |
| 0801 | hypothetical protein |
| 0802 | 3,4-dihydroxy-2-butanone 4-phosphate synthase (ribB) |
| 0803 | Lipooligosaccharide biosynthesis protein lpsA [2.—.—.—] |
| 0804 | RNA methyltransferase, TrmH family, group 2 |
| 0805 | methyltransferase, putative |
| 0806 | Cell division protein ftsY homolog (ftsY) |
| 0807 | Cell division ATP-binding protein ftsE (ftsE) |
| 0808 | putative protein insertion permease FtsX (ftsX) |
| 0809 | Acetyl-CoA acetyltransferase (Acetoacetyl-CoA thiolase) (atoB) [2.3.1.9] |
| 0810 | membrane protein, putative |
| 0811 | Acetate CoA-transferase beta subunit (Acetyl-CoA: acetoacetate CoA transferase beta subunit) (atoA) [2.8.3.8] |
| 0812 | Acetate CoA-transferase alpha subunit (Acetyl-CoA: acetoacetate CoA transferase alpha subunit) [2.8.3.8] |
| 0813 | Putative HTH-type transcriptional regulator |
| 0814 | ribosomal protein S10 (rpsJ) |
| 0815 | ribosomal protein L3 (rplC) |
| 0816 | ribosomal protein L4/L1 family (rplD) |
| 0817 | ribosomal protein L23 (rplW) |
| 0818 | ribosomal protein L2 (rplB) |
| 0819 | ribosomal protein S19 (rpsS) |
| 0820 | ribosomal protein L22 (rplV) |
| 0821 | ribosomal protein S3 (rpsC) |
| 0822 | ribosomal protein L16 (rplP) |
| 0823 | ribosomal protein L29 (rpmC) |
| 0824 | ribosomal protein S17 (rpsQ) |
| 0825 | conserved hypothetical protein |
| 0826 | ribosomal protein L14 (rplN) |
| 0827 | ribosomal protein L24 (rplX) |
| 0828 | ribosomal protein (rpL5) |
| 0829 | ribosomal protein S14p/S29e (rpsN) |
| 0830 | ribosomal protein S8 (rpsH) |
| 0831 | ribosomal protein (rpL6) |
| 0832 | ribosomal protein L18 (rplR) |
| 0833 | ribosomal protein S5 (rpsE) |
| 0834 | ribosomal protein L30 (rpmD) |
| 0835 | ribosomal protein L15 (rplO) |
| 0836 | Preprotein translocase secY subunit (secY) |
| 0837 | ribosomal protein L36 (rpmJ) |
| 0838 | lipoprotein, putative |
| 0839 | ribosomal protein S13p/S18e (rpsM) |
| 0840 | 2,3-bisphosphoglycerate-dependent phosphoglycerate mutase(Phosphoglyceromutase) (PGAM) (BPG-dependent |
| | PGAM) (dPGM) (gpmA) [5.4.2.1] |
| 0841 | Preprotein translocase secY subunit (secY) |
| 0842 | ribosomal protein L36 (rpmJ) |
| 0843 | ribosomal protein S13p/S18e (rpsM) |
| 0844 | ribosomal protein S11 (rpsK) |
| 0845 | ribosomal protein (rpS4) |
| 0846 | ribosomal protein S11 (rpsK) |
| 0847 | ribosomal protein S4 (rpsD) |
| 0848 | DNA-directed RNA polymerase, alpha subunit (rpoA) [2.7.7.6] |
| 0849 | ribosomal protein L17 (rplQ) |
| 0850 | cyclic nucleotide-binding domain protein |
| 0851 | arylsulfatase regulator (YDEM) [1.—.—.—] |
| 0852 | Domain of unknown function, putative |
| 0853 | 1-deoxy-D-xylulose 5-phosphate reductoisomerase (dxr) [1.1.1.267] |
| 0854 | ribosome recycling factor (frr) |
| 0855 | phosphoenolpyruvate carboxykinase (ATP) (pckA) [4.1.1.49] |
| 0856 | 33 kDa chaperonin (Heat shock protein 33 homolog) (HSP33) (HSP33) |
| 0857 | hypothetical protein |
| 0858 | argininosuccinate lyase (argH) [4.3.2.1] |
| 0859 | UTP-glucose-1-phosphate uridylyltransferase (galU) [2.7.7.9] |
| 0860 | carbon storage regulator (csrA) |
| 0861 | alanyl-tRNA synthetase (alaS) [6.1.1.7] |
| 0862 | Universal stress protein A homolog (uspA) |
| 0863 | Xaa-Pro aminopeptidase (X-Pro aminopeptidase)(Aminopeptidase P II) (APP-II) (Aminoacylproline |
| | aminopeptidase) (pepP) [3.4.11.9] |
| 0864 | Hypothetical UPF0149 protein |
| 0865 | Aldose 1-epimerase (Mutarotase) (galM) [5.1.3.3] |
| 0866 | galactokinase (galK) [2.7.1.6] |
| 0867 | galactokinase (galK) [2.7.1.6] |
| 0868 | galactose-1-phosphate uridylyltransferase (galT) [2.7.7.10] |
| 0869 | hypothetical protein |
| 0870 | hypothetical protein |
| 0871 | galactose repressor (galR) |
| 0872 | D-galactose-binding protein (mglB) |
| 0873 | Galactoside transport ATP-binding protein mglA (mglA) |
| 0874 | Galactoside transport system permease protein mglC (mglC) |
| 0875 | membrane protein, putative |
| 0876 | intracellular septation protein A (ispZ) |
| 0877 | acyl CoA thioester hydrolase family protein NMB0925 [3.1.2.—] |
| 0878 | Protein (AB020211) |
| 0879 | Putative soluble lytic murein transglycosylase precursor [3.2.1.—] |
| 0880 | soluble lytic murein transglycosylase (Slt70) [3.2.1.—] |
| 0881 | trp operon repressor (trpR) |
| 0882 | monofunctional biosynthetic peptidoglycan transglycosylase (mtgA) [2.4.2.—] |
| 0883 | Fumarate reductase subunit D (frdD) [1.3.99.1] |
| 0884 | Fumarate reductase subunit C (frdC) [1.3.99.1] |
| 0885 | Fumarate reductase iron-sulfur protein (frdB) [1.3.99.1] |
| 0886 | fumarate reductase, flavoprotein subunit (frdA) [1.3.99.1] |
| 0887 | lysyl-tRNA synthetase-related protein GenX |
| 0888 | Transcriptional regulatory protein cpxR homolog (cpxR) |
| 0889 | small protein A |
| 0890 | 37 kDa nucleoid-associated protein homolog |
| 0891 | Protein of unknown function (DUF1414) superfamily |
| 0892 | predicted hydrolase |
| 0893 | conserved hypothetical protein |
| 0894 | molybdopterin-guanine dinucleotide biosynthesis protein A (mob) |
| 0895 | Protein yihD (o89) |
| 0896 | Thiol: disulfide interchange protein dsbA precursor (por) [5.3.4.1] |
| 0897 | Protein HI0847 (ORF3) (YIFE) |
| 0898 | tRNA (uracil-5-)-methyltransferase (trmA) [2.1.1.35] |
| 0899 | gtg start, alternate starts possible |
| 0900 | sigma-E factor regulatory protein RseC STY2830 |
| 0901 | molybdopterin-guanine dinucleotide biosynthesis protein B (mobB) |
| 0902 | drug resistance translocase family protein NMB1435 (Cereon) |
| 0903 | heme-binding lipoprotein precursor hbpA [validated] (dppA) |
| 0904 | Protein |
| 0905 | conserved protein |
| 0906 | DNA polymerase I (POL I) (polA) [2.7.7.7] |
| 0907 | Family of unknown function (DUF710) superfamily |
| 0908 | Protein |
| 0909 | ClpB protein (clpB) [3.4.21.—] |
| 0910 | RNA methyltransferase, TrmH family, group 3 |
| 0911 | ribonuclease R.(rnr) [3.1.—.—] |
| 0912 | conserved hypothetical integral membrane protein subfamily |
| 0913 | pyridoxamine 5′-phosphate oxidase (pdxH) [1.4.3.5] |
| 0914 | GTP-binding protein TypA (typA) |
| 0915 | glutamine synthetase, type I (glnA) [6.3.1.2] |
| 0916 | Wzz homolog (WZZE) |
| 0917 | conserved hypothetical protein |
| 0918 | glycosyl transferase, group 2 family protein (partial) [2.—.—.—] |
| 0919 | HIO869 |
| 0920 | membrane protein, putative |
| 0921 | HIO871 |
| 0922 | HIO872 (rfbP) [2.—.—.—] |
| 0923 | dTDP-glucose 4,6-dehydratase (rfbB) [4.2.1.46] |
| 0924 | O-Antigen Polymerase family |
| 0925 | 3.4.11.23 (pepA) [3.4.11.23] |
| 0926 | Nucleoside diphosphate kinase (NDK) (NDP kinase)(Nucleoside-2-P kinase) (ndk) [2.7.4.6] |
| 0927 | GTP1/Obg family protein (F390) |
| 0928 | Hypothetical transport protein |
| 0929 | ribosomal protein L27 (rpmA) |
| 0930 | ribosomal protein L21 (rpiU) |
| 0931 | Octaprenyl-diphosphate synthase (Octaprenyl pyrophosphatesynthetase) (OPP synthetase) (ispB) [2.5.1.—] |
| 0932 | Protein |
| 0933 | Na(+)-linked D-alanine glycine permease (alanine) |
| 0934 | Aerobic respiration control protein arcA homolog (arcA) |
| 0935 | Thiol: disulfide interchange protein dsbD precursor(Protein-disulfide reductase) (Disulfide reductase) (C-type |
| | cytochromebiogenesis protein cycZ) (dsbD) [1.8.1.8] |
| 0936 | DoxD-like family |
| 0937 | bifunctional purine biosynthesis protein PurH (purH) |
| 0938 | phosphoribosylamine-glycine ligase (purD) [6.3.4.13] |
| 0939 | serine hydroxymethyltransferase (glyA) [2.1.2.1] |
| 0940 | dephospho-CoA kinase (coaE) [2.7.1.24] |
| 0941 | Domain of unknown function (DUF329) superfamily |
| 0942 | 3.6.1.— (rhlB) [3.6.1.—] |
| 0943 | transcriptional regulator (Bm3R1) |
| 0944 | membrane-fusion protein |
| 0945 | AcrB (acrB) |
| 0946 | cell division protein FtsN (ftsN) |
| 0947 | Multidrug resistance protein B homolog (emrB) |
| 0948 | Multidrug resistance protein A homolog (emrA) |
| 0949 | dihydrofolate reductase (folA) [1.5.1.3] |
| 0950 | glutamate 5-kinase (proB) [2.7.2.11] |
| 0951 | (Di)nucleoside polyphosphate hydrolase (invA) [3.6.1.—] |
| 0952 | Predicted permease |
| 0953 | prolipoprotein diacylglyceryl transferase (lgt) [2.4.99.—] |
| 0954 | thymidylate synthase (thyA) [2.1.1.45] |
| 0955 | cytidine/deoxycytidylate deaminase family protein |
| 0956 | conserved hypothetical protein |
| 0957 | conserved hypothetical protein |
| 0958 | preprotein translocase, SecA subunit (secA) |
| 0959 | Mutator mutT protein (7,8-dihydro-8-oxoguanine-triphosphatase)(8-oxo-dGTPase) (dGTP pyrophosphohydrolase) |
| | (mutT) [3.6.1.—] |
| 0960 | Glutathione-regulated potassium-efflux system protein (K(+)/H(+)antiporter) (kefC) |
| 0961 | possible ubiquinone/menaquinone biosynthesis methyltransferase [2.1.1.—] |
| 0962 | ribosomal protein S2 (rpsB) |
| 0963 | translation elongation factor Ts (tsf) |
| 0964 | UDP-3-O-[3-hydroxymyristoyl] glucosamine N-acyltransferase (lpxD) [2.3.1.—] |
| 0965 | Outer membrane protein 26 precursor |
| 0966 | Protective surface antigen D15 precursor (80 kDa D15 antigen)(D-15-Ag) (Outer membrane protein D15) (D15) |
| 0967 | membrane-associated zinc metalloprotease, putative |
| 0968 | phosphatidate cytidylyltransferase (cdsA) [2.7.7.41] |
| 0969 | undecaprenyl diphosphate synthase (uppS) [2.5.1.31] |
| 0970 | leucyl-tRNA synthetase (leuS) [6.1.1.4] |
| 0971 | possible rare lipoprotein B (rlpB) |
| 0972 | DNA polymerase III, delta subunit (holA) [2.7.7.7] |
| 0973 | hypothetical protein |
| 0974 | Eag0007 (AF269166) |
| 0975 | unnamed protein product; Highly similar to stability protein StbD of Morganella morganii |
| 0976 | hypothetical protein |
| 0977 | Fels-2 prophage protein |
| 0978 | glycyl-tRNA synthetase, beta subunit (glyS) [6.1,1.14] |
| 0979 | similar to E. coli ORF, encoded by GenBank Accession Number X97282; and to H. influenzae protein HI0925, |
| | encoded by GenBank Accession Number U32774; and to H. influenzae protein HI1162, encoded by GenBank |
| | Accession Number U32796 |
| 0980 | conserved hypothetical protein |
| 0981 | glycyl-tRNA synthetase, alpha subunit (glyQ) [6.1.1.14] |
| 0982 | Catalase (hktE) [1.11.1.6] |
| 0983 | synthetase/amidase (orfa) |
| 0984 | lipoprotein, putative |
| 0985 | conserved hypothetical protein |
| 0986 | enolase (eno) [4.2.1.11] |
| 0987 | conserved hypothetical protein TIGR00275 |
| 0988 | Formate-dependent nitrite reductase complex nrfFG subunit precursor (nrfF) |
| 0989 | thiol: disulfide interchange protein DsbE (dsbE) |
| 0990 | cytochrome c-type biogenesis protein CcmF (ccmF) |
| 0991 | Inositol-1-monophosphatase (IMPase) (Inositol-1-phosphatase) (I-1-Pase) (suhB) [3.1.3.25] |
| 0992 | conserved hypothetical protein |
| 0993 | conserved hypothetical protein |
| 0994 | conserved hypothetical protein |
| 0995 | conserved hypothetical protein |
| 0996 | exodeoxyribonuclease V, gamma subunit (recC) [3.1.11.5] |
| 0997 | conserved hypothetical protein TIGR00244 |
| 0998 | riboflavin biosynthesis protein RibD (ribD) |
| 0999 | periplasmic serine protease DegS (degS) [3.4.21.—] |
| 1000 | formamidopyrimidine-DNA glycosylase (mutM) [3.2.2.23] |
| 1001 | L-2,4-diaminobutyrate decarboxylase (DABA decarboxylase)(DABA-DC) [4.1.1.—] |
| 1002 | PIN (PilT N terminus) domain (vapC) |
| 1003 | possible virulence-associated protein (vapB) |
| 1004 | Diaminobutyrate--2-oxoglutarate aminotransferase (L-diaminobutyric acid transaminase) (Diaminobutyrate |
| | transaminase) (DABAaminotransferase) (DABA-AT) (L-2,4-diaminobutyrate: 2-ketoglutarate 4-aminotransferase) |
| | (DABA-AT) [2.6.1.76] |
| 1005 | ribosomal protein L33 (rpmG) |
| 1006 | ribosomal protein-related protein |
| 1007 | DNA repair protein radC homolog (radC) |
| 1008 | phosphopantothenoylcysteine decarboxylase/phosphopantothenate-cysteine ligase (coaBC) |
| 1009 | Deoxyuridine 5′-triphosphate nucleotidohydrolase(dUTPase) (dUTP pyrophosphatase) (dut) [3.6.1.23] |
| 1010 | Ttk protein homolog (ttk) |
| 1011 | Uncharacterised protein family (UPF0270) family |
| 1012 | Catabolite gene activator (cAMP receptor protein) (cAMP-regulatoryprotein) (crp) |
| 1013 | 23S rRNA (uracil-5-)-methyltransferase RumB (rumB) [2.1.1.—] |
| 1014 | Beta-hexosaminidase (N-acetyl-beta-glucosaminidase)(Beta-N-acetylhexosaminidase) (exolI) [3.2.1.52] |
| 1015 | lipoprotein, putative |
| 1016 | histidine triad protein homolog (Ap4A) |
| 1017 | isoleucyl-tRNA synthetase (ileS) [6.1.1.5] |
| 1018 | riboflavin biosynthesis protein RibF (ribF) |
| 1019 | integral membrane protein MviN (mviN) |
| 1020 | ribosomal protein S20 (rpsT) |
| 1021 | conserved hypothetical protein |
| 1022 | naphthoate synthase (menB) [4.1.3.36] |
| 1023 | o-succinylbenzoic acid (OSB) synthetase (menC) [4.2.1.—] |
| 1024 | 3-dehydroquinate dehydratase, type II (aroQ) [4.2.1.10] |
| 1025 | acetyl-CoA carboxylase, biotin carboxyl carrier protein (accB) |
| 1026 | acetyl-CoA carboxylase, biotin carboxylase (accC) [6.4.1.2] |
| 1027 | Eag0010 |
| 1028 | Protein of unknown function (DUF560) family |
| 1029 | Sodium/pantothenate symporter |
| 1030 | sodium/pantothenate symporter (panF) |
| 1031 | Tou6 (DMT) |
| 1032 | cell filamentation protein (fic) |
| 1033 | ribosomal protein L11 methyltransferase (prmA) [2.1.1.—] |
| 1034 | tRNA-dihydrouridine synthase B (nifR3) [1.—.—.—] |
| 1035 | DNA-binding protein fis (fis) |
| 1036 | SsrA-binding protein (smpB) |
| 1037 | Phosphofructokinase |
| 1038 | lipoprotein, putative |
| 1039 | UPF0246 protein yaaA (ECO110K) |
| 1040 | Smf protein (DNA processing chain A) (dprA) |
| 1041 | 2-isopropylmalate synthase (leuA) [2.3.3.13] |
| 1042 | 3-isopropylmalate dehydrogenase (leuB) [1.1.1.85] |
| 1043 | 3-isopropylmalate dehydratase, large subunit (leuC) [4.2.1.33] |
| 1044 | 3-isopropylmalate dehydratase, small subunit (leuD) [4.2.1.33] |
| 1045 | Immunoglobulin A1 protease precursor (IGA1 protease) (iga1) [3.4.21.72] |
| 1046 | DNA replication and repair protein recF (recF) |
| 1047 | DNA polymerase III, beta subunit (dnaN) [2.7.7.7] |
| 1048 | chromosomal replication initiator protein DnaA (dnaA) |
| 1049 | transferrin-binding protein 1 precursor (tbp1) |
| 1050 | transferrin-binding protein 2 precursor (tbp2) |
| 1051 | Protein of unknown function (DUF560) family |
| 1052 | ribosomal protein L34 (rpmH) |
| 1053 | ribonuclease P protein component (rnpA) [3.1.26.5] |
| 1054 | conserved hypothetical protein TIGR00278 |
| 1055 | Inner membrane protein oxaA |
| 1056 | tRNA modification GTPase TrmE (trmE) |
| 1057 | 5.2.1.8 [5.2.1.8] |
| 1058 | Sulfatase domain protein |
| 1059 | lipoprotein signal peptidase (lspA) [3.4.23.36] |
| 1060 | 4-hydroxy-3-methylbut-2-enyl diphosphate reductase (ispH) [1.17.1.2] |
| 1061 | hypothetical protein |
| 1062 | Protein HI1008 precursor |
| 1063 | DeoR-family trancriptional regulator STY3044 (glpR) |
| 1064 | 3-hydroxyisobutyrate dehydrogenase (TSAR) [1.1.—.—] |
| 1065 | tRNA synthase-like protein |
| 1066 | L-fuculose-1-phosphate aldolase-like protein (fucA) [4.1.2.17] |
| 1067 | hydroxypyruvate isomerase [5.3.1.22] |
| 1068 | 4-hydroxybutyrate dehydrogenase [1.1.1.61] |
| 1069 | GntP (gntP) |
| 1070 | Putative cyclase superfamily |
| 1071 | hypothetical protein |
| 1072 | glycerol uptake facilitator (glpF) |
| 1073 | protein V6 (insertion sequence IS1016) |
| 1074 | ISPsy8, transposase OrfA, putative |
| 1075 | IS3-family transposase, OrfB (orfB) |
| 1076 | ISPsy9, transposase OrfB (orfB) |
| 1077 | HcsB |
| 1078 | HcsA″″ |
| 1079 | Bcs4 |
| 1080 | Bcs3 |
| 1081 | unnamed protein product; orf2 |
| 1082 | CDP-ribitol pyrophosphorylase [1.—.—.—] |
| 1083 | CDP-ribitol pyrophosphorylase (putative) [2.7.7.60] |
| 1084 | BexD (AF067140) |
| 1085 | BexC |
| 1086 | Capsule polysaccharide export inner-membrane protein bexB (bexB) |
| 1087 | ATP-binding protein bexA [3.6.3.38] |
| 1088 | thiamine ABC transporter, periplasmic binding protein (tHIB) |
| 1089 | thiamine ABC transporter, permease protein (thiP) |
| 1090 | Thiamine transport ATP-binding protein thiQ [3.6.3.25] |
| 1091 | biotin synthase (bioB) [2.8.1.6] |
| 1092 | transketolase (tkt) [2.2.1.1] |
| 1093 | Protein ahpA precursor (smp-like) |
| 1094 | Phosphoserine phosphatase (PSP) (O-phosphoserinephosphohydrolase) (PSPase) (serB) [3.1.3.3] |
| 1095 | UPF0234 protein |
| 1096 | magnesium and cobalt transport protein CorA (corA) |
| 1097 | Predicted integral membrane protein |
| 1098 | YafJ |
| 1099 | hypothetical protein |
| 1100 | hypothetical protein |
| 1101 | Helix-turn-helix domain protein |
| 1102 | hypothetical protein |
| 1103 | hypothelical protein |
| 1104 | hypothetical protein |
| 1105 | ferredoxin-type protein NapF (napF) |
| 1106 | Cytoplasmic chaperone TorD family |
| 1107 | DMSO reductase anchor subunit (DmsC) (dmsC) [1.8.99.—] |
| 1108 | Anaerobic dimethyl sulfoxide reductase chain B (DMSO reductase iron-sulfur subunit) (dmsB) [1.8.—.—] |
| 1109 | Anaerobic dimethyl sulfoxide reductase chain A precursor(DMSO reductase) (dmsA) [1.8.99.—] |
| 1110 | Protein HI1048 precursor |
| 1111 | MerT (merT) |
| 1112 | MerP (merP) |
| 1113 | ABC transporter, ATP-binding protein NMB0264 (AF035964) |
| 1114 | MtrA (AJ233398) |
| 1115 | carboxymuconolactone decarboxylase family protein |
| 1116 | restriction modification system-R protein |
| 1117 | restriction modification system-R protein [3.1.21.5] |
| 1118 | modification methylase LlaFI (methyltransfera) [2.1.1.72] |
| 1119 | ribonuclease HII (rnhB) [3.1.26.4] |
| 1120 | lipid-A-disaccharide synthase (lpxB) [2.4.1.182] |
| 1121 | acyl-[acyl-carrier-protein]--UDP-N-acetylglucosamine O-acyltransferase (lpxA) [2.3.1.129] |
| 1122 | beta-hydroxyacyl-(acyl-carrier-protein) dehydratase FabZ (fabZ) [4.2.1.—] |
| 1123 | Dca |
| 1124 | uridylate kinase (pyrH) [2.7.4.—] |
| 1125 | NrfD protein homolog (nrfD) [1.—.—.—] |
| 1126 | NrfC protein homolog precursor (nrfC) [1.—.—.—] |
| 1127 | Cytochrome c-type protein nrfB precursor (nrfB) |
| 1128 | Cytochrome c-552 precursor (Ammonia-forming cytochrome cnitrite reductase) (Cytochrome c nitrite reductase) |
| | (nrfA) [1.7.2.2] |
| 1129 | ATP-dependent helicase HrpA (hrpA) |
| 1130 | Uncharacterized small membrane protein |
| 1131 | Protein of unknown function (DUF441) superfamily |
| 1132 | conserved hypothetical protein |
| 1133 | cytochrome d ubiquinol oxidase, subunit II (cydB) [1.10.3.—] |
| 1134 | CydA (cydA) [1.10.3.—] |
| 1135 | CTP synthase (pyrG) [6.3.4.2] |
| 1136 | PnuC transporter (pnuC) |
| 1137 | PnuC transporter |
| 1138 | hypothetical protein |
| 1139 | amino acid ABC transporter, ATP-binding protein NMB0789 (ABC) |
| 1140 | amino-acid ABC transporter permease protein (permease) |
| 1141 | amino acid ABC transporter, periplasmic amino acid-binding protein NMB0787 |
| 1142 | UDP-N-acetylglucosamine 1-carboxyvinyltransferase (murA) [2.5.1.7] |
| 1143 | Protein |
| 1144 | STAS domain, putative |
| 1145 | Protein HI1084 precursor |
| 1146 | VpsC |
| 1147 | unnamed protein product; Highly similar to ABC transporter, permease protein YrbE of Escherichia coli |
| 1148 | ABC transporter ATP binding protein |
| 1149 | superoxide dismutase (Mn) (sodA) [1.15.1.1] |
| 1150 | heme exporter protein CcmA (ccmA) |
| 1151 | heme exporter protein CcmB (ccmB) |
| 1152 | Heme exporter protein C (Cytochrome c-type biogenesis protein ccmC) (ccmC) |
| 1153 | Heme exporter protein D (Cytochrome c-type biogenesis protein ccmD)-related protein |
| 1154 | Cytochrome c-type biogenesis protein ccmE (ccmE) |
| 1155 | cytochrome c-type biogenesis protein CcmF (ccmF) |
| 1156 | Thiol: disulfide interchange protein dsbE (Cytochrome c biogenesisprotein ccmG) (dsbE) |
| 1157 | CcmH (nrfF) |
| 1158 | CcmH (nrfF) |
| 1159 | conserved hypothetical protein |
| 1160 | lipoprotein, putative |
| 1161 | DNA ligase, NAD-dependent (ligA) [6:5.1.2] |
| 1162 | cell division protein ZipA (zipA) |
| 1163 | CysZ protein homolog (cysZ) |
| 1164 | cysteine synthase A (cysK) [2.5.1.47] |
| 1165 | Transporter, MFS superfamily (MFS) |
| 1166 | lipopolysaccharide heptosyltransferase II (rfaF) |
| 1167 | Xylose operon regulatory protein (xylR) |
| 1168 | Na+/H+ antiporter NhaC (nhaC) |
| 1169 | aminotransferase [2.6.1.—] |
| 1170 | Xylose transport system permease protein xylH (xylH) |
| 1171 | D-xylose transport ATP-binding protein xylG (xylG) |
| 1172 | D-xylose-binding periplasmic protein precursor (xylF) |
| 1173 | xylose isomerase (xylA) [5.3.1.5] |
| 1174 | xylulokinase (xylB) [2.7.1.17] |
| 1175 | ADP-L-glycero-D-manno-heptose-6-epimerase (rfaD) [5.1.3.20] |
| 1176 | Thioredoxin-like protein |
| 1177 | deoxyribose-phosphate aldolase (deoC) [4.1.2.4] |
| 1178 | Mg chelatase-related protein |
| 1179 | conserved possible cell division GTP-binding protein |
| 1180 | LapB (lapB) |
| 1181 | Oligopeptide transport ATP-binding protein oppF (oppF) |
| 1182 | Oligopeptide transport ATP-binding protein oppD (oppD) |
| 1183 | Oligopeptide transport system permease protein oppC (oppC) |
| 1184 | Oligopeptide transport system permease protein oppB (oppB) |
| 1185 | Periplasmic oligopeptide-binding protein precursor (oppA) |
| 1186 | transaldolase (talB) [2.2.1.2] |
| 1187 | transaldolase (talB) [2.2.1.2] |
| 1188 | carbon starvation protein |
| 1189 | mraZ protein (mraZ) |
| 1190 | S-adenosyl-methyltransferase MraW (mraW) [2.1.1.—] |
| 1191 | cell division protein FtsL (ftsL) |
| 1192 | Peptidoglycan synthetase ftsI (Peptidoglycanglycosyltransferase 3) (Penicillin-binding protein 3) (PBP-3) (ftsI) |
| | [2.4.1.129] |
| 1193 | UDP-N-acetylmuramoylalanyl-D-glutamate--2,6-diaminopimelate ligase(EC 6.3.2.13) (UDP-N-acetylmuramyl- |
| | tripeptide synthetase) (Meso-diaminopimelate-adding enzyme) (UDP-MurNAc-tripeptide synthetase) (MurE) |
| | [6.3.2.13] |
| 1194 | UDP-N-acetylmuramoyl-tripeptide-D-alanyl-D-alanine ligase(EC 6.3.2.10) (UDP-MurNAc-pentapeptide synthetase) |
| | (D-alanyl-D-alanine-adding enzyme) (murF) [6.3.2.10] |
| 1195 | phospho-N-acetylmuramoyl-pentapeptide-transferase (mraY) [2.7.8.13] |
| 1196 | UDP-N-acetylmuramoylalanine--D-glutamate ligase (murD) [6.3.2.9] |
| 1197 | Cell division protein ftsW (ftsW) |
| 1198 | UDP-N-acetylglucosamine--N-acetylmuramyl-(pentapeptide) pyrophosphoryl-undecaprenol N-acetylglucosamine |
| | transferase (murG) [2,4,1,—] |
| 1199 | UDP-N-acetylmuramate--alanine ligase (murC) [6.3.2.8] |
| 1200 | D-alanine--D-alanine ligase (D-alanylalanine synthetase)(D-Ala-D-Ala ligase) (ddlB) [6.3.2.4] |
| 1201 | Cell division protein ftsQ homolog (ftsQ) |
| 1202 | cell division protein FtsA (ftsA) |
| 1203 | cell division protein FtsZ (ftsZ) |
| 1204 | UDP-3-O-acyl N-acetylglucosamine deacetylase (lpxC) [3.5.1.—] |
| 1205 | P-protein [Includes: Chorismate mutase (CM); Prephenatedehydratase (EC 4.2.1.51) (PDT)] (PDT) [5.4.99.5] |
| 1206 | unnamed protein product; ORF193 peptide fragment (AA 1-192) (1524 is 2nd base in codon) (P-loop) |
| 1207 | PTS IIA-like nitrogen-regulatory protein PtsN (ptsN) |
| 1208 | ABC transporter, ATP-binding protein |
| 1209 | Protein HI1149 precursor |
| 1210 | unnamed protein product; Similar to YrbK precursor protein of Escherichia coli |
| 1211 | Protein of unknown function (DUF615) superfamily |
| 1212 | PmbA protein homolog (pmbA) |
| 1213 | hypoxanthine phosphoribosyltransferase (hpt) [2.4.2.8] |
| 1214 | conserved hypothetical protein |
| 1215 | kinase-like protein (gltP) |
| 1216 | Anaerobic ribonucleoside-triphosphate reductase activating protein(EC 1.97.1.4) (Class III anaerobic ribonucleotide |
| | reductase smallcomponent) (nrdG) [1.97.1.4] |
| 1217 | Transport ATP-binding protein cydC (cydC) |
| 1218 | Transport ATP-binding protein cydD (cydD) |
| 1219 | thioredoxin-disulfide reductase (trxB) [1.8.1.9] |
| 1220 | Protein |
| 1221 | ferrochelatase (hemH) [4.99.1.1] |
| 1222 | uncharacterized protein conserved in bacteria |
| 1223 | Protein |
| 1224 | Outer membrane protein P5 precursor (OMP P5) (ompA) |
| 1225 | glutaredoxin-related protein |
| 1226 | histidinol-phosphate aminotransferase (hisC) [2.6.1.9] |
| 1227 | phosphoserine aminotransferase (serC) [2.6.1.52] |
| 1228 | UPF0265 protein |
| 1229 | chorismate binding enzyme [4.1.3.—] |
| 1230 | p-aminobenzoate synthase component I (pabB) [4.1.3.—] |
| 1231 | TrpG (trpG) [4.1.3.27] |
| 1232 | S-adenosylmethionine synthetase (metK) [2.5.1.6] |
| 1233 | Protein sprT (sprT) |
| 1234 | OPA protein |
| 1235 | conserved hypothetical protein |
| 1236 | Arginine transport system permease protein artM (artM) |
| 1237 | Arginine transport system permease protein artQ (artQ) |
| 1238 | Arginine-binding periplasmic protein precursor (artI) |
| 1239 | Arginine transport ATP-binding protein artP (artP) |
| 1240 | phosphoheptose isomerase (gmhA) |
| 1241 | hypothetical protein |
| 1242 | DNA ligase (Polydeoxyribonucleotide synthase [ATP]) [6.5.1.1] |
| 1243 | Dipeptide transport ATP-binding protein dppF (dppF) |
| 1244 | Dipeptide transport ATP-binding protein dppD (dppD) |
| 1245 | Dipeptide transport system permease protein dppC (dppC) |
| 1246 | Dipeptide transport system permease protein dppB (dppB) |
| 1247 | hypothetical protein |
| 1248 | hypothetical protein |
| 1249 | DNA helicase II (uvrD) [3.6.1.—] |
| 1250 | Vng6305c |
| 1251 | 6-pyruvoyl tetrahydropterin synthase, putative [4.2.3.12] |
| 1252 | exsB protein |
| 1253 | lipoprotein, putative |
| 1554 | Eag0009 |
| 1255 | Eag0010 |
| 1256 | Eag0011 |
| 1257 | branched-chain amino acid aminotransferase (ilvE) [2.6.1.42] |
| 1258 | Glycine cleavage system transcriptional activator homolog (gcvA) |
| 1259 | SAM-dependent methyltransferase-like protein |
| 1260 | Succinyl-CoA synthetase beta chain (SCS-beta) (sucC) [6.2.1.5] |
| 1261 | Succinyl-CoA synthetase alpha chain (SCS-alpha) (sucD) [6.2.1.5] |
| 1262 | Sua5/YciO/YrdC/YwlC family protein |
| 1263 | Ribosomal large subunit pseudouridine synthase B(Pseudouridylate synthase) (Uracil hydrolyase) [4.2.1.70] |
| 1264 | HTH-type transcriptional regulator cysB (Cys regulon transcriptionalactivator) (cysB) |
| 1265 | 2.1.1.72 [2.1.1.72] |
| 1266 | Hypothetical UPF0115 protein |
| 1267 | phosphate acetyltransferase (pta) [2.3.1.8] |
| 1268 | acetate kinase (ackA) [2.7.2.1] |
| 1269 | hypothetical protein |
| 1270 | b2295 |
| 1271 | CvpA family protein (cvpA) |
| 1272 | amidophosphoribosyltransferase (purF) [2.4.2.14] |
| 1273 | conserved hypothetical protein TIGR01777 |
| 1274 | arginine repressor (argR) |
| 1275 | malate dehydrogenase, NAD-dependent (mdh) [1.1.1.37] |
| 1276 | Lysyl-tRNA synthetase (Lysine-tRNA ligase) (LysRS) (lysU) [6.1.1.6] |
| 1277 | Lysyl-tRNA synthetase (Lysine-tRNA ligase) (LysRS) (lysU) [6.1.1.6] |
| 1278 | Peptide chain release factor 2 (RF-2) (RF) |
| 1279 | Thiol: disulfide interchange protein dsbC precursor (dsbC) [5.3.4.1] |
| 1280 | single-stranded-DNA-specific exonuclease RecJ (recJ) [3.1.—.—] |
| 1281 | DSBA-like thioredoxin domain family |
| 1282 | MTA/SAH nucleosidase |
| 1283 | TonB-dependent receptor NMB1497 (Y08983) |
| 1284 | LctP (lctP) |
| 1285 | cytidylate kinase (cmk) [2.7.4.14] |
| 1286 | ribosomal protein S1 (rpsA) |
| 1287 | integration host factor, beta subunit (ihfB) |
| 1288 | predicted membrane protein |
| 1289 | predicted N-acetylglucosaminyl transferase |
| 1290 | orotidine 5′-phosphate decarboxylase (pyrF) [4.1.1.23] |
| 1291 | translation initation factor SUI1, putative |
| 1292 | DnaA family protein (dnaA) |
| 1293 | uracil permease (uraA) |
| 1294 | hypothetical protein |
| 1295 | uracil phosphoribosyltransferase (upp) [2.4.2.9] |
| 1296 | DNA polymerase III subunit gamma/tau (dnaX) [2.7.7.7] |
| 1297 | adenine phosphoribosyltransferase (apt) [2.4.2.7] |
| 1298 | dihydrolipoamide dehydrogenase (lpdA) [1.8.1.4] |
| 1299 | pyruvate dehydrogenase complex dihydrolipoamide acetyltransferase (aceF) [2.3.1.12] |
| 1300 | Pyruvate dehydrogenase E1 component (aceE) [1.2.4.1] |
| 1301 | hypothetical protein |
| 1302 | methylglyoxal synthase (mgsA) [4.2.3.3] |
| 1303 | conserved hypothetical protein |
| 1304 | dnaK-type molecular chaperone (dnaK) |
| 1305 | heat shock protein dnaJ (dnaJ) |
| 1306 | gamma-glutamyl phosphate reductase (proA) [1.2.1.41] |
| 1307 | membrane protein |
| 1308 | membrane protein, putative |
| 1309 | Bicyclomycin resistance protein homolog (bcr) |
| 1310 | Ribosomal small subunit pseudouridine synthase A (16Spseudouridylate 516 synthase) (16S pseudouridine 516 |
| | synthase) (Uracilhydrolyase) (rsuA) [4.2.1.70] |
| 1311 | CpsH protein |
| 1312 | hypothetical protein |
| 1313 | NADP-dependent malic enzyme (NADP-ME) (oxaloacetate-de) [1.1.1.40] |
| 1314 | sulfatase |
| 1315 | UvrABC system protein B (UvrB protein) (Excinuclease ABC subunit B) (uvrB) |
| 1316 | High-affinity nickel-transport protein family |
| 1317 | Protein of unknown function (DUF1007) superfamily |
| 1318 | proteic killer suppression protein (putative) |
| 1319 | unnamed protein product; Some similarities with virulence associated protein A (vapA) |
| 1320 | ABC transporter ATP-binding protein (ABC) |
| 1321 | Invasion gene expression up-regulator, SirB superfamily |
| 1322 | similar to [SwissProt Accession Number P44140] (GNAT) |
| 1323 | conserved hypothetical protein |
| 1324 | transcription-repair coupling factor (mfd) |
| 1325 | HtrA [3.4.21.—] |
| 1326 | acetyl-CoA carboxylase, carboxyl transferase, beta subunit (accD) [6.4.1.2] |
| 1327 | Folylpolyglutamate synthase (Folylpoly-gamma-glutamatesynthetase) (FPGS) (folC) [6.3.2.17] |
| 1328 | hypothetical protein |
| 1329 | SanA protein homolog (sanA) |
| 1330 | homoserine O-acetyltransferase (metX) [2.3.1.31] |
| 1331 | DNA gyrase, A subunit (gyrA) [5.99.1.3] |
| 1332 | ycaO protein |
| 1333 | conserved hypothetical protein |
| 1334 | possible iron ABC transporter periplasmic binding protein (III) |
| 1335 | HemU (III) |
| 1336 | iron (III) |
| 1337 | conserved hypothetical protein |
| 1338 | GloB [3.1.2.6] |
| 1339 | tellurite resistance protein TehB (tehB) |
| 1340 | Methionyl-tRNA synthetase (Methionine-tRNA ligase)(MetRS) (metG) [6.1.1.10] |
| 1341 | Mrp (mrp) |
| 1342 | NAD(P)H nitroreductase [1.—.—.—] |
| 1343 | cytidine 5″″monophosphate N-acetylneuraminic acid synthetase (neuA) [2.7.7.43] |
| 1344 | YhbC-like protein |
| 1345 | Transcription elongation protein nusA (nusA) |
| 1346 | Translation initiation factor IF-2 (infB) |
| 1347 | HSDR (hsdR) [3.1.21.3] |
| 1348 | conserved hypothetical protein |
| 1349 | Prolipoprotein, putative |
| 1350 | hypothetical protein |
| 1351 | conserved hypothetical protein |
| 1352 | HsdA (hsdS) |
| 1353 | ALXA and HSDM (hsdM) [2.1.1.72] |
| 1354 | ribosome-binding factor A (rbfA) |
| 1355 | tRNA pseudouridine synthase B (tRNA pseudouridine 55synthase) (Psi55 synthase) (Pseudouridylate synthase) |
| | (Uracilhydrolyase) (truB) [4.2.1.70] |
| 1356 | T-protein [Includes: Chorismate mutase (CM); Prephenatedehydrogenase (EC 1.3.1.12) (PDH)] (PDH) [5.4.99.5] |
| 1357 | possible GTP cyclohydrolase I |
| 1358 | Zn-ribbon-containing protein |
| 1359 | possible GTP cyclohydrolase I |
| 1360 | Zn-ribbon-containing protein |
| 1361 | uncharacterized protein conserved in bacteria (orf5) |
| 1362 | Zn-ribbon-containing protein |
| 1363 | uncharacterized protein conserved in bacteria (orf5) |
| 1364 | Cysteine sulfinate desulfinase (CSD) (CSD) [2.8.1.7] |
| 1365 | micrococcal nuclease-like protein (SNase) [3.131.1] |
| 1366 | Holin-like protein cidA 2 |
| 1367 | membrane protein, putative |
| 1368 | Deoxyguanosinetriphosphate triphosphohydrolase-like protein (DGTPASE) [3.1.5.1] |
| 1369 | hypothetical protein |
| 1370 | ABC transporter ATP-binding protein uup-1 |
| 1371 | Protein yadF [4.2.1.1] |
| 1372 | asparaginyl-tRNA synthetase (asnS) [6.1.1.22] |
| 1373 | 6,7-dimethyl-8-ribityllumazine synthase (ribH) [2.5.1.9] |
| 1374 | transcription antitermination factor NusB (nusB) |
| 1375 | thiamine-monophosphate kinase (thiL) [2.7.4.16] |
| 1376 | Phosphatidylglycerophosphatase A (pgpA) [3.1.3.27] |
| 1377 | threonine efflux protein |
| 1378 | dihydrodipicolinate reductase (dapB) [1.3.1.26] |
| 1379 | unnamed protein product; Similar to ferredoxin-like protein YfaE of Escherichia coli (petF1) [1.17.1.—] |
| 1380 | conserved hypothetical protein |
| 1381 | phenylalanyl-tRNA synthetase, alpha subunit (pheS) [6.1.1.20] |
| 1382 | phenylalanyl-tRNA synthetase, beta subunit (pheT) [6.1.1.20] |
| 1383 | integration host factor, alpha subunit (ihfA) |
| 1384 | lipoprotein (nlpC) |
| 1385 | conserved hypothetical protein |
| 1386 | Putative 5′(3′)-deoxyribonucleotidase (dNT) [3.1.3.—] |
| 1387 | NAD-dependent deacetylase (Regulatory protein SIR2homolog) (DMB) [3.5.1.—] |
| 1388 | conserved hypothetical protein |
| 1389 | XpsR, putative |
| 1390 | death-on-curing family protein |
| 1391 | DNA translocase ftsK |
| 1392 | transcriptional regulator, Sir2 family (DMB) [3.5.1.—] |
| 1393 | probable phosphoprotein phosphatase homolog Imo1821, putative |
| 1394 | Protein of unknown function DUF262 family |
| 1395 | PUTATIVE ATPASE PROTEIN, putative |
| 1396 | arylsulfatase A [3.1.6.—] |
| 1397 | HI1317 (fragment) |
| 1398 | translation initiation factor IF-3 (infC) |
| 1399 | ribosomal protein L35 (rpL35) |
| 1400 | ribosomal protein L20 (rplT) |
| 1401 | exodeoxyribonuclease V, beta subunit (recB) [3.1.11.5] |
| 1402 | exodeoxyribonuclease V, alpha subunit (recD) [3.1.11.5] |
| 1403 | Hypothetical UPF0268 protein |
| 1404 | Ion protease (Ion) [3.4.21.—] |
| 1405 | beta-hydroxyacyl-(acyl-carrier-protein) dehydratase FabA (fabA) [4.2.1.—] |
| 1406 | conserved hypothetical protein |
| 1407 | lipoprotein, putative |
| 1408 | ribosomal protein S15 (rpsO) |
| 1409 | D-alanyl-D-alanine carboxypeptidase/D-alanyl-D-alanine-endopeptidase (dacB) [3.4.16.4] |
| 1410 | Transcription elongation factor greA (Transcript cleavage factorgreA) (greA) |
| 1411 | conserved hypothetical protein TIGR00253 |
| 1412 | ribosomal RNA large subunit methyltransferase J (rrmJ) [2.1.1.—] |
| 1413 | Cell division protein ftsH homolog 1 (ftsH) [3.4.24.—] |
| 1414 | conserved hypothetical protein |
| 1415 | uncharacterized protein conserved in bacteria (orf5) |
| 1416 | selenocysteine lyase (CSD) [2.8.1.7] |
| 1417 | ABC transporter ATP-binding protein uup-1 |
| 1418 | ABC transporter ATP-binding protein uup-1 |
| 1419 | Cell division protein ftsH homolog 1 (ftsH) [3.4.24.—] |
| 1420 | HmcB (AP001508) |
| 1421 | HmcC [3.4.22.—] |
| 1422 | HmcD |
| 1423 | spermidine/putrescine-binding protein 1 precursor (potD) |
| 1424 | spermidine/putrescine transport system permease potC (potC) |
| 1425 | Spermidine/putrescine transport system permease protein potB (potB) |
| 1426 | Spermidine/putrescine transport ATP-binding protein potA (potA) |
| 1427 | peptidase T (pepT) [3.4.11.14] |
| 1428 | Protein (napA) |
| 1429 | cytidine deaminase (cdd) [3.5.4.5] |
| 1430 | methyltransferase, putative |
| 1431 | sodium/proline symporter (putP) |
| 1432 | Ribonuclease G (RNase G) (Cytoplasmic axial filamentprotein) (cafA) [3.1.4.—] |
| 1433 | glutaminyl-tRNA synthetase (glnS) [6.1.1.18] |
| 1434 | YcgN |
| 1435 | 4-alpha-glucanotransferase (malQ) [2.4.1.25] |
| 1436 | 1,4-alpha-glucan branching enzyme (glgB) [2.4.1.18] |
| 1437 | glycogen debranching enzyme GlgX (glgX) [3.2.1.—] |
| 1438 | glucose-1-phosphate adenylyltransferase (glgC) [2.7.7.27] |
| 1439 | Glycogen synthase (Starch [bacterial glycogen]synthase) (glgA) [2.4.1.21] |
| 1440 | hypothetical protein |
| 1441 | hypothetical protein |
| 1442 | Glycogen phosphorylase (glgP) [2.4.1.1] |
| 1443 | NAD(P) transhydrogenase, alpha subunit (pntA) [1.6.1.1] |
| 1444 | NAD(P) transhydrogenase subunit beta (Pyridinenucleotide transhydrogenase subunit beta) (Nicotinamide |
| | nucleotidetranshydrogenase subunit beta) (pntB) [1.6.1.2] |
| 1445 | Bacterial regulatory protein, LysR family (PA4174) |
| 1446 | DNA topoisomerase (topA) [5.99.1.2] |
| 1447 | acyl carrier protein phosphodiesterase (acpD) [3.1.4.14] |
| 1448 | threonyl-tRNA synthetase (thrS) [6.1.1.3] |
| 1449 | PqqL [3.4.99.—] |
| 1450 | conserved hypothetical protein |
| 1451 | MOLYBDENUM-PTERIN-BINDING PROTEIN (mopI) |
| 1452 | dissimilatory sulfite reductase, gamma subunit (dsvC) [1.8.—.—] |
| 1453 | YdaO protein |
| 1454 | killing factor kicB (kicB) |
| 1455 | Chromosome partition protein mukE (kicA) |
| 1456 | MukB (mukB) |
| 1457 | conserved hypothetical protein |
| 1458 | integral membrane protein |
| 1459 | Exodeoxyribonuclease I (Exonuclease I) (DNAdeoxyribophosphodiesterase) (dRPase) (sbcB) [3.1.11.1] |
| 1460 | Phosphate regulon sensor protein phoR (phoR) [2.7.3.—] |
| 1461 | Phosphate regulon transcriptional regulatory protein phoB (phoB) |
| 1462 | phosphate ABC transporter, ATP-binding protein (pstB) [3.6.3.27] |
| 1463 | phosphate ABC transporter, permease protein PtsA (pstA) |
| 1464 | phosphate ABC transporter, permease protein PstC (pstC) |
| 1465 | phosphate ABC transporter, phosphate-binding protein (pstS) |
| 1466 | nonheme ferritin homolog (rsgA) |
| 1467 | Ferritin like protein 2 (rsgA) |
| 1468 | possible glycosyltransferase |
| 1469 | anthranilate synthase component I (trpE) [4.1.3.27] |
| 1470 | Anthranilate synthase component II (Glutamine amido-transferase) (trpG) [4.1.3.27] |
| 1471 | Uncharacterized protein, 4-oxalocrotonate tautomerase homolog |
| 1472 | anthranilate phosphoribosyltransferase (trpD) [2.4.2.18] |
| 1473 | Tryptophan biosynthesis protein trpCF [Includes: Indole-3-glycerolphosphate synthase (IGPS); N-(5′-phospho- |
| | ribosyl)anthranilate isomerase (EC 5.3.1.24) (PRAI)] (trpC) [4.1.1.48] |
| 1474 | hydrogenase assembly chaperone HypC/HupF (hypC) |
| 1475 | valyl-tRNA synthetase (valS) [6.1.1.9] |
| 1476 | Modification methylase HindIII (Adenine-specificmethyltransferase HindIII) (M.HindIII) (hindIIIM) [2.1.1.72] |
| 1477 | Type II restriction enzyme HindIII (EndonucleaseHindIII) (R.HindIII) (hindIIIR) [3.1.21.4] |
| 1478 | UPF0267 protein |
| 1479 | conserved hypothetical protein |
| 1480 | DNA polymerase III, chi subunit (holC) [2.7.7.7] |
| 1481 | fumarate hydratase, class II (fumC) [4.2.1.2] |
| 1482 | conserved hypothetical protein |
| 1483 | Protein trpH |
| 1484 | dihydroorotate dehydrogenase (pyrD) [1.3.3.1] |
| 1485 | conserved hypothetical protein |
| 1486 | Eag0005 |
| 1487 | Eag0003 |
| 1488 | conserved hypothetical protein |
| 1489 | conserved hypothetical protein |
| 1490 | conserved hypothetical protein |
| 1491 | conserved hypothetical protein |
| 1492 | conserved hypothetical protein |
| 1493 | putative baseplate protein |
| 1494 | conserved hypothetical protein |
| 1495 | conserved hypothetical protein |
| 1496 | hypothetical protein |
| 1497 | conserved hypothetical protein |
| 1498 | conserved hypothetical protein |
| 1499 | putative tail length tape measure protein |
| 1500 | conserved hypothetical protein |
| 1501 | conserved hypothetical protein |
| 1502 | conserved hypothetical protein |
| 1503 | conserved hypothetical protein |
| 1504 | conserved hypothetical protein |
| 1505 | conserved hypothetical protein |
| 1506 | conserved hypothetical protein |
| 1507 | conserved hypothetical protein |
| 1508 | conserved hypothetical protein |
| 1509 | conserved hypothetical protein |
| 1510 | conserved hypothetical protein |
| 1511 | Protein traN |
| 1512 | phage-related protein, HI1409 family |
| 1513 | phage terminase, large subunit, PBSX family |
| 1514 | Terminase small subunit superfamily |
| 1515 | Protein of unknown function superfamily |
| 1516 | DNA-binding protein |
| 1517 | conserved hypothetical protein |
| 1518 | lytic enzyme |
| 1519 | phage holin, lambda family |
| 1520 | conserved hypothetical protein |
| 1521 | conserved hypothetical protein |
| 1522 | integrase |
| 1523 | conserved hypothetical protein |
| 1524 | Anaerobic regulatory protein (fnr) |
| 1525 | Universal stress protein E homolog |
| 1526 | Protein HI1427 precursor |
| 1527 | phosphoribosylglycinamide formyltransferase (purN) [2.1.2.2] |
| 1528 | phosphoribosylformylglycinamidine cyclo-ligase (purM) [6.3.3.1] |
| 1529 | YdfG (AB032242) [1.—.—.—] |
| 1530 | tryptophan synthase, beta subunit (trpB) [4.2.1.20] |
| 1531 | tryptophan synthase, alpha subunit (trpA) [4.2.1.20] |
| 1532 | USG-1 protein homolog (usg1) [1.2.1.—] |
| 1533 | ybaK/ebsC protein (ybaK) |
| 1534 | Cold shock-like protein cspD (cspD) |
| 1535 | Uncharacterised protein family (UPF0181) superfamily |
| 1536 | tRNA pseudouridine synthase C (Pseudouridylate synthase)(Uracil hydrolyase) (orfx) [4.2.1.70] |
| 1537 | tRNA pseudouridine synthase C (Pseudouridylate synthase)(Uracil hydrolyase) [4.2.1.70] |
| 1538 | Thiamine biosynthesis protein thiI (thiI) |
| 1539 | exodeoxyribonuclease VII, small subunit (xseB) [3.1.11.6] |
| 1540 | Geranyltranstransferase (Farnesyl-diphosphate synthase)(FPP synthase) (ispA) [2.5.1.10] |
| 1541 | 1-deoxy-D-xylulose-5-phosphate synthase (dxs) [2.2.1.7] |
| 1542 | transcriptional regulator |
| 1543 | Stringent starvation protein B homolog (sspB) |
| 1544 | Stringent starvation protein A homolog (sspA) |
| 1545 | ribosomal protein S9 (rpsI) |
| 1546 | ribosomal protein L13 (rplM) |
| 1547 | 5,10-methylenetetrahydrofolate reductase (metF) [1.7.99.5] |
| 1548 | dethiobiotin synthetase (bioD) [6.3.3,3] |
| 1549 | Uncharacterized protein conserved in bacteria |
| 1550 | GTP cyclohydrolase I (folE) [3.5.4.16] |
| 1551 | Molybdopterin biosynthesis protein moeA (moeA) |
| 1552 | Molybdopterin biosynthesis protein moeB (moeB) |
| 1553 | Hypothetical UPF0263 protein |
| 1554 | Protein HI1453 precursor (thioredoxin) [1.8.4.6] |
| 1555 | Cytochrome c-type biogenesis protein ccdA (ccdA) [4.4.1.17] |
| 1556 | Peptide methionine sulfoxide reductase msrA/msrB[Includes: Peptide methionine sulfoxide reductase msrA |
| | (Protein-methionine-S-oxide reductase) (Peptide Met(O) reductase); Peptidemethionine sulfoxide reductase msrB] |
| | (msrA) [1.8.4.6] |
| 1557 | lipoprotein, putative |
| 1558 | Protein HI1457 precursor |
| 1559 | Eag0009 |
| 1560 | unnamed protein product; Similar to transcription initiation factor sigma homolog (sigma-W) |
| 1561 | Invasin precursor (Outer membrane adhesin) |
| 1562 | hypothetical protein |
| 1563 | hypothetical protein |
| 1564 | RND efflux system, outer membrane lipoprotein, NodT family subfamily |
| 1565 | H. influenzae predicted coding region HI1462.1 (LEA) |
| 1566 | ferrichrome-iron outermembrane receptor protein |
| 1567 | Cell division protein ftsH homolog 1 (ftsH) [3.4.24.—] |
| 1568 | Cell division protein ftsH homolog 1 (ftsH) [3.4.24.—] |
| 1569 | dihydropteroate synthase (folP) [2.5.1.15] |
| 1570 | phosphoglucosamine mutase (glmM) [5.4.2.—] |
| 1571 | phosphohistidine phosphatase SixA (sixA) [3.1.3.—] |
| 1572 | Hypothetical tonB-dependent receptor HI1466.1 |
| 1573 | Hypothetical ABC transporter ATP-binding protein |
| 1574 | ABC transporter, ATP-binding protein (ALD) |
| 1575 | ribosomal protein S15 (rpsO) |
| 1576 | molybdenum-binding periplasmic protein |
| 1577 | iron (III) |
| 1578 | ABC-type iron transport system, permease component CAC1990 (III) |
| 1579 | Protein HI1472 precursor (III) |
| 1580 | modD protein (modD) |
| 1581 | FbpC (III) [3.6.3.25] |
| 1582 | NifC-like ABC-type porter |
| 1583 | molybdenum ABC transporter, periplasmic molybdate-binding protein (modA) |
| 1584 | ADP-heptose synthase (rfaE) [2.7.—.—] |
| 1585 | hypothetical protein |
| 1586 | lipid A biosynthesis lauroyl acyltransferase (htrB) [2.3.1.—] |
| 1587 | DNA topoisomerase IV, B subunit (parE) [5.99.1.—] |
| 1588 | DNA topoisomerase IV, A subunit (parC) [5.99.1.—] |
| 1589 | sodium/glutamate symporter (gltS) |
| 1590 | RimK (rimK) [6.3.2.—] |
| 1591 | Glutaredoxin, GrxA family (grxA) |
| 1592 | 3-oxoacyl-[acyl-carrier-protein] synthase I (Beta-ketoacyl-ACP synthase I) (KAS I) (fabB) [2.3.1.41] |
| 1593 | Protein of unknown function (DUF752) family |
| 1594 | LicA protein (licA) |
| 1595 | lic-1 protein B (licB) |
| 1596 | Protein licC (licC) |
| 1597 | lic-1 protein D (licD) |
| 1598 | lic-1 protein D (licD) |
| 1599 | signal peptide peptidase SppA, 67K type (sppA) [3.4.—.—] |
| 1600 | Protein ydjA [1.—.—.—] |
| 1601 | conserved hypothetical protein |
| 1602 | NAD(P)H oxidoreductase BH2748 [1.6.99.—] |
| 1603 | Na/dicarboxylate symporter |
| 1604 | ImpA (R391) [3.4.21.—] |
| 1605 | phospho-2-dehydro-3-deoxyheptonate aldolase [2.5.1.54] |
| 1606 | lipoprotein releasing system, transmembrane protein LolE (lolE) |
| 1607 | lipoprotein releasing system, ATP-binding protein (lolD) |
| 1608 | dethiobiotin synthase (bioD) [6.3.3.3] |
| 1609 | biotin biosynthesis protein BioC (bioC) |
| 1610 | Protein of unknown function (DUF452) superfamily |
| 1611 | 8-amino-7-oxononanoate synthase (bioF) [2.3.1.47] |
| 1612 | adenosylmethionine-8-amino-7-oxononanoate aminotransferase (bioA) [2.6.1.62] |
| 1613 | Lipoprotein releasing system transmembrane protein lolC |
| 1614 | lactate dehydrogenase [1.1.1.29] |
| 1615 | 3-deoxy-8-phosphooctulonate synthase (kdsA) [2.5.1.55] |
| 1616 | Protein sirB1 |
| 1617 | HemK protein homolog (M.HindHemKP) (hemK) [2.1.1.—] |
| 1618 | RDD family superfamily |
| 1619 | peptide chain release factor 1 (prfA) |
| 1620 | Protein-related protein |
| 1621 | uncharacterized protein conserved in bacteria |
| 1622 | conserved hypothetical protein |
| 1623 | conserved hypothetical protein |
| 1624 | Probable tail fiber protein (ORF31) |
| 1625 | Eag0003 |
| 1626 | conserved hypothetical protein |
| 1627 | conserved hypothetical protein |
| 1628 | Mu-like prophage FluMu protein gp46 |
| 1629 | baseplate assembly protein V, probable NMB1111 |
| 1630 | Bacteriophage Mu P protein |
| 1631 | phage virion protein, probable NMB1109, putative |
| 1632 | hypothetical protein |
| 1633 | conserved hypothetical protein |
| 1634 | probable transposase protein |
| 1635 | replication protein, putative |
| 1636 | replication protein |
| 1637 | conserved hypothetical protein |
| 1638 | regulatory protein |
| 1639 | similar to CI repressor of bacteriophage lambda |
| 1640 | hypothetical protein |
| 1641 | hypothetical protein |
| 1642 | hypothetical protein |
| 1643 | hypothetical protein |
| 1644 | Serine/threonine-protein kinase PK-1 (stoPK-1) [2.7.1.37] |
| 1645 | Protein serine/threonine phosphatases [3.1.3.—] |
| 1646 | KilA-N domain family |
| 1647 | prophage CP4-57 integrase |
| 1648 | hypothetical protein |
| 1649 | pyruvate kinase (pyk) [2.7.1.40] |
| 1650 | hypothetical protein |
| 1651 | replicative DNA helicase (dnaB) [3.6.1.—] |
| 1652 | alanine racemase (alr) [5.1.1.1] |
| 1653 | glucose-6-phosphate isomerase (pgi) [5.3.1.9] |
| 1654 | 15 kd peptidoglycan-associated outer membrane lipoprotein precursor (lpp) |
| 1655 | Hypothetical lipoprotein PM0553 precursor |
| 1656 | Protein yecM |
| 1657 | arginyl-tRNA synthetase (argS) [6.1.1.19] |
| 1658 | acetolactate synthase, small subunit (ilvN) [2.2.1.6] |
| 1659 | acetolactate synthase, large subunit, biosynthetic type (ilvB) [2.2.1.6] |
| 1660 | Na+/H+ antiporter |
| 1661 | DNA-binding protein H-NS homolog (hns) |
| 1662 | formyltetrahydrofolate deformylase (purU) [3.5.1.10] |
| 1663 | 3-phosphoshikimate 1-carboxyvinyltransferase (aroA) [2.5.1.19] |
| 1664 | ATPase-like protein (putative) |
| 1665 | outer membrane lipoprotein carrier protein LolA (lolA) |
| 1666 | DNA translocase ftsK |
| 1667 | Leucine-responsive regulatory protein (lrp) |
| 1668 | DNA repair protein RadA (radA) |
| 1669 | Rd1598 |
| 1670 | conserved hypothetical protein |
| 1671 | Protein of unknown function (DUF692) superfamily |
| 1672 | EF hand domain protein |
| 1673 | hypothetical protein |
| 1674 | Uncharacterized conserved membrane protein (COG2259) |
| 1675 | conserved hypothetical protein TIGR00153 |
| 1676 | pho4 family protein VC2442 |
| 1677 | conserved hypothetical protein |
| 1678 | tRNA nucleotidyltransferase (tRNA adenylyltransferase)(tRNA CCA-pyrophosphorylase) (CCA-adding enzyme) |
| | (cca) [2.7.7.25] |
| 1679 | outer membrane lipoprotein LolB (lolB) |
| 1680 | 4-diphosphocytidyl-2C-methyl-D-erythritol kinase (ispE) [2.7.1.148] |
| 1681 | Ribose-phosphate pyrophosphokinase (RPPK) (Phosphoribosylpyrophosphate synthetase) (P-Rib-PP synthetase) |
| | (PRPP synthetase) (prsA) [2.7.6.1] |
| 1682 | tyrosyl-tRNA synthetase (tyrS) [6.1.1.1] |
| 1683 | sugar fermentation stimulation protein (sfsA) |
| 1684 | Multidrug resistance protein NorM |
| 1685 | riboflavin synthase, alpha subunit (ribE) [2.5.1.9] |
| 1686 | Aminopeptidase N (Alpha-aminoacylpeptide hydrolase) (pepN) [3.4.11.2] |
| 1687 | Major fimbrial subunit precursor (Major pilin) |
| 1688 | phosphoribosylaminoimidazole carboxylase, catalytic subunit (purE) [4.1.1.21] |
| 1689 | phosphoribosylaminoimidazole carboxylase, ATPase subunit (purK) [4.1.1.21] |
| 1690 | Aspartate aminotransferase (Transaminase A) (ASPAT) (aspC) [2.6.1.1] |
| 1691 | cobalt transport ATP-binding protein CbiO (cbiO) |
| 1692 | cobalt membrane transport protein CbiQ |
| 1693 | CbiM |
| 1694 | conserved hypothetical protein |
| 1695 | Protein HI1624 precursor |
| 1696 | HTH-type transcriptional regulator zntR homolog (merR2) |
| 1697 | 29 kDa protein |
| 1698 | membrane protein, putative |
| 1699 | translation initiation inHIBitor |
| 1700 | Protein of unknown function (DUF1043) superfamily |
| 1701 | possible integral membrane protein of DedA family (dedA) |
| 1702 | Ribosomal L25p family |
| 1703 | lysine-sensitive aspartokinase III [2.7.2.4] |
| 1704 | adenylosuccinate synthetase (purA) [6.3.4.4] |
| 1705 | 2,3,4,5-tetrahydropyridine-2,6-dicarboxylate N-succinyltransferase (dapD) [2.3.1.117] |
| 1706 | HTH-type transcriptional repressor purR (Purine nucleotide synthesisrepressor) (purR) |
| 1707 | phosphoenolpyruvate carboxylase (ppc) [4.1.1.31] |
| 1708 | YcjX |
| 1709 | Peptide transport periplasmic protein sapA precursor (sapA) |
| 1710 | Peptide transport system permease protein sapB (sapB) |
| 1711 | Peptide transport system permease protein sapC (sapC) |
| 1712 | Peptide transport system ATP-binding protein sapD (sapD) |
| 1713 | Peptide transport system ATP-binding protein sapF (sapF) |
| 1714 | membrane protein, putative |
| 1715 | tRNA pseudouridine synthase A (truA) [4.2.1.70] |
| 1716 | fructose-1,6-bisphosphatase (ftp) [3.1.3.11] |
| 1717 | pyridoxine biosynthesis protein |
| 1718 | 2-deoxy-scyllo-inosose synthase 20 kDa subunit |
| 1719 | D-lactate dehydrogenase (dld) [1.1.1.28] |
| 1720 | Type I site-specific deoxyribonuclease HsdR [3.1.21.3] |
| 1721 | aerobic respiration control sensor protein [2.7.3.—] |
| 1722 | Lipoprotein spr precursor (spr) |
| 1723 | TldD (CSRA) |
| 1724 | conserved hypothetical protein TIGR00096 |
| 1725 | LppC |
| 1726 | conserved hypothetical protein TIGR00252 |
| 1727 | conserved possible phosphoheptose isomerase (gmhA) [5.—.—.—] |
| 1728 | 21 kDa hemolysin precursor |
| 1729 | ribonucleoside-diphosphate reductase alpha chain (nrdA) [1.17.4.1] |
| 1730 | ribonucleoside-diphosphate reductase, beta subunit [1.17.4.1] |
| 1731 | 2-oxoglutarate dehydrogenase, E2 component, dihydrolipoamide succinyltransferase (sucB) [2.3.1.61] |
| 1732 | 2-oxoglutarate dehydrogenase, E1 component (sucA) [1.2.4.2] |
| 1733 | metallo-beta-lactamase superfamily protein [3.—.—.—] |
| 1734 | 3.1.21.— [3.1.21.—] |
| 1735 | Bacterial protein of unknown function (DUF882) superfamily |
| 1736 | cell wall degradation protein (AE005282) |
| 1737 | Tail-specific protease precursor (Protease Re) (C-terminal-processing peptidase) (prc) [3.4.21.102] |
| 1738 | ProQ |
| 1739 | paraquat-inducible protein A |
| 1740 | Protein (fragment) |
| 1741 | Molybdopterin converting factor subunit 2 (MPT synthase subunit 2)(Molybdopterin synthase subunit 2) |
| | (Molybdenum cofactor biosynthesisprotein E) (Molybdopterin converting factor large subunit) (moaE) |
| 1742 | molybdopterin converting factor, subunit 1 (moaD) |
| 1743 | molybdenum cofactor biosynthesis protein C (moaC) |
| 1744 | Molybdenum cofactor biosynthesis protein A (moaA) |
| 1745 | NorA |
| 1746 | KpsF (kpsF) [5.—.—.—] |
| 1747 | 3-deoxy-D-manno-octulosonate 8-phosphate phosphatase(KDO 8-P phosphatase) [3.1.3.45] |
| 1748 | hypothetical membrane protein, TIGR01666 (yccS) |
| 1749 | Protein HI1681 precursor |
| 1750 | Possible protease sohB (sohB) [3.4.21.—] |
| 1751 | Electron transport complex protein rnfA [1.6.5.—] |
| 1752 | Electron transport complex protein rnfB |
| 1753 | Electron transport complex protein rnfC |
| 1754 | Electron transport complex protein rnfD [1.6.5.—] |
| 1765 | Electron transport complex protein rnfG (rnfG) |
| 1756 | Electron transport complex protein rnfE [1.6.5.—] |
| 1757 | endonuclease III (nth) [4.2.99.18] |
| 1758 | sodium-dependent transporter (SNF family) |
| 1759 | molybdenum ABC transporter, ATP-binding protein (modC) [3.6.3.29] |
| 1760 | molybdate ABC transporter, permease protein (modB) |
| 1761 | molybdenum ABC transporter, periplasmic molybdate-binding protein (modA) |
| 1762 | Transcriptional regulator modE (modE) |
| 1763 | unnamed protein product [2.—.—.—] |
| 1764 | glycosyltransferase [2.—.—.—] |
| 1765 | unnamed protein product [2.—.—.—] |
| 1766 | gljycosyl transferase (putative) [2.—.—.—] |
| 1767 | 2.4.99.— [2.4.99.—] |
| 1768 | Polysaccharide biosynthesis protein domain protein |
| 1769 | Uncharacterized ACR, COG1434 family |
| 1770 | 5-methyltetrahydropteroyltriglutamate--homocysteine S-methyltransferase (metE) [2.1.1.14] |
| 1771 | predicted permease |
| 1772 | predicted permease |
| 1773 | Cytosol aminopeptidase (Leucine aminopeptidase) (LAP)(Leucyl aminopeptidase) (pepA) [3.4.11.1] |
| 1774 | transporter, BCCT family NMB1277 (betT) |
| 1775 | Sensor protein qseC [2.7.3.—] |
| 1776 | Transcriptional regulatory protein qseB |
| 1777 | conserved hypothetical protein TIGR00156 |
| 1778 | Pmi |
| 1779 | Pmi (PMI) [5.3.1.8] |
| 1780 | phosphotransferase system enzyme II, glucose-specific, factor III (crr) [2.7.1.69] |
| 1781 | phosphoenolpyruvate-protein phosphotransferase (ptsI) [2.7.3.9] |
| 1782 | Phosphocarrier protein HPr (Histidine-containing protein) (ptsH) [2.7.1.69] |
| 1783 | 3.6.1.— [3.6.1.—] |
| 1784 | Oligoribonuclease [3.1.—.—] |
| 1785 | undecaprenyl-phosphate alpha-N-acetylglucosaminyltransferase (rfe) [2.7.8.—] |
| 1786 | protein-P-II uridylyltransferase (glnD) [2.7.7.59] |
| 1787 | methionine aminopeptidase, type I (map) [3.4.11.18] |
| 1788 | Protein |
| 1789 | Uncharacterised protein family (UPF0231) superfamily |
| 1790 | penicillin-binding protein 1B (mrcB) |
| 1791 | hypothetical protein |
| 1792 | phosphoribosylaminoimidazole-succinocarboxamide synthase (purC) [6.3.2.6] |
| 1793 | argininosuccinate synthase (argG) [6.3.4.5] |
| 1794 | transporter protein |
| 1795 | Protein (IamB) |
| 1796 | urea amidolyase-related protein |
| 1797 | conserved hypothetical protein TIGR00370 |
| 1798 | hsf |
| 1799 | exoribonuclease II (rnb) [3.1.13.1] |
| 1800 | enoyl-[acyl-carrier-protein] reductase (NADH2) (fabI) [1.3.1.9] |
| 1801 | peptide chain release factor 3 (prfC) |
| 1802 | conserved hypothetical protein |
| 1803 | Branched-chain amino acid transport protein azlD (braE) |
| 1804 | branched-chain amino acid transport protein AzlC (azlC) |
| 1805 | HTH-type transcriptional regulator metR (metR) |
| 1806 | L-lactate dehydrogenase (Cytochrome) (lctD) [1.1.2.3] |
| 1807 | glutamate racemase (murI) [5.1.1.3] |
| 1808 | ATP-dependent DNA helicase RecG (recG) [3.6.1.—] |
| 1809 | Guanosine-3′,5′-bis(Diphosphate) 3′-pyrophosphohydrolase((ppGpp)ase) (Penta-phosphateguanosine-3′- |
| | pyrophosphohydrolase) (spoT) [3.1.7.2] |
| 1810 | DNA-directed RNA polymerase omega chain (RNAP omegasubunit) (Transcriptase omega chain) (RNA |
| | polymerase omega subunit) (rpoZ) [2.7.7.6] |
| 1811 | Guanylate kinase (GMP kinase) (gmk) [2.7.4.8] |
| 1812 | Glyceraldehyde 3-phosphate dehydrogenase (GAPDH) (gapdH) [1.2.1.12] |
| 1813 | conserved hypothetical protein |
| 1814 | conserved hypothetical protein |
| 1815 | conserved hypothetical protein |
| 1816 | fimbrial protein hifB |
| 1817 | Phage integrase family domain protein |
| 1818 | Phage integrase family domain protein |
| 1819 | conserved hypothetical protein |
| 1820 | AcrB/AcrD/AcrF family protein (AP001520) |
| 1821 | quinone oxidoreductase (Human) [1.1.1.—] |
| 1822 | arsenical-resistance protein acr3 |
| 1823 | regulatory protein (merR2) |
| 1824 | cation efflux family protein superfamily |
| 1825 | similar to possible arsenic resistance membrane protein ArsB (ArsB) |
| 1826 | arsenate reductase (arsC) [1.20.4.1] |
| 1827 | ArsR-like protein (AF173880) |
| 1828 | RC180 |
| 1829 | ParB-related protein |
| 1830 | conserved hypothetical protein |
| 1831 | predicted protein |
| 1832 | conserved hypothetical protein |
| 1833 | Minor fimbrial subunit hifE precursor |
| 1834 | Minor fimbrial subunit hifD precursor (pilA) |
| 1835 | Outer membrane usher protein hifC precursor |
| 1836 | hypothetical protein |
| 1837 | araC-type sugar metabolism regulator |
| 1838 | gp15 |
| 1839 | hypothetical protein |
| 1840 | hypothetical protein |
| 1841 | KIAA0853 protein, putative |
| 1842 | prophage pi1 protein 11, recombinase (P33) |
| 1843 | hypothetical protein |
| 1844 | single stranded DNA-binding protein (SSB) |
| 1845 | transcriptional regulator, Cro/CI family |
| 1846 | hypothetical protein |
| 1847 | recombination endonuclease |
| 1848 | elongation factor Tu (EF-Tu) |
| 1849 | gene 50 protein |
| 1850 | P protein, putative |
| 1851 | Sb42 |
| 1852 | Roi |
| 1853 | phage regulatory protein YPO2100 |

### REFERENCES

### The Contents of which are Hereby Incorporated by Reference

[1] Fleischmann et al. (1995) Science 269:496-512.
[2] GenBank accession NC_{—}000907.
[3] Geysen et al. (1984) PNAS USA 81:3998-4002.
[4] Carter (1994) Methods Mol Biol. 36:207-23.
[5] Jameson, B A et al. 1988, CABIOS 4(1):181-186.
[6] Raddrizzani & Hammer (2000) Brief Bioinform 1(2):179-89.
[7] De Lalla et al. (1999) J. Immunol. 163:1725-29.
[8] Brusic et al. (1998) Bioinformatics 14(2):121-30
[9] Meister et al. (1995) Vaccine 13(6):581-91.
[10] Roberts et al. (1996) AIDS Res Hum Retroviruses 12(7):593-610.
[11] Maksyutov & Zagrebelnaya (1993) Comput Appl Biosci 9(3):291-7.
[12] Feller & de la Cruz (1991) Nature 349(6311):720-1.
[13] Hopp (1993) Peptide Research 6:183-190.
[14] Welling et al. (1985) FEBS Lett. 188:215-218.
[15] Davenport et al. (1995) Immunogenetics 42:392-297.
[16] Bodanszky (1993) Principles of Peptide Synthesis (ISBN: 0387564314).
[17] Fields et al. (1997) Meth Enzymol 289: Solid-Phase Peptide Synthesis. ISBN: 0121821900.
[18] Chan & White (2000) Fmoc Solid Phase Peptide Synthesis. ISBN: 0199637245.
[19] Kullmann (1987) Enzymatic Peptide Synthesis. ISBN: 0849368413.
[20] Ibba (1996) Biotechnol Genet Eng Rev 13:197-216.
[21] Breedveld (2000) Lancet 355(9205):735-740.
[22] Gorman & Clark (1990) Semin. Immunol. 2:457-466.
[23] Sambrook et al. (1989) Molecular Cloning: A Laboratory Manual.
[24] Short protocols in molecular biology (4th ed, 1999) Ausubel et al. eds. ISBN 0-471-32938-X.
[25] U.S. Pat. No. 5,707,829
[26] Current Protocols in Molecular Biology (F. M. Ausubel et al. eds., 1987) Supplement 30.
[27] EP-B-0509612.
[28] EP-B-0505012.
[29] Yadav et al. (2003) Lett Appl Microbiol 37(3):190-5.
[30] Singhi et al. (2002) Ann Trop Paediatr 22(4):347-53.
[31] Tang et al. (1997) Clin. Chem. 43:2021-2038.
[32] Vaccine Design . . . (1995) eds. Powell & Newman. ISBN: 030644867X. Plenum.
[33] WO00/23105.
[34] WO90/14837.
[35] U.S. Pat. No. 5,057,540.
[36] WO96/33739.
[37] EP-A-0109942.
[38] WO96/11711.
[39] WO00/07621.
[40] Barr et al. (1998) Advanced Drug Delivery Reviews 32:247-271.
[41] Sjolanderet et al. (1998) Advanced Drug Delivery Reviews 32:321-338.
[42] Niikura et al. (2002) Virology 293:273-280.
[43] Lenz et al. (2001) J Immunol 166:5346-5355.
[44] Pinto et al. (2003) J Infect Dis 188:327-338.
[45] Gerber et al. (2001) Virol 75:4752-4760.
[46] WO03/024480
[47] WO03/024481
[48] Gluck et al. (2002) Vaccine 20:B10-B16.
[49] EP-A-0689454.
[50] Johnson et al. (1999) Bioorg Med Chem Lett 9:2273-2278.
[51] Evans et al. (2003) Expert Rev Vaccines 2:219-229.
[52] Meraldi et al. (2003) Vaccine 21:2485-2491.
[53] Pajak et al. (2003) Vaccine 21:836-842.
[54] Kandimalla et al. (2003) Nucleic Acids Research 31:2393-2400.
[55] WO02/26757.
[56] WO99/62923.
[57] Krieg (2003) Nature Medicine 9:831-835.
[58] McCluskie et al. (2002) FEMS Immunology and Medical Microbiology 32:179-185.
[59] WO98/40100.
[60] U.S. Pat. No. 6,207,646.
[61] U.S. Pat. No. 6,239,116.
[62] U.S. Pat. No. 6,429,199.
[63] Kandimalla et al. (2003) Biochemical Society Transactions 31 (part 3):654-658.
[64] Blackwell et al. (2003) J Immunol 170:4061-4068.
[65] Krieg (2002) Trends Immunol 23:64-65.
[66] WO01/95935.
[67] Kandimalla et al. (2003) BBRC 306:948-953.
[68] Bhagat et al. (2003) BBRC 300:853-861.
[69] WO03/035836.
[70] WO95/17211.
[71] WO98/42375.
[72] Beignon et al. (2002) Infect Immun. 70:3012-3019.
[73] Pizza et al. (2001) Vaccine 19:2534-2541.
[74] Pizza et al. (2000) Int J Med Microbiol 290:455-461.
[75] Scharton-Kersten et al. (2000) Infect Immun 68:5306-5313.
[76] Ryan et al. (1999) Infect Immun 67:6270-6280.
[77] Partidos et al. (1999) Immunol Lett 67:209-216.
[78] Peppoloni et al. (2003) Expert Rev Vaccines 2:285-293.
[79] Pine et al. (2002) J Control Release 85:263-270.
[80] Domenighini et al. (1995) Mol Microbiol 15:1165-1167.
[81] WO99/40936.
[82] WO99/44636.
[83] Singh et all (2001) J Cont Release 70:267-276.
[84] WO99/27960.
[85] U.S. Pat. No. 6,090,406
[86] U.S. Pat. No. 5,916,588
[87] EP-A-0626169.
[88] WO99/52549.
[89] WO01/21207.
[90] WO01/21152.
[91] Andrianov et al. (1998) Biomaterials 19:109-115.
[92] Payne et al. (1998) Adv Drug Delivery Review 31:185-196.
[93] Stanley (2002) Clin Exp Dermatol 27:571-577.
[94] Jones (2003) Curr Opin Investig Drugs 4:214-218.
[95] WO99/11241.
[96] WO94/00153.
[97] WO98/57659.
[98] European patent applications 0835318, 0735898 and 0761231.
[99] WO03/009869.
Almeida & Alpar (1996) J. Drug Targeting 3:455-467.
Agarwal & Mishra (1999) Indian J Exp Biol 37:6-16.
Costantino et al. (1992) Vaccine 10:691-698.
Costantino et al. (1999) Vaccine 17:1251-1263.
International patent application WO03/007985.
Watson (2000) Pediatr Infect Dis J19:331-332.
Rubin (2000) Pediatr Clin North Am 47:269-285, v.
Jedrzejas (2001) Microbiol Mol Biol Rev 65:187-207.
Bell (2000) Pediatr Infect Dis J19:1187-1188.
Iwarson (1995) APMIS 103:321-326.
Gerlich et al. (1990) Vaccine 8 Suppl:S63-68 & 79-80.
Vaccines (1988) eds. Plotkin & Mortimer. ISBN 0-7216-1946-0.
Del Guidice et al. (1998) Molecular Aspects of Medicine 19:1-70.
Gustafsson et al. (1996) N. Engl. J. Med. 334:349-355.
Rappuoli et al. (1991) TIBTECH 9:232-238.
Sutter et al. (2000) Pediatr Clin North Am 47:287-308.
Zimmerman & Spann (1999) Am Fam Physician 59:113-118, 125-126.
McMichael (2000) Vaccine 19 Suppl 1:S101-107.
Schuchat (1999) Lancet 353(9146):51-6.
International patent application WO02/34771.
Dale (1999) Infect Dis Clin North Am 13:227-43, viii.
Ferretti et al. (2001) PNAS USA 98: 4658-4663.
Kuroda et al. (2001) Lancet 357(9264):1225-1240; see also pages 1218-1219.
EP-A-0372501
EP-A-0378881
EP-A-0427347
WO93/17712
WO94/03208
WO98/58668
EP-A-0471177
EP-A-0594610.
WO00/56360
WO91/01146
WO00/61761
WO01/72337
Falugi et al. (2001) Eur J Immunol 31:3816-3824.
Baraldo et al, (2004) Infect Immun. 72:4884-7
WO02/091998.
Kuo et al. (1995) Infect Immun 63:2706-13.
Research Disclosure, 453077 (January 2002)
Needleman & Wunsch (1970) J. Mol. Biol. 48, 443-453.
Rice et al. (2000) Trends Genet 16:276-277.
Gennaro (2000) Remington: The Science and Practice of Pharmacy. 20th edition, ISBN: 0683306472.
Methods In Enzymology (S. Colowick and N. Kaplan, eds., Academic Press, Inc.)
Handbook of Experimental Immunology, Vols. I-IV (D. M. Weir and C. C. Blackwell, eds., 1986, Blackwell Scientific Publications)
Sambrook, et al., Molecular Cloning: A Laboratory Manual (2nd Edition, 1989).
Handbook of Surface and Colloidal Chemistry (Birdi, K. S. ed., CRC Press, 1997)
Short Protocols in Molecular Biology, 4th ed. (Ausubel et al. eds., 1999, John Wiley & Sons)
Molecular Biology Techniques: An Intensive Laboratory Course, (Ream et al., eds., 1998, Academic Press)
PCR (Introduction to Biotechniques Series), 2nd ed. (Newton & Graham eds., 1997, Springer Verlag)
Erdile et al. (1993) Infect Immun 61:81-90.

We claim:

1. A polypeptide comprising an amino acid sequence that has at least 75% sequence identity to one or more of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, 576, 578, 580, 582, 584, 586, 588, 590, 592, 594, 596, 598, 600, 602, 604, 606, 608, 610, 612, 614, 616, 618, 620, 622, 624, 626, 628, 630, 632, 634, 636, 638, 640, 642, 644, 646, 648, 650, 652, 654, 656, 658, 660, 662, 664, 666, 668, 670, 672, 674, 676, 678, 680, 682, 684, 686, 688, 690, 692, 694, 696, 698, 700, 702, 704, 706, 708, 710, 712, 714, 716, 718, 720, 722, 724, 726, 728, 730, 732, 734, 736, 738, 740, 742, 744, 746, 748, 750, 752, 754, 756, 758, 760, 762, 764, 766, 768, 770, 772, 774, 776, 778, 780, 782, 784, 786, 788, 790, 792, 794, 796, 798, 800, 802, 804, 806, 808, 810, 812, 814, 816, 818, 820, 822, 824, 826, 828, 830, 832, 834, 836, 838, 840, 842, 844, 846, 848, 850, 852, 854, 856, 858, 860, 862, 864, 866, 868, 870, 872, 874, 876, 878, 880, 882, 884, 886, 888, 890, 892, 894, 896, 898, 900, 902, 904, 906, 908, 910, 912, 914, 916, 918, 920, 922, 924, 926, 928, 930, 932, 934, 936, 938, 940, 942, 944, 946, 948, 950, 952, 954, 956, 958, 960, 962, 964, 966, 968, 970, 972, 974, 976, 978, 980, 982, 984, 986, 988, 990, 992, 994, 996, 998, 1000, 1002, 1004, 1006, 1008, 1010, 1012, 1014, 1016, 1018, 1020, 1022, 1024, 1026, 1028, 1030, 1032, 1034, 1036, 1038, 1040, 1042, 1044, 1046, 1048, 1050, 1052, 1054, 1056, 1058, 1060, 1062, 1064, 1066, 1068, 1070, 1072, 1074, 1076, 1078, 1080, 1082, 1084, 1086, 1088, 1090, 1092, 1094, 1096, 1098, 1100, 1102, 1104, 1106, 1108, 1110, 1112, 1114, 1116, 1118, 1120, 1122, 1124, 1126, 1128, 1130, 1132, 1134, 1136, 1138, 1140, 1142, 1144, 1146, 1148, 1150, 1152, 1154, 1156, 1158, 1160, 1162, 1164, 1166, 1168, 1170, 1172, 1174, 1176, 1178, 1180, 1182, 1184, 1186, 1188, 1190, 1192, 1194, 1196, 1198, 1200, 1202, 1204, 1206, 1208, 1210, 1212, 1214, 1216, 1218, 1220, 1222, 1224, 1226, 1228, 1230, 1232, 1234, 1236, 1238, 1240, 1242, 1244, 1246, 1248, 1250, 1252, 1254, 1256, 1258, 1260, 1262, 1264, 1266, 1268, 1270, 1272, 1274, 1276, 1278, 1280, 1282, 1284, 1286, 1288, 1290, 1292, 1294, 1296, 1298, 1300, 1302, 1304, 1306, 1308, 1310, 1312, 1314, 1316, 1318, 1320, 1322, 1324, 1326, 1328, 1330, 1332, 1334, 1336, 1338, 1340, 1342, 1344, 1346, 1348, 1350, 1352, 1354, 1356, 1358, 1360, 1362, 1364, 1366, 1368, 1370, 1372, 1374, 1376, 1378, 1380, 1382, 1384, 1386, 1388, 1390, 1392, 1394, 1396, 1398, 1400, 1402, 1404, 1406, 1408, 1410, 1412, 1414, 1416, 1418, 1420, 1422, 1424, 1426, 1428, 1430, 1432, 1434, 1436, 1438, 1440, 1442, 1444, 1446, 1448, 1450, 1452, 1454, 1456, 1458, 1460, 1462, 1464, 1466, 1468, 1470, 1472, 1474, 1476, 1478, 1480, 1482, 1484, 1486, 1488, 1490, 1492, 1494, 1496, 1498, 1500, 1502, 1504, 1506, 1508, 1510, 1512, 1514, 1516, 1518, 1520, 1522, 1524, 1526, 1528, 1530, 1532, 1534, 1536, 1538, 1540, 1542, 1544, 1546, 1548, 1550, 1552, 1554, 1556, 1558, 1560, 1562, 1564, 1566, 1568, 1570, 1572, 1574, 1576, 1578, 1580, 1582, 1584, 1586, 1588, 1590, 1592, 1594, 1596, 1598, 1600, 1602, 1604, 1606, 1608, 1610, 1612, 1614, 1616, 1618, 1620, 1622, 1624, 1626, 1628, 1630, 1632, 1634, 1636, 1638, 1640, 1642, 1644, 1646, 1648, 1650, 1652, 1654, 1656, 1658, 1660, 1662, 1664, 1666, 1668, 1670, 1672, 1674, 1676, 1678, 1680, 1682, 1684, 1686, 1688, 1690, 1692, 1694, 1696, 1698, 1700, 1702, 1704, 1706, 1708, 1710, 1712, 1714, 1716, 1718, 1720, 1722, 1724, 1726, 1728, 1730, 1732, 1734, 1736, 1738, 1740, 1742, 1744, 1746, 1748, 1750, 1752, 1754, 1756, 1758, 1760, 1762, 1764, 1766, 1768, 1770, 1772, 1774, 1776, 1778, 1780, 1782, 1784, 1786, 1788, 1790, 1792, 1794, 1796, 1798, 1800, 1802, 1804, 1806, 1808, 1810, 1812, 1814, 1816, 1818, 1820, 1822, 1824, 1826, 1828, 1830, 1832, 1834, 1836, 1838, 1840, 1842, 1844, 1846, 1848, 1850, 1852, 1854, 1856, 1858, 1860, 1862, 1864, 1866, 1868, 1870, 1872, 1874, 1876, 1878, 1880, 1882, 1884, 1886, 1888, 1890, 1892, 1894, 1896, 1898, 1900, 1902, 1904, 1906, 1908, 1910, 1912, 1914, 1916, 1918, 1920, 1922, 1924, 1926, 1928, 1930, 1932, 1934, 1936, 1938, 1940, 1942, 1944, 1946, 1948, 1950, 1952, 1954, 1956, 1958, 1960, 1962, 1964, 1966, 1968, 1970, 1972, 1974, 1976, 1978, 1980, 1982, 1984, 1986, 1988, 1990, 1992, 1994, 1996, 1998, 2000, 2002, 2004, 2006, 2008, 2010, 2012, 2014, 2016, 2018, 2020, 2022, 2024, 2026, 2028, 2030, 2032, 2034, 2036, 2038, 2040, 2042, 2044, 2046, 2048, 2050, 2052, 2054, 2056, 2058, 2060, 2062, 2064, 2066, 2068, 2070, 2072, 2074, 2076, 2078, 2080, 2082, 2084, 2086, 2088, 2090, 2092, 2094, 2096, 2098, 2100, 2102, 2104, 2106, 2108, 2110, 2112, 2114, 2116, 2118, 2120, 2122, 2124, 2126, 2128, 2130, 2132, 2134, 2136, 2138, 2140, 2142, 2144, 2146, 2148, 2150, 2152, 2154, 2156, 2158, 2160, 2162, 2164, 2166, 2168, 2170, 2172, 2174, 2176, 2178, 2180, 2182, 2184, 2186, 2188, 2190, 2192, 2194, 2196, 2198, 2200, 2202, 2204, 2206, 2208, 2210, 2212, 2214, 2216, 2218, 2220, 2222, 2224, 2226, 2228, 2230, 2232, 2234, 2236, 2238, 2240, 2242, 2244, 2246, 2248, 2250, 2252, 2254, 2256, 2258, 2260, 2262, 2264, 2266, 2268, 2270, 2272, 2274, 2276, 2278, 2280, 2282, 2284, 2286, 2288, 2290, 2292, 2294, 2296, 2298, 2300, 2302, 2304, 2306, 2308, 2310, 2312, 2314, 2316, 2318, 2320, 2322, 2324, 2326, 2328, 2330, 2332, 2334, 2336, 2338, 2340, 2342, 2344, 2346, 2348, 2350, 2352, 2354, 2356, 2358, 2360, 2362, 2364, 2366, 2368, 2370, 2372, 2374, 2376, 2378, 2380, 2382, 2384, 2386, 2388, 2390, 2392, 2394, 2396, 2398, 2400, 2462, 2404, 2406, 2408, 2410, 2412, 2414, 2416, 2418, 2420, 2422, 2424, 2426, 2428, 2430, 2432, 2434, 2436, 2438, 2440, 2442, 2444, 2446, 2448, 2450, 2452, 2454, 2456, 2458, 2460, 2462, 2464, 2466, 2468, 2470, 2472, 2474, 2476, 2478, 2480, 2482, 2484, 2486, 2488, 2490, 2492, 2494, 2496, 2498, 2500, 2502, 2504, 2506, 2508, 2510, 2512, 2514, 2516, 2518, 2520, 2522, 2524, 2526, 2528, 2530, 2532, 2534, 2536, 2538, 2540, 2542, 2544, 2546, 2548, 2550, 2552, 2554, 2556, 2558, 2560, 2562, 2564, 2566, 2568, 2570, 2572, 2574, 2576, 2578, 2580, 2582, 2584, 2586, 2588, 2590, 2592, 2594, 2596, 2598, 2600, 2602, 2604, 2606, 2608, 2610, 2612, 2614, 2616, 2618, 2620, 2622, 2624, 2626, 2628, 2630, 2632, 2634, 2636, 2638, 2640, 2642, 2644, 2646, 2648, 2650, 2652, 2654, 2656, 2658, 2660, 2662, 2664, 2666, 2668, 2670, 2672, 2674, 2676, 2678, 2680, 2682, 2684, 2686, 2688, 2690, 2692, 2694, 2696, 2698, 2700, 2702, 2704, 2706, 2708, 2710, 2712, 2714, 2716, 2718, 2720, 2722, 2724, 2726, 2728, 2730, 2732, 2734, 2736, 2738, 2740, 2742, 2744, 2746, 2748, 2750, 2752, 2754, 2756, 2758, 2760, 2762, 2764, 2766, 2768, 2770, 2772, 2774, 2776, 2778, 2780, 2782, 2784, 2786, 2788, 2790, 2792, 2794, 2796, 2798, 2800, 2802, 2804, 2806, 2808, 2810, 2812, 2814, 2816, 2818, 2820, 2822, 2824, 2826, 2828, 2830, 2832, 2834, 2836, 2838, 2840, 2842, 2844, 2846, 2848, 2850, 2852, 2854, 2856, 2858, 2860, 2862, 2864, 2866, 2868, 2870, 2872, 2874, 2876, 2878, 2880, 2882, 2884, 2886, 2888, 2890, 2892, 2894, 2896, 2898, 2900, 2902, 2904, 2906, 2908, 2910, 2912, 2914, 2916, 2918, 2920, 2922, 2924, 2926, 2928, 2930, 2932, 2934, 2936, 2938, 2940, 2942, 2944, 2946, 2948, 2950, 2952, 2954, 2956, 2958, 2960, 2962, 2964, 2966, 2968, 2970, 2972, 2974, 2976, 2978, 2980, 2982, 2984, 2986, 2988, 2990, 2992, 2994, 2996, 2998, 3000, 3002, 3004, 3006, 3008, 3010, 3012, 3014, 3016, 3018, 3020, 3022, 3024, 3026, 3028, 3030, 3032, 3034, 3036, 3038, 3040, 3042, 3044, 3046, 3048, 3050, 3052, 3054, 3056, 3058, 3060, 3062, 3064, 3066, 3068, 3070, 3072, 3074, 3076, 3078, 3080, 3082, 3084, 3086, 3088, 3090, 3092, 3094, 3096, 3098, 3100, 3102, 3104, 3106, 3108, 3110, 3112, 3114, 3116, 3118, 3120, 3122, 3124, 3126, 3128, 3130, 3132, 3134, 3136, 3138, 3140, 3142, 3144, 3146, 3148, 3150, 3152, 3154, 3156, 3158, 3160, 3162, 3164, 3166, 3168, 3170, 3172, 3174, 3176, 3178, 3180, 3182, 3184, 3186, 3188, 3190, 3192, 3194, 3196, 3198, 3200, 3202, 3204, 3206, 3208, 3210, 3212, 3214, 3216, 3218, 3220, 3222, 3224, 3226, 3228, 3230, 3232, 3234, 3236, 3238, 3240, 3242, 3244, 3246, 3248, 3250, 3252, 3254, 3256, 3258, 3260, 3262, 3264, 3266, 3268, 3270, 3272, 3274, 3276, 3278, 3280, 3282, 3284, 3286, 3288, 3290, 3292, 3294, 3296, 3298, 3300, 3302, 3304, 3306, 3308, 3310, 3312, 3314, 3316, 3318, 3320, 3322, 3324, 3326, 3328, 3330, 3332, 3334, 3336, 3338, 3340, 3342, 3344, 3346, 3348, 3350, 3352, 3354, 3356, 3358, 3360, 3362, 3364, 3366, 3368, 3370, 3372, 3374, 3376, 3378, 3380, 3382, 3384, 3386, 3388, 3390, 3392, 3394, 3396, 3398, 3400, 3402, 3404, 3406, 3408, 3410, 3412, 3414, 3416, 3418, 3420, 3422, 3424, 3426, 3428, 3430, 3432, 3434, 3436, 3438, 3440, 3442, 3444, 3446, 3448, 3450, 3452, 3454, 3456, 3458, 3460, 3462, 3464, 3466, 3468, 3470, 3472, 3474, 3476, 3478, 3480, 3482, 3484, 3486, 3488, 3490, 3492, 3494, 3496, 3498, 3500, 3502, 3504, 3506, 3508, 3510, 3512, 3514, 3516, 3518, 3520, 3522, 3524, 3526, 3528, 3530, 3532, 3534, 3536, 3538, 3540, 3542, 3544, 3546, 3548, 3550, 3552, 3554, 3556, 3558, 3560, 3562, 3564, 3566, 3568, 3570, 3572, 3574, 3576, 3578, 3580, 3582, 3584, 3586, 3588, 3590, 3592, 3594, 3596, 3598, 3600, 3602, 3604, 3606, 3608, 3610, 3612, 3614, 3616, 3618, 3620, 3622, 3624, 3626, 3628, 3630, 3632, 3634, 3636, 3638, 3640, 3642, 3644, 3646, 3648, 3650, 3652, 3654, 3656, 3658, 3660, 3662, 3664, 3666, 3668, 3670, 3672, 3674, 3676, 3678, 3680, 3682, 3684, 3686, 3688, 3690, 3692, 3694, 3696, 3698, 3700, 3702, 3704, 3706.

2. An antibody that binds to the polypeptide of claim 1.

3. A monoclonal antibody of claim 2.

4. A nucleic acid comprising an nucleotide sequence that has at least 75% sequence identity to one or more of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 531, 533, 535, 537, 539, 541, 543, 545, 547, 549, 551, 553, 555, 557, 559, 561, 563, 565, 567, 569, 571, 573, 575, 577, 579, 581, 583, 585, 587, 589, 591, 593, 595, 597, 599, 601, 603, 605, 607, 609, 611, 613, 615, 617, 619, 621, 623, 625, 627, 629, 631, 633, 635, 637, 639, 641, 643, 645, 647, 649, 651, 653, 655, 657, 659, 661, 663, 665, 667, 669, 671, 673, 675, 677, 679, 681, 683, 685, 687, 689, 691, 693, 695, 697, 699, 701, 703, 705, 707, 709, 711, 713, 715, 717, 719, 721, 723, 725, 727, 729, 731, 733, 735, 737, 739, 741, 743, 745, 747, 749, 751, 753, 755, 757, 759, 761, 763, 765, 767, 769, 771, 773, 775, 777, 779, 781, 783, 785, 787, 789, 791, 793, 795, 797, 799, 801, 803, 805, 807, 809, 811, 813, 815, 817, 819, 821, 823, 825, 827, 829, 831, 833, 835, 837, 839, 841, 843, 845, 847, 849, 851, 853, 855, 857, 859, 861, 863, 865, 867, 869, 871, 873, 875, 877, 879, 881, 883, 885, 887, 889, 891, 893, 895, 897, 899, 901, 903, 905, 907, 909, 911, 913, 915, 917, 919, 921, 923, 925, 927, 929, 931, 933, 935, 937, 939, 941, 943, 945, 947, 949, 951, 953, 955, 957, 959, 961, 963, 965, 967, 969, 971, 973, 975, 977, 979, 981, 983, 985, 987, 989, 991, 993, 995, 997, 999, 1001, 1003, 1005, 1007, 1009, 1011, 1013, 1015, 1017, 1019, 1021, 1023, 1025, 1027, 1029, 1031, 1033, 1035, 1037, 1039, 1041, 1043, 1045, 1047, 1049, 1051, 1053, 1055, 1057, 1059, 1061, 1063, 1065, 1067, 1069, 1071, 1073, 1075, 1077, 1079, 1081, 1083, 1085, 1087, 1089, 1091, 1093, 1095, 1097, 1099, 1101, 1103, 1105, 1107, 1109, 1111, 1113, 1115, 1117, 1119, 1121, 1123, 1125, 1127, 1129, 1131, 1133, 1135, 1137, 1139, 1141, 1143, 1145, 1147, 1149, 1151, 1153, 1155, 1157, 1159, 1161, 1163, 1165, 1167, 1169, 1171, 1173, 1175, 1177, 1179, 1181, 1183, 1185, 1187, 1189, 1191, 1193, 1195, 1197, 1199, 1201, 1203, 1205, 1207, 1209, 1211, 1213, 1215, 1217, 1219, 1221, 1223, 1225, 1227, 1229, 1231, 1233, 1235, 1237, 1239, 1241, 1243, 1245, 1247, 1249, 1251, 1253, 1255, 1257, 1259, 1261, 1263, 1265, 1267, 1269, 1271, 1273, 1275, 1277, 1279, 1281, 1283, 1285, 1287, 1289, 1291, 1293, 1295, 1297, 1299, 1301, 1303, 1305, 1307, 1309, 1311, 1313, 1315, 1317, 1319, 1321, 1323, 1325, 1327, 1329, 1331, 1333, 1335, 1337, 1339, 1341, 1343, 1345, 1347, 1349, 1351, 1353, 1355, 1357, 1359, 1361, 1363, 1365, 1367, 1369, 1371, 1373, 1375, 1377, 1379, 1381, 1383, 1385, 1387, 1389, 1391, 1393, 1395, 1397, 1399, 1401, 1403, 1405, 1407, 1409, 1411, 1413, 1415, 1417, 1419, 1421, 1423, 1425, 1427, 1429, 1431, 1433, 1435, 1437, 1439, 1441, 1443, 1445, 1447, 1449, 1451, 1453, 1455, 1457, 1459, 1461, 1463, 1465, 1467, 1469, 1471, 1473, 1475, 1477, 1479, 1481, 1483, 1485, 1487, 1489, 1491, 1493, 1495, 1497, 1499, 1501, 1503, 1505, 1507, 1509, 1511, 1513, 1515, 1517, 1519, 1521, 1523, 1525, 1527, 1529, 1531, 1533, 1535, 1537, 1539, 1541, 1543, 1545, 1547, 1549, 1551, 1553, 1555, 1557, 1559, 1561, 1563, 1565, 1567, 1569, 1571, 1573, 1575, 1577, 1579, 1581, 1583, 1585, 1587, 1589, 1591, 1593, 1595, 1597, 1599, 1601, 1603, 1605, 1607, 1609, 1611, 1613, 1615, 1617, 1619, 1621, 1623, 1625, 1627, 1629, 1631, 1633, 1635, 1637, 1639, 1641, 1643, 1645, 1647, 1649, 1651, 1653, 1655, 1657, 1659, 1661, 1663, 1665, 1667, 1669, 1671, 1673, 1675, 1677, 1679, 1681, 1683, 1685, 1687, 1689, 1691, 1693, 1695, 1697, 1699, 1701, 1703, 1705, 1707, 1709, 1711, 1713, 1715, 1717, 1719, 1721, 1723, 1725, 1727, 1729, 1731, 1733, 1735, 1737, 1739, 1741, 1743, 1745, 1747, 1749, 1751, 1753, 1755, 1757, 1759, 1761, 1763, 1765, 1767, 1769, 1771, 1773, 1775, 1777, 1779, 1781, 1783, 1785, 1787, 1789, 1791, 1793, 1795, 1797, 1799, 1801, 1803, 1805, 1807, 1809, 1811, 1813, 1815, 1817, 1819, 1821, 1823, 1825, 1827, 1829, 1831, 1833, 1835, 1837, 1839, 1841, 1843, 1845, 1847, 1849, 1851, 1853, 1855, 1857, 1859, 1861, 1863, 1865, 1867, 1869, 1871, 1873, 1875, 1877, 1879, 1881, 1883, 1885, 1887, 1889, 1891, 1893, 1895, 1897, 1899, 1901, 1903, 1905, 1907, 1909, 1911, 1913, 1915, 1917, 1919, 1921, 1923, 1925, 1927, 1929, 1931, 1933, 1935, 1937, 1939, 1941, 1943, 1945, 1947, 1949, 1951, 1953, 1955, 1957, 1959, 1961, 1963, 1965, 1967, 1969, 1971, 1973, 1975, 1977, 1979, 1981, 1983, 1985, 1987, 1989, 1991, 1993, 1995, 1997, 1999, 2001, 2003, 2005, 2007, 2009, 2011, 2013, 2015, 2017, 2019, 2021, 2023, 2025, 2027, 2029, 2031, 2033, 2035, 2037, 2039, 2041, 2043, 2045, 2047, 2049, 2051, 2053, 2055, 2057, 2059, 2061, 2063, 2065, 2067, 2069, 2071, 2073, 2075, 2077, 2079, 2081, 2083, 2085, 2087, 2089, 2091, 2093, 2095, 2097, 2099, 2101, 2103, 2105, 2107, 2109, 2111, 2113, 2115, 2117, 2119, 2121, 2123, 2125, 2127, 2129, 2131, 2133, 2135, 2137, 2139, 2141, 2143, 2145, 2147, 2149, 2151, 2153, 2155, 2157, 2159, 2161, 2163, 2165, 2167, 2169, 2171, 2173, 2175, 2177, 2179, 2181, 2183, 2185, 2187, 2189, 2191, 2193, 2195, 2197, 2199, 2201, 2203, 2205, 2207, 2209, 2211, 2213, 2215, 2217, 2219, 2221, 2223, 2225, 2227, 2229, 2231, 2233, 2235, 2237, 2239, 2241, 2243, 2245, 2247, 2249, 2251, 2253, 2255, 2257, 2259, 2261, 2263, 2265, 2267, 2269, 2271, 2273, 2275, 2277, 2279, 2281, 2283, 2285, 2287, 2289, 2291, 2293, 2295, 2297, 2299, 2301, 2303, 2305, 2307, 2309, 2311, 2313, 2315, 2317, 2319, 2321, 2323, 2325, 2327, 2329, 2331, 2333, 2335, 2337, 2339, 2341, 2343, 2345, 2347, 2349, 2351, 2353, 2355, 2357, 2359, 2361, 2363, 2365, 2367, 2369, 2371, 2373, 2375, 2377, 2379, 2381, 2383, 2385, 2387, 2389, 2391, 2393, 2395, 2397, 2399, 2401, 2403, 2405, 2407, 2409, 2411, 2413, 2415, 2417, 2419, 2421, 2423, 2425, 2427, 2429, 2431, 2433, 2435, 2437, 2439, 2441, 2443, 2445, 2447, 2449, 2451, 2453, 2455, 2457, 2459, 2461, 2463, 2465, 2467, 2469, 2471, 2473, 2475, 2477, 2479, 2481, 2483, 2485, 2487, 2489, 2491, 2493, 2495, 2497, 2499, 2501, 2503, 2505, 2507, 2509, 2511, 2513, 2515, 2517, 2519, 2521, 2523, 2525, 2527, 2529, 2531, 2533, 2535, 2537, 2539, 2541, 2543, 2545, 2547, 2549, 2551, 2553, 2555, 2557, 2559, 2561, 2563, 2565, 2567, 2569, 2571, 2573, 2575, 2577, 2579, 2581, 2583, 2585, 2587, 2589, 2591, 2593, 2595, 2597, 2599, 2601, 2603, 2605, 2607, 2609, 2611, 2613, 2615, 2617, 2619, 2621, 2623, 2625, 2627, 2629, 2631, 2633, 2635, 2637, 2639, 2641, 2643, 2645, 2647, 2649, 2651, 2653, 2655, 2657, 2659, 2661, 2663, 2665, 2667, 2669, 2671, 2673, 2675, 2677, 2679, 2681, 2683, 2685, 2687, 2689, 2691, 2693, 2695, 2697, 2699, 2701, 2703, 2705, 2707, 2709, 2711, 2713, 2715, 2717, 2719, 2721, 2723, 2725, 2727, 2729, 2731, 2733, 2735, 2737, 2739, 2741, 2743, 2745, 2747, 2749, 2751, 2753, 2755, 2757, 2759, 2761, 2763, 2765, 2767, 2769, 2771, 2773, 2775, 2777, 2779, 2781, 2783, 2785, 2787, 2789, 2791, 2793, 2795, 2797, 2799, 2801, 2803, 2805, 2807, 2809, 2811, 2813, 2815, 2817, 2819, 2821, 2823, 2825, 2827, 2829, 2831, 2833, 2835, 2837, 2839, 2841, 2843, 2845, 2847, 2849, 2851, 2853, 2855, 2857, 2859, 2861, 2863, 2865, 2867, 2869, 2871, 2873, 2875, 2877, 2879, 2881, 2883, 2885, 2887, 2889, 2891, 2893, 2895, 2897, 2899, 2901, 2903, 2905, 2907, 2909, 2911, 2913, 2915, 2917, 2919, 2921, 2923, 2925, 2927, 2929, 2931, 2933, 2935, 2937, 2939, 2941, 2943, 2945, 2947, 2949, 2951, 2953, 2955, 2957, 2959, 2961, 2963, 2965, 2967, 2969, 2971, 2973, 2975, 2977, 2979, 2981, 2983, 2985, 2987, 2989, 2991, 2993, 2995, 2997, 2999, 3001, 3003, 3005, 3007, 3009, 3011, 3013, 3015, 3017, 3019, 3021, 3023, 3025, 3027, 3029, 3031, 3033, 3035, 3037, 3039, 3041, 3043, 3045, 3047, 3049, 3051, 3053, 3055, 3057, 3059, 3061, 3063, 3065, 3067, 3069, 3071, 3073, 3075, 3077, 3079, 3081, 3083, 3085, 3087, 3089, 3091, 3093, 3095, 3097, 3099, 3101, 3103, 3105, 3107, 3109, 3111, 3113, 3115, 3117, 3119, 3121, 3123, 3125, 3127, 3129, 3131, 3133, 3135, 3137, 3139, 3141, 3143, 3145, 3147, 3149, 3151, 3153, 3155, 3157, 3159, 3161, 3163, 3165, 3167, 3169, 3171, 3173, 3175, 3177, 3179, 3181, 3183, 3185, 3187, 3189, 3191, 3193, 3195, 3197, 3199, 3201, 3203, 3205, 3207, 3209, 3211, 3213, 3215, 3217, 3219, 3221, 3223, 3225, 3227, 3229, 3231, 3233, 3235, 3237, 3239, 3241, 3243, 3245, 3247, 3249, 3251, 3253, 3255, 3257, 3259, 3261, 3263, 3265, 3267, 3269, 3271, 3273, 3275, 3277, 3279, 3281, 3283, 3285, 3287, 3289, 3291, 3293, 3295, 3297, 3299, 3301, 3303, 3305, 3307, 3309, 3311, 3313, 3315, 3317, 3319, 3321, 3323, 3325, 3327, 3329, 3331, 3333, 3335, 3337, 3339, 3341, 3343, 3345, 3347, 3349, 3351, 3353, 3355, 3357, 3359, 3361, 3363, 3365, 3367, 3369, 3371, 3373, 3375, 3377, 3379, 3381, 3383, 3385, 3387, 3389, 3391, 3393, 3395, 3397, 3399, 3401, 3403, 3405, 3407, 3409, 3411, 3413, 3415, 3417, 3419, 3421, 3423, 3425, 3427, 3429, 3431, 3433, 3435, 3437, 3439, 3441, 3443, 3445, 3447, 3449, 3451, 3453, 3455, 3457, 3459, 3461, 3463, 3465, 3467, 3469, 3471, 3473, 3475, 3477, 3479, 3481, 3483, 3485, 3487, 3489, 3491, 3493, 3495, 3497, 3499, 3501, 3503, 3505, 3507, 3509, 3511, 3513, 3515, 3517, 3519, 3521, 3523, 3525, 3527, 3529, 3531, 3533, 3535, 3537, 3539, 3541, 3543, 3545, 3547, 3549, 3551, 3553, 3555, 3557, 3559, 3561, 3563, 3565, 3567, 3569, 3571, 3573, 3575, 3577, 3579, 3581, 3583, 3585, 3587, 3589, 3591, 3593, 3595, 3597, 3599, 3601, 3603, 3605, 3607, 3609, 3611, 3613, 3615, 3617, 3619, 3621, 3623, 3625, 3627, 3629, 3631, 3633, 3635, 3637, 3639, 3641, 3643, 3645, 3647, 3649, 3651, 3653, 3655, 3657, 3659, 3661, 3663, 3665, 3667, 3669, 3671, 3673, 3675, 3677, 3679, 3681, 3683, 3685, 3687, 3689, 3691, 3693, 3695, 3697, 3699, 3701, 3703, 3705.

5. A composition comprising: (a) the polypeptide of claim 1; and (b) a pharmaceutically acceptable carrier.

6. The composition of claim 5, further comprising a vaccine adjuvant.

7. A method of treating a patient, comprising administering to the patient a therapeutically effective amount of the composition of claim 6.

8. The method of claim 7 for preventing bacterial meningitis.
